SEQUENCE LISTING (122 sequences)

SEQ ID NO: 1 - RNA, 29 nt, Artificial
  Aptamer
agguucgaagcuuuugcuuggacgaaccg

SEQ ID NO: 2 - RNA, 23 nt, Artificial
  Aptamer
gaagggacggugcggagaggaga

SEQ ID NO: 3 - RNA, 24 nt, Artificial
  Aptamer
gaaggagaggagaggaagaggaga

SEQ ID NO: 4 - RNA, 22 nt, Artificial
  Aptamer
ggaaggauugguaugugguaua

SEQ ID NO: 5 - RNA, 25 nt, Artificial
  Aptamer
cgagggaguggugaggaugaggcga

SEQ ID NO: 6 - RNA, 98 nt, Artificial
  Aptamer
gacgcaacugaaugaaauggugaaggacggguccagguguggcugcuucggcagugcagcuuguugaguagagugugagcuccguaacuagucgcguc

SEQ ID NO: 7 - RNA, 95 nt, Artificial
  Aptamer
gauguaacugaaugaaauggugaaggacggguccaguaggcugcuucggcagccuacuuguugaguagagugugagcuccguaacuaguuacauc

SEQ ID NO: 8 - RNA, 47 nt, Artificial
  Aptamer
agacggucggguccagauauucguaucugucgaguagagugugggcu

SEQ ID NO: 9 - RNA, 65 nt, Artificial
  Scaffold
  misc_feature: tRNALys
  misc_feature (28)..(31): n is any nucleotide
gcccggauagcucagucgguagagcagnnnncggguccaggguucaagucccuguucgggcgcca

SEQ ID NO: 10 - RNA, 86 nt, Artificial
  Scaffold
  misc_feature (23)..(26): n is any nucleotide
ugccuggcgaccauagcgauugnnnncaauuagcgccgaugguagugugggguuuccccaugugagaguaggacaucgccaggcau

SEQ ID NO: 11 - RNA, 62 nt, Artificial
  Scaffold
  misc_feature: F29
  misc_feature (19)..(22): n is any nucleotide
  misc_feature (43)..(46): n is any nucleotide
uugucacguguaugugggnnnncccacauacuuuguugauccnnnnggaucaaucauggcaa

SEQ ID NO: 12 - RNA, 62 nt, Artificial
  Scaffold
  misc_feature: F30
  misc_feature (19)..(22): n is any nucleotide
  misc_feature (43)..(46): n is any nucleotide
uugccauguguaugugggnnnncccacauacucugaugauccnnnnggaucauucauggcaa

SEQ ID NO: 13 - RNA, 28 nt, Artificial
  Aptamer
cgaggaaggaggucugaggaggucacug

SEQ ID NO: 14 - RNA, 52 nt, Artificial
  Aptamer
agacggucggguccaggugcacaaauguggccuguugaguagcgugugggcu

SEQ ID NO: 15 - RNA, 52 nt, Artificial
  Aptamer
agacggucggguccagucccaacgauguuggcuguugaguagugugugggcu

SEQ ID NO: 16 - RNA, 29 nt, Artificial
  Aptamer
aaggacggguccguugaguagagugugag

SEQ ID NO: 17 - RNA, 90 nt, Artificial
  Scaffold and aptamer
gcccggauagcucagucgguagagcagagguucgaagcuuuugcuuggacgaaccgcggguccaggguucaagucccuguucgggcgcca

SEQ ID NO: 18 - RNA, 84 nt, Artificial
  Scaffold + aptamer
gcccggauagcucagucgguagagcaggaagggacggugcggagaggagacggguccaggguucaagucccuguucgggcgcca

SEQ ID NO: 19 - RNA, 85 nt, Artificial
  RNA scaffold + aptamar
gcccggauagcucagucgguagagcaggaaggagaggagaggaagaggagacggguccaggguucaagucccuguucgggcgcca

SEQ ID NO: 20 - RNA, 83 nt, Artificial
  RNA scaffold + aptamer
gcccggauagcucagucgguagagcagggaaggauugguaugugguauacggguccaggguucaagucccuguucgggcgcca

SEQ ID NO: 21 - RNA, 86 nt, Artificial
  RNA scaffold + aptamer
gcccggauagcucagucgguagagcagcgagggaguggugaggaugaggcgacggguccaggguucaagucccuguucgggcgcca

SEQ ID NO: 22 - RNA, 159 nt, Artificial
  RNA scaffold + aptamer
gcccggauagcucagucgguagagcaggacgcaacugaaugaaauggugaaggacggguccagguguggcugcuucggcagugcagcuuguugaguagagugugagcuccguaacuagucgcguccggguccaggguucaagucccuguucgggcgcca

SEQ ID NO: 23 - RNA, 156 nt, Artificial
  RNA scaffold + aptamer
gcccggauagcucagucgguagagcaggauguaacugaaugaaauggugaaggacggguccaguaggcugcuucggcagccuacuuguugaguagagugugagcuccguaacuaguuacauccggguccaggguucaagucccuguucgggcgcca

SEQ ID NO: 24 - RNA, 108 nt, Artificial
  RNA scaffold + aptamer
gcccggauagcucagucgguagagcagagacggucggguccagauauucguaucugucgaguagagugugggcucggguccaggguucaagucccuguucgggcgcca

SEQ ID NO: 25 - RNA, 89 nt, Artificial
  RNA scaffold + aptamer
gcccggauagcucagucgguagagcagcgaggaaggaggucugaggaggucacugcggguccaggguucaagucccuguucgggcgcca

SEQ ID NO: 26 - RNA, 113 nt, Artificial
  RNA scaffold + aptamer
gcccggauagcucagucgguagagcagagacggucggguccaggugcacaaauguggccuguugaguagcgugugggcucggguccaggguucaagucccuguucgggcgcca

SEQ ID NO: 27 - RNA, 113 nt, Artificial
  RNA scaffold + aptamer
gcccggauagcucagucgguagagcagagacggucggguccagucccaacgauguuggcuguugaguagugugugggcucggguccaggguucaagucccuguucgggcgcca

SEQ ID NO: 28 - RNA, 90 nt, Artificial
  RNA scaffold + aptamer
gcccggauagcucagucgguagagcagaaggacggguccguugaguagagugugagcggguccaggguucaagucccuguucgggcgcca

SEQ ID NO: 29 - RNA, 111 nt, Artificial
  RNA scaffold + aptamer
ugccuggcgaccauagcgauugagguucgaagcuuuugcuuggacgaaccgcaauuagcgccgaugguagugugggguuuccccaugugagaguaggacaucgccaggcau

SEQ ID NO: 30 - RNA, 105 nt, Artificial
  RNA scaffold + aptamer
ugccuggcgaccauagcgauuggaagggacggugcggagaggagacaauuagcgccgaugguagugugggguuuccccaugugagaguaggacaucgccaggcau

SEQ ID NO: 31 - RNA, 106 nt, Artificial
  RNA scaffold + aptamer
ugccuggcgaccauagcgauuggaaggagaggagaggaagaggagacaauuagcgccgaugguagugugggguuuccccaugugagaguaggacaucgccaggcau

SEQ ID NO: 32 - RNA, 104 nt, Artificial
  RNA scaffold + aptamer
ugccuggcgaccauagcgauugggaaggauugguaugugguauacaauuagcgccgaugguagugugggguuuccccaugugagaguaggacaucgccaggcau

SEQ ID NO: 33 - RNA, 107 nt, Artificial
  RNA scaffold + aptamer
ugccuggcgaccauagcgauugcgagggaguggugaggaugaggcgacaauuagcgccgaugguagugugggguuuccccaugugagaguaggacaucgccaggcau

SEQ ID NO: 34 - RNA, 180 nt, Artificial
  RNA scaffold + aptamer
ugccuggcgaccauagcgauuggacgcaacugaaugaaauggugaaggacggguccagguguggcugcuucggcagugcagcuuguugaguagagugugagcuccguaacuagucgcguccaauuagcgccgaugguagugugggguuuccccaugugagaguaggacaucgccaggcau

SEQ ID NO: 35 - RNA, 177 nt, Artificial
  RNA scaffold + aptamer
ugccuggcgaccauagcgauuggauguaacugaaugaaauggugaaggacggguccaguaggcugcuucggcagccuacuuguugaguagagugugagcuccguaacuaguuacauccaauuagcgccgaugguagugugggguuuccccaugugagaguaggacaucgccaggcau

SEQ ID NO: 36 - RNA, 129 nt, Artificial
  RNA scaffold + aptamer
ugccuggcgaccauagcgauugagacggucggguccagauauucguaucugucgaguagagugugggcucaauuagcgccgaugguagugugggguuuccccaugugagaguaggacaucgccaggcau

SEQ ID NO: 37 - RNA, 110 nt, Artificial
  RNA scaffold + aptamer
ugccuggcgaccauagcgauugcgaggaaggaggucugaggaggucacugcaauuagcgccgaugguagugugggguuuccccaugugagaguaggacaucgccaggcau

SEQ ID NO: 38 - RNA, 268 nt, Artificial
  RNA scaffold + aptamer
ugccuggcgaccauagcgauugagacggucggguccaggugcacaaauguggccuguugaguagcgugugggcucaauuagcgccgaugguagugugggguuuccccaugugagaguaggacaucgccaggcauugccuggcgaccauagcgauugagacggucggguccaggugcacaaauguggccuguugaguagcgugugggcucaauuagcgccgaugguagugugggguuuccccaugugagaguaggacaucgccaggcau

SEQ ID NO: 39 - RNA, 134 nt, Artificial
  RNA scaffold + aptamer
ugccuggcgaccauagcgauugagacggucggguccagucccaacgauguuggcuguugaguagugugugggcucaauuagcgccgaugguagugugggguuuccccaugugagaguaggacaucgccaggcau

SEQ ID NO: 40 - RNA, 111 nt, Artificial
  RNA scaffold + aptamer
ugccuggcgaccauagcgauugaaggacggguccguugaguagagugugagcaauuagcgccgaugguagugugggguuuccccaugugagaguaggacaucgccaggcau

SEQ ID NO: 41 - RNA, 87 nt, Artificial
  RNA scaffold + aptamer
  misc_feature (68)..(71): n is any nucleotide
uugucacguguaugugggagguucgaagcuuuugcuuggacgaaccgcccacauacuuuguugauccnnnnggaucaaucauggcaa

SEQ ID NO: 42 - RNA, 81 nt, Artificial
  RNA scaffold + aptamer
  misc_feature (62)..(65): n is any nucleotide
uugucacguguauguggggaagggacggugcggagaggagacccacauacuuuguugauccnnnnggaucaaucauggcaa

SEQ ID NO: 43 - RNA, 82 nt, Artificial
  RNA scaffold + aptamer
  misc_feature (63)..(66): n is any nucleotide
uugucacguguauguggggaaggagaggagaggaagaggagacccacauacuuuguugauccnnnnggaucaaucauggcaa

SEQ ID NO: 44 - RNA, 80 nt, Artificial
  RNA scaffold + aptamer
  misc_feature (61)..(64): n is any nucleotide
uugucacguguaugugggggaaggauugguaugugguauacccacauacuuuguugauccnnnnggaucaaucauggcaa

SEQ ID NO: 45 - RNA, 83 nt, Artificial
  RNA scaffold + aptamer
  misc_feature (64)..(67): n is any nucleotide
uugucacguguaugugggcgagggaguggugaggaugaggcgacccacauacuuuguugauccnnnnggaucaaucauggcaa

SEQ ID NO: 46 - RNA, 156 nt, Artificial
  RNA scaffold + aptamer
  misc_feature (137)..(140): n is any nucleotide
uugucacguguauguggggacgcaacugaaugaaauggugaaggacggguccagguguggcugcuucggcagugcagcuuguugaguagagugugagcuccguaacuagucgcguccccacauacuuuguugauccnnnnggaucaaucauggcaa

SEQ ID NO: 47 - RNA, 153 nt, Artificial
  RNA scaffold + aptamer
  misc_feature (134)..(137): n is any nucleotide
uugucacguguauguggggauguaacugaaugaaauggugaaggacggguccaguaggcugcuucggcagccuacuuguugaguagagugugagcuccguaacuaguuacauccccacauacuuuguugauccnnnnggaucaaucauggcaa

SEQ ID NO: 48 - RNA, 105 nt, Artificial
  RNA scaffold + aptamer
  misc_feature (86)..(89): n is any nucleotide
uugucacguguaugugggagacggucggguccagauauucguaucugucgaguagagugugggcucccacauacuuuguugauccnnnnggaucaaucauggcaa

SEQ ID NO: 49 - RNA, 86 nt, Artificial
  RNA scaffold + aptamer
  misc_feature (67)..(70): n is any nucleotide
uugucacguguaugugggcgaggaaggaggucugaggaggucacugcccacauacuuuguugauccnnnnggaucaaucauggcaa

SEQ ID NO: 50 - RNA, 110 nt, Artificial
  RNA scaffold + aptamer
  misc_feature (91)..(94): n is any nucleotide
uugucacguguaugugggagacggucggguccaggugcacaaauguggccuguugaguagcgugugggcucccacauacuuuguugauccnnnnggaucaaucauggcaa

SEQ ID NO: 51 - RNA, 110 nt, Artificial
  RNA scaffold + aptamer
  misc_feature (91)..(94): n is any nucleotide
uugucacguguaugugggagacggucggguccagucccaacgauguuggcuguugaguagugugugggcucccacauacuuuguugauccnnnnggaucaaucauggcaa

SEQ ID NO: 52 - RNA, 87 nt, Artificial
  RNA scaffold + aptamer
  misc_feature (68)..(71): n is any nucleotide
uugucacguguaugugggaaggacggguccguugaguagagugugagcccacauacuuuguugauccnnnnggaucaaucauggcaa

SEQ ID NO: 53 - RNA, 87 nt, Artificial
  RNA scaffold + aptamer
  misc_feature (68)..(71): n is any nucleotide
uugccauguguaugugggagguucgaagcuuuugcuuggacgaaccgcccacauacucugaugauccnnnnggaucauucauggcaa

SEQ ID NO: 54 - RNA, 81 nt, Artificial
  RNA scaffold + aptamer
  misc_feature (62)..(65): n is any nucleotide
uugccauguguauguggggaagggacggugcggagaggagacccacauacucugaugauccnnnnggaucauucauggcaa

SEQ ID NO: 55 - RNA, 82 nt, Artificial
  RNA scaffold + aptamer
  misc_feature (63)..(66): n is any nucleotide
uugccauguguauguggggaaggagaggagaggaagaggagacccacauacucugaugauccnnnnggaucauucauggcaa

SEQ ID NO: 56 - RNA, 80 nt, Artificial
  RNA scaffold + aptamer
  misc_feature (61)..(64): n is any nucleotide
uugccauguguaugugggggaaggauugguaugugguauacccacauacucugaugauccnnnnggaucauucauggcaa

SEQ ID NO: 57 - RNA, 83 nt, Artificial
  RNA scaffold + aptamer
  misc_feature: F30::Mango4
  misc_feature (64)..(67): n is any nucleotide
uugccauguguaugugggcgagggaguggugaggaugaggcgacccacauacucugaugauccnnnnggaucauucauggcaa

SEQ ID NO: 58 - RNA, 156 nt, Artificial
  RNA scaffold + aptamer
  misc_feature (137)..(140): n is any nucleotide
uugccauguguauguggggacgcaacugaaugaaauggugaaggacggguccagguguggcugcuucggcagugcagcuuguugaguagagugugagcuccguaacuagucgcguccccacauacucugaugauccnnnnggaucauucauggcaa

SEQ ID NO: 59 - RNA, 153 nt, Artificial
  RNA scaffold + aptamer
  misc_feature: F30::Spinach2
  misc_feature (134)..(137): n is any nucleotide
uugccauguguauguggggauguaacugaaugaaauggugaaggacggguccaguaggcugcuucggcagccuacuuguugaguagagugugagcuccguaacuaguuacauccccacauacucugaugauccnnnnggaucauucauggcaa

SEQ ID NO: 60 - RNA, 105 nt, Artificial
  RNA scaffold + aptamer
  misc_feature (86)..(89): n is any nucleotide
uugccauguguaugugggagacggucggguccagauauucguaucugucgaguagagugugggcucccacauacucugaugauccnnnnggaucauucauggcaa

SEQ ID NO: 61 - RNA, 86 nt, Artificial
  RNA scaffold + aptamer
  misc_feature (67)..(70): n is any nucleotide
uugccauguguaugugggcgaggaaggaggucugaggaggucacugcccacauacucugaugauccnnnnggaucauucauggcaa

SEQ ID NO: 62 - RNA, 110 nt, Artificial
  RNA scaffold + aptamer
  misc_feature (91)..(94): n is any nucleotide
uugccauguguaugugggagacggucggguccaggugcacaaauguggccuguugaguagcgugugggcucccacauacucugaugauccnnnnggaucauucauggcaa

SEQ ID NO: 63 - RNA, 110 nt, Artificial
  RNA scaffold + aptamer
  misc_feature (91)..(94): n is any nucleotide
uugccauguguaugugggagacggucggguccagucccaacgauguuggcuguugaguagugugugggcucccacauacucugaugauccnnnnggaucauucauggcaa

SEQ ID NO: 64 - RNA, 87 nt, Artificial
  RNA scaffold + aptamer
  misc_feature (68)..(71): n is any nucleotide
uugccauguguaugugggaaggacggguccguugaguagagugugagcccacauacucugaugauccnnnnggaucauucauggcaa

SEQ ID NO: 65 - RNA, 52 nt, Artificial; identical to SEQ ID NO: 14
  Aptamer

SEQ ID NO: 66 - RNA, 52 nt, Artificial; identical to SEQ ID NO: 15
  Aptamer

SEQ ID NO: 67 - DNA, 61 bp, Artificial
  Pf1 promoter
ctcgagcgggacggtcgaaccagcttcaagcgaccggatgagtatgttacagtagatagcg

SEQ ID NO: 68 - DNA, 105 bp, Artificial
  Target RNA
agcgggagaccgctcgaccttagttctcctgttgcgggggagttcatgggatccaggtacccagggcgaggcttatccattgcactccggatgtgctgacccctg

SEQ ID NO: 69 - DNA, 105 bp, Artificial
  F30 with broccoli in insertion site 1 and TTCG in insertion site
ttgccatgtgtatgtgggagacggtcgggtccagatattcgtatctgtcgagtagagtgtgggctcccacatactctgatgatccttcgggatcattcatggcaa

SEQ ID NO: 70 - DNA, 69 bp, Artificial
  TF1 Terminator sequence
ctagcatagcataaaataacgccccaccttcttaacgggaggtggggcgttatttttacggggatcgct

SEQ ID NO: 71 - DNA, 354 bp, Artificial
  SEQ-ID No 67-70 combined, with XbaI recognition site on 5′-end, SalI recognition site on 3′-end
ctgtctctagactcgagcgggacggtcgaaccagcttcaagcgaccggatgagtatgttacagtagatagcgagcgggagaccgctcgaccttagttctcctgttgcgggggagttcatgggatccaggtacccagggcgaggcttatccattgcactccggatgtgctgacccctgttgccatgtgtatgtgggagacggtcgggtccagatattcgtatctgtcgagtagagtgtgggctcccacatactctgatgatccttcgggatcattcatggcaagctagcatagcataaaataacgccccaccttcttaacgggaggtggggcgttatttttacggtcgacctgtc

SEQ ID NO: 72 - DNA, 249 bp, Artificial
  SEQ-ID No 67, 68 and 70, with XbaI recognition site on 5′-end, SalI recognition site on 3′-end
ctgtctctagactcgagcgggacggtcgaaccagcttcaagcgaccggatgagtatgttacagtagatagcgagcgggagaccgctcgaccttagttctcctgttgcgggggagttcatgggatccaggtacccagggcgaggcttatccattgcactccggatgtgctgacccctggctagcatagcataaaataacgccccaccttcttaacgggaggtggggcgttatttttacggtcgacctgtc

SEQ ID NO: 73 - DNA, 20 bp, Artificial
  pJC1_check_f
tgaagaccgtcaaccaaagg

SEQ ID NO: 74 - DNA, 20 bp, Artificial
  pJC1_check_rev
tgccgggaagctagagtaag

SEQ ID NO: 75 - DNA, 6440 bp, Artificial
  pJC1-PF1-U1A-F30::broccoli/UUCG-TF1
ccgagaagttttttacaaaaggcaaaaactttttcgggatcgacagaaataaaacgatcgacggtacgcaacaaaaaagcgtcaggatcgccgtagagcgattgaagaccgtcaaccaaaggggaagcctccaatcgacgcgacgcgcgctctacggcgatcctgacgcagatttttagctatctgtcgcagcgccctcagggacaagccacccgcacaacgtcgcgagggcgatcagcgacgccgcagggggatcctctagactcgagcgggacggtcgaaccagcttcaagcgaccggatgagtatgttacagtagatagcgagcgggagaccgctcgaccttagttctcctgttgcgggggagttcatgggatccaggtacccagggcgaggcttatccattgcactccggatgtgctgacccctgttgccatgtgtatgtgggagacggtcgggtccagatattcgtatctgtcgagtagagtgtgggctcccacatactctgatgatccttcgggatcattcatggcaagctagcatagcataaaataacgccccaccttcttaacgggaggtggggcgttatttttacggtcgacctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacaccgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcaagtcggtcaagccaagcgcaaccagcggcaccgccgcgagcaacgtcgcaagggcgatcaggggacgatttttgcgaagaatttccacggtaagaatccaatctctcgaatttagggtgaaagaagcttggcataggggtgtgcacgaactcggtggaggaaatttccgcggggcaaggcttcgcgaagcggagtcgcggcagtggctttgaagatctttgggagcagtccttgtgcgcttacgaggtgagccggtggggaaccgttatctgcctatggtgtgagcccccctagagagcttcaagagcaatcagcccgacctagaaaggaggccaagagagagacccctacggggggaaccgttttctgcctacgagatggcacatttactgggaagctttacggcgtcctcgtggaagttcaatgcccgcagacttaagtgctctattcacggtctgacgtgacacgctaaattcagacatagcttcattgattgtcggccacgagccagtctctccctcaacagtcataaaccaacctgcaatggtcaagcgatttcctttagctttcctagcttgtcgttgactggacttagctagtttttctcgctgtgctcgggcgtactcactgtttgggtctttccagcgttctgcggcctttttaccgccacgtcttcccatagtggccagagcttttcgccctcggctgctctgcgtctctgtctgacgagcagggacgactggctggcctttagcgacgtagccgcgcacacgtcgcgccatcgtctggcggtcacgcatcggcggcagatcaggctcacggccgtctgctccgaccgcctgagcgacggtgtaggcacgctcgtaggcgtcgatgatcttggtgtcttttaggcgctcaccagccgcttttaactggtatcccacagtcaaagcgtggcgaaaagccgtctcatcacgggcggcacgccctggagcagtccagaggacacggacgccgtcgatcagctctccagacgcttcagcggcgctcggcaggcttgcttcaagcgtggcaagtgcttttgcttccgcagtggcttttcttgccgcttcgatacgtgcccgtccgctagaaaactcctgctcatagcgttttttaggtttttctgtgcctgagatcatgcgagcaacctccataagatcagctaggcgatccacgcgattgtgctgggcatgccagcggtacgcggtgggatcgtcggagacgtgcagtggccaccggctcagcctatgtgaaaaagcctggtcagcgccgaaaacgcgggtcatttcctcggtcgttgcagccagcaggcgcatattcgggctgctcatgcctgctgcggcatacaccggatcaatgagccagatgagctggcatttcccgctcagtggattcacgccgatccaagctggcgctttttccaggcgtgcccagcgctccaaaatcgcgtagacctcggggtttacgtgctcgattttcccgccggcctggtggctcggcacatcaatgtccaggacaagcacggctgcgtgctgcgcgtgcgtcagagcaacatactggcaccgggcaagcgattttgaaccaactcggtataacttcggctgtgtttctcccgtgtccgggtctttgatccaagcgctggcgaagtcgcgggtcttgctgccctggaaattttctctgcccaggtgagcgaggaattcgcggcggtcttcgctcgtccagccacgtgatcgcagcgcgagctcgggatgggtgtcgaacagatcagcggaaaatttccaggccggtgtgtcaatgtctcgtgaatccgctagagtcatttttgagcgctttctcccaggtttggactgggggttagccgacgccctgtgagttaccgctcacggggcgttcaacatttttcaggtattcgtgcagcttatcgcttcttgccgcctgtgcgctttttcgacgcgcgacgctgctgccgattcggtgcaggtggtggcggcgctgacacgtcctgggcggccacggccacacgaaacgcggcatttacgatgtttgtcatgcctgcgggcaccgcgccacgatcgcggataattctcgctgccgcttccagctctgtgacgaccatggccaaaatttcgctcgggggacgcacttccagcgccatttgcgacctagccgcctccagctcctcggcgtggcgtttgttggcgcgctcgcggctggctgcggcacgacacgcatctgagcaatattttgcgcgccgtcctcgcgggtcaggccggggaggaatcaggccaccgcagtaggcgcaactgattcgatcctccactactgtgcgtcctcctggcgctgccgagcacgcagctcgtcagccagctcctcaagatccgccacgagagtttctaggtcgctcgcggcactggcccagtctcgtgatgctggcgcgtccgtcgtatcgagagctcggaaaaatccgatcaccgtttttaaatcgacggcagcatcgagcgcgtcggactccagcgcgacatcagagagatccatagctgatgattcgggccaattttggtacttcgtcgtgaaggtcatgacaccattataacgaacgttcgttaaagtttttggcggaaaatcacgcggcacgaaaattttcacgaagcgggactttgcgcagctcaggggtgctaaaaattttgtatcgcacttgatttttccgaaagacagattatctgcaaacggtgtgtcgtatttctggcttggtttttaaaaaatctggaatcgaaaatttgcggggcga

SEQ ID NO: 76 - DNA, 6395 bp, Artificial
  pJC1-PF1-U1A-TF1
ccgagaagttttttacaaaaggcaaaaactttttcgggatcgacagaaataaaacgatcgacggtacgcaacaaaaaagcgtcaggatcgccgtagagcgattgaagaccgtcaaccaaaggggaagcctccaatcgacgcgacgcgcgctctacggcgatcctgacgcagatttttagctatctgtcgcagcgccctcagggacaagccacccgcacaacgtcgcgagggcgatcagcgacgccgcagggggatcctctagactcgagcgggacggtcgaaccagcttcaagcgaccggatgagtatgttacagtagatagcgagcgggagaccgctcgaccttagttctcctgttgcgggggagttcatgggatccaggtacccagggcgaggcttatccattgcactccggatgtgctgacccctggctagcatagcataaaataacgccccaccttcttaacgggaggtggggcgttatttttacggggatcgcttcttccgcttcctcgctcactgactcgctgcgctcggtcgttcggctgcgggtcgacctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacaccgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcaagtcggtcaagccaagcgcaaccagcggcaccgccgcgagcaacgtcgcaagggcgatcaggggacgatttttgcgaagaatttccacggtaagaatccaatctctcgaatttagggtgaaagaagcttggcataggggtgtgcacgaactcggtggaggaaatttccgcggggcaaggcttcgcgaagcggagtcgcggcagtggctttgaagatctttgggagcagtccttgtgcgcttacgaggtgagccggtggggaaccgttatctgcctatggtgtgagcccccctagagagcttcaagagcaatcagcccgacctagaaaggaggccaagagagagacccctacggggggaaccgttttctgcctacgagatggcacatttactgggaagctttacggcgtcctcgtggaagttcaatgcccgcagacttaagtgctctattcacggtctgacgtgacacgctaaattcagacatagcttcattgattgtcggccacgagccagtctctccctcaacagtcataaaccaacctgcaatggtcaagcgatttcctttagctttcctagcttgtcgttgactggacttagctagtttttctcgctgtgctcgggcgtactcactgtttgggtctttccagcgttctgcggcctttttaccgccacgtcttcccatagtggccagagcttttcgccctcggctgctctgcgtctctgtctgacgagcagggacgactggctggcctttagcgacgtagccgcgcacacgtcgcgccatcgtctggcggtcacgcatcggcggcagatcaggctcacggccgtctgctccgaccgcctgagcgacggtgtaggcacgctcgtaggcgtcgatgatcttggtgtcttttaggcgctcaccagccgcttttaactggtatcccacagtcaaagcgtggcgaaaagccgtctcatcacgggcggcacgccctggagcagtccagaggacacggacgccgtcgatcagctctccagacgcttcagcggcgctcggcaggcttgcttcaagcgtggcaagtgcttttgcttccgcagtggcttttcttgccgcttcgatacgtgcccgtccgctagaaaactcctgctcatagcgttttttaggtttttctgtgcctgagatcatgcgagcaacctccataagatcagctaggcgatccacgcgattgtgctgggcatgccagcggtacgcggtgggatcgtcggagacgtgcagtggccaccggctcagcctatgtgaaaaagcctggtcagcgccgaaaacgcgggtcatttcctcggtcgttgcagccagcaggcgcatattcgggctgctcatgcctgctgcggcatacaccggatcaatgagccagatgagctggcatttcccgctcagtggattcacgccgatccaagctggcgctttttccaggcgtgcccagcgctccaaaatcgcgtagacctcggggtttacgtgctcgattttcccgccggcctggtggctcggcacatcaatgtccaggacaagcacggctgcgtgctgcgcgtgcgtcagagcaacatactggcaccgggcaagcgattttgaaccaactcggtataacttcggctgtgtttctcccgtgtccgggtctttgatccaagcgctggcgaagtcgcgggtcttgctgccctggaaattttctctgcccaggtgagcgaggaattcgcggcggtcttcgctcgtccagccacgtgatcgcagcgcgagctcgggatgggtgtcgaacagatcagcggaaaatttccaggccggtgtgtcaatgtctcgtgaatccgctagagtcatttttgagcgctttctcccaggtttggactgggggttagccgacgccctgtgagttaccgctcacggggcgttcaacatttttcaggtattcgtgcagcttatcgcttcttgccgcctgtgcgctttttcgacgcgcgacgctgctgccgattcggtgcaggtggtggcggcgctgacacgtcctgggcggccacggccacacgaaacgcggcatttacgatgtttgtcatgcctgcgggcaccgcgccacgatcgcggataattctcgctgccgcttccagctctgtgacgaccatggccaaaatttcgctcgggggacgcacttccagcgccatttgcgacctagccgcctccagctcctcggcgtggcgtttgttggcgcgctcgcggctggctgcggcacgacacgcatctgagcaatattttgcgcgccgtcctcgcgggtcaggccggggaggaatcaggccaccgcagtaggcgcaactgattcgatcctccactactgtgcgtcctcctggcgctgccgagcacgcagctcgtcagccagctcctcaagatccgccacgagagtttctaggtcgctcgcggcactggcccagtctcgtgatgctggcgcgtccgtcgtatcgagagctcggaaaaatccgatcaccgtttttaaatcgacggcagcatcgagcgcgtcggactccagcgcgacatcagagagatccatagctgatgattcgggccaattttggtacttcgtcgtgaaggtcatgacaccattataacgaacgttcgttaaagtttttggcggaaaatcacgcggcacgaaaattttcacgaagcgggactttgcgcagctcaggggtgctaaaaattttgtatcgcacttgatttttccgaaagacagattatctgcaaacggtgtgtcgtatttctggcttggtttttaaaaaatctggaatcgaaaatttgcggggcga

SEQ ID NO: 77 - RNA, 148 nt, Artificial
  Scaffold with two aptamers
uugccauguguaugugggagacggucggguccagauauucguaucugucgaguagagugugggcucccacauacucugaugauccagacggucggguccagauauucguaucugucgaguagagugugggcuggaucauucauggcaa

SEQ ID NO: 78 - RNA, 124 nt, Artificial
  Scaffold with two aptamers
uugccauguguaugugggagacggucggguccagauauucguaucugucgaguagagugugggcucccacauacucugaugauccgaagggacggugcggagaggagaggaucauucauggcaa

SEQ ID NO: 79 - DNA, 1191 bp, Artificial
  SEQ ID NO 81, 82, 83 and 69 combined
cgcacaagcggcggagcatgtggattaattcgatgcaacgcgaagaaccttacctgggcttgacatggaccggatcggcgtagagatacgttttcccttgtggtcggttcacaggtggtgcatggttgtcgtcagctcgtgtcgtgagatgttgggttaagtcccgcaacgagcgcaacccttgtcttatgttgccagcacattgtggtgggtactcatgagagactgccggggttaactcggaggaaggtggggatgacgtcaaatcatcatgccccttatgtccagggcttcacacatgctacaatggtcggtacagcgagttgccacaccgtgaggtggagctaatctcttaaagccggcctcagttcggattggggtctgcaactcgaccccatgaagtcggagtcgctagtaatcgcagatcagcaacgctgcggtgaatacgttcccgggccttgtacacaccgcccgtcacgtcatgaaagttggtaacacccgaagccagtggcccaaccttttaggggggagctgtcgaaggtgggatcggcgattgggacgaagtcgtaacaaggtagccgtaccggaaggtgcggctggatctagattgccatgtgtatgtgggagacggtcgggtccagatattcgtatctgtcgagtagagtgtgggctcccacatactctgatgatccttcgggatcattcatggcaacctcctttctaaggagctttattaacccacatcagactgtgtctggttggtgggttgttggtgttggaacccgtatgtggttgccatcaacatatttttaatcgggtggagatgacccctcgggtgacaacaacacagcaaacagtgctgtgattaataggtggcatgctgttgggtgtctggaatgacatcgcaagcatcaccttttggtggtgtgtgtgggttgtttctaacatcgagcatcgtcaacacgggtagagaatgttgtgttctttggttgtggtgggggtggtgtgttgtgtgagaactgtatagtggacgcgagcatctttatttttttgttttttgttgtgtgataccgaacgcgcccgcactttgtgtgtgggttatagtattttgtttgttgttttgtagggcacacggtggatgccttggcatatcaagccgatgaaggacgtgagaggctgcgttatgcctcg

SEQ ID NO: 80 - DNA, 6867 bp, Artificial
  pk19msb_16s rrna-broccoli
cgcacaagcggcggagcatgtggattaattcgatgcaacgcgaagaaccttacctgggcttgacatggaccggatcggcgtagagatacgttttcccttgtggtcggttcacaggtggtgcatggttgtcgtcagctcgtgtcgtgagatgttgggttaagtcccgcaacgagcgcaacccttgtcttatgttgccagcacattgtggtgggtactcatgagagactgccggggttaactcggaggaaggtggggatgacgtcaaatcatcatgccccttatgtccagggcttcacacatgctacaatggtcggtacagcgagttgccacaccgtgaggtggagctaatctcttaaagccggcctcagttcggattggggtctgcaactcgaccccatgaagtcggagtcgctagtaatcgcagatcagcaacgctgcggtgaatacgttcccgggccttgtacacaccgcccgtcacgtcatgaaagttggtaacacccgaagccagtggcccaaccttttaggggggagctgtcgaaggtgggatcggcgattgggacgaagtcgtaacaaggtagccgtaccggaaggtgcggctggatctagattgccatgtgtatgtgggagacggtcgggtccagatattcgtatctgtcgagtagagtgtgggctcccacatactctgatgatccttcgggatcattcatggcaacctcctttctaaggagctttattaacccacatcagactgtgtctggttggtgggttgttggtgttggaacccgtatgtggttgccatcaacatatttttaatcgggtggagatgacccctcgggtgacaacaacacagcaaacagtgctgtgattaataggtggcatgctgttgggtgtctggaatgacatcgcaagcatcaccttttggtggtgtgtgtgggttgtttctaacatcgagcatcgtcaacacgggtagagaatgttgtgttctttggttgtggtgggggtggtgtgttgtgtgagaactgtatagtggacgcgagcatctttatttttttgttttttgttgtgtgataccgaacgcgcccgcactttgtgtgtgggttatagtattttgtttgttgttttgtagggcacacggtggatgccttggcatatcaagccgatgaaggacgtgagaggctgcgttatgcctcgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctctttggcatcgtctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagcggggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagggcgtcccggaaaacgattccgaagcccaacctttcatagaaggcggcggtggaatcgaaatctcgtgatggcaggttgggcgtcgcttggtcggtcatttcgctcggtacccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcgatcctctagcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcttatcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaatt

SEQ ID NO: 81 - DNA, 601 bp, Artificial
  Target integration site (upstream)
cgcacaagcggcggagcatgtggattaattcgatgcaacgcgaagaaccttacctgggcttgacatggaccggatcggcgtagagatacgttttcccttgtggtcggttcacaggtggtgcatggttgtcgtcagctcgtgtcgtgagatgttgggttaagtcccgcaacgagcgcaacccttgtcttatgttgccagcacattgtggtgggtactcatgagagactgccggggttaactcggaggaaggtggggatgacgtcaaatcatcatgccccttatgtccagggcttcacacatgctacaatggtcggtacagcgagttgccacaccgtgaggtggagctaatctcttaaagccggcctcagttcggattggggtctgcaactcgaccccatgaagtcggagtcgctagtaatcgcagatcagcaacgctgcggtgaatacgttcccgggccttgtacacaccgcccgtcacgtcatgaaagttggtaacacccgaagccagtggcccaaccttttaggggggagctgtcgaaggtgggatcggcgattgggacgaagtcgtaacaaggtagccgtaccggaaggtgcggctgga

SEQ ID NO: 82 - DNA, 1718 bp, Artificial
  5′UTR-luc2 (Photinus pyralis)
ggagacccaagctggctagcgtttaaacttaagcttggcaatccggtactgttggtaaagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 83 - DNA, 479 bp, Artificial
  Target integration site (downstream)
cctcctttctaaggagctttattaacccacatcagactgtgtctggttggtgggttgttggtgttggaacccgtatgtggttgccatcaacatatttttaatcgggtggagatgacccctcgggtgacaacaacacagcaaacagtgctgtgattaataggtggcatgctgttgggtgtctggaatgacatcgcaagcatcaccttttggtggtgtgtgtgggttgtttctaacatcgagcatcgtcaacacgggtagagaatgttgtgttctttggttgtggtgggggtggtgtgttgtgtgagaactgtatagtggacgcgagcatctttatttttttgttttttgttgtgtgataccgaacgcgcccgcactttgtgtgtgggttatagtattttgtttgttgttttgtagggcacacggtggatgccttggcatatcaagccgatgaaggacgtgagaggctgcgttatgcctcg

SEQ ID NO: 84 - DNA, 20 bp, Artificial
  16S rRNA-broccoli_for
gtcttccacgacttctgtgc

SEQ ID NO: 85 - DNA, 20 bp, Artificial
  16S rRNA-broccoli_rev
gtgtaaacctccacaccagc

SEQ ID NO: 86 - DNA, 19 bp, Artificial
  rrnA_rev
cttgacctgggaagttgcg

SEQ ID NO: 87 - DNA, 20 bp, Artificial
  rrnB_rev
gcaagattgcttgctaccac

SEQ ID NO: 88 - DNA, 20 bp, Artificial
  rrnC_rev
agaaactcggagcgaccatc

SEQ ID NO: 89 - DNA, 20 bp, Artificial
  rrnD_rev
cgtcacacatcgctctacag

SEQ ID NO: 90 - DNA, 21 bp, Artificial
  rrnE_rev
gaagccttcccatcaagcatc

SEQ ID NO: 91 - DNA, 20 bp, Artificial
  rrnF_rev
cacatcaaggtgacacggag

SEQ ID NO: 92 - DNA, 25 bp, Artificial
  qPCR_broc_fw
tcatgaaagttggtaacacccgaag

SEQ ID NO: 93 - DNA, 24 bp, Artificial
  qPCR_broc_rev
ttgccatgaatgatcccgaaggat

SEQ ID NO: 94 - DNA, 288 bp, Artificial
  SEQ ID No. 68, 69, 96 and 97 combined, with EcoRI recognition site on 5′-end, HindIII recognition site on 3′-end
gaattctaatacgactcactatagagcgggagaccgctcgaccttagttctcctgttgcgggggagttcatgggatccaggtacccagggcgaggcttatccattgcactccggatgtgctgacccctgttgccatgtgtatgtgggagacggtcgggtccagatattcgtatctgtcgagtagagtgtgggctcccacatactctgatgatccttcgggatcattcatggcaactagcataaccccttggggcctctaaacgggtcttgaggggttttttgaagctt

SEQ ID NO: 95 - DNA, 2917 bp, Artificial
  pUC18-PT7-U1A-F30::broccoli/UUCG-TT7
aattctaatacgactcactatagagcgggagaccgctcgaccttagttctcctgttgcgggggagttcatgggatccaggtacccagggcgaggcttatccattgcactccggatgtgctgacccctgttgccatgtgtatgtgggagacggtcgggtccagatattcgtatctgtcgagtagagtgtgggctcccacatactctgatgatccttcgggatcattcatggcaactagcataaccccttggggcctctaaacgggtcttgaggggttttttgaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacg

SEQ ID NO: 96 - DNA, 18 bp, Artificial
  PT7
taatacgactcactatag

SEQ ID NO: 97 - DNA, 48 bp, Artificial
  TT7
ctagcataaccccttggggcctctaaacgggtcttgaggggttttttg

SEQ ID NO: 98 - DNA, 19 bp, Artificial
  pUC18_check_f
ttccggctcgtatgttgtg

SEQ ID NO: 99 - DNA, 21 bp, Artificial
  pUC18_check_rev
aggcgattaagttgggtaacg

SEQ ID NO: 100 - DNA, 330 bp, Artificial
  SEQ ID No. 67, 68, 101, 70 combined with XbaI recognition site on 5′-end, SalI recognition site on 3′-end
ctgtctctagactcgagcgggacggtcgaaccagcttcaagcgaccggatgagtatgttacagtagatagcgagcgggagaccgctcgaccttagttctcctgttgcgggggagttcatgggatccaggtacccagggcgaggcttatccattgcactccggatgtgctgacccctgggagctcttgccatgtgtatgtgggggaaggattggtatgtggtatacccacatactctgatgatccttcgggatcattcatggcaagctagcatagcataaaataacgccccaccttcttaacgggaggtggggcgttatttttacgtcgac

SEQ ID NO: 101 - DNA, 80 bp, Artificial
  F30 with Mango3 in its insertion site
ttgccatgtgtatgtgggggaaggattggtatgtggtatacccacatactctgatgatccttcgggatcattcatggcaa

SEQ ID NO: 102 - DNA, 6421 bp, Artificial
  pJC1-PF1-U1A-F30::mangoIII-TF1
ctagactcgagcgggacggtcgaaccagcttcaagcgaccggatgagtatgttacagtagatagcgagcgggagaccgctcgaccttagttctcctgttgcgggggagttcatgggatccaggtacccagggcgaggcttatccattgcactccggatgtgctgacccctgggagctcttgccatgtgtatgtgggggaaggattggtatgtggtatacccacatactctgatgatccttcgggatcattcatggcaagctagcatagcataaaataacgccccaccttcttaacgggaggtggggcgttatttttacgtcgacctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacaccgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcaagtcggtcaagccaagcgcaaccagcggcaccgccgcgagcaacgtcgcaagggcgatcaggggacgatttttgcgaagaatttccacggtaagaatccaatctctcgaatttagggtgaaagaagcttggcataggggtgtgcacgaactcggtggaggaaatttccgcggggcaaggcttcgcgaagcggagtcgcggcagtggctttgaagatctttgggagcagtccttgtgcgcttacgaggtgagccggtggggaaccgttatctgcctatggtgtgagcccccctagagagcttcaagagcaatcagcccgacctagaaaggaggccaagagagagacccctacggggggaaccgttttctgcctacgagatggcacatttactgggaagctttacggcgtcctcgtggaagttcaatgcccgcagacttaagtgctctattcacggtctgacgtgacacgctaaattcagacatagcttcattgattgtcggccacgagccagtctctccctcaacagtcataaaccaacctgcaatggtcaagcgatttcctttagctttcctagcttgtcgttgactggacttagctagtttttctcgctgtgctcgggcgtactcactgtttgggtctttccagcgttctgcggcctttttaccgccacgtcttcccatagtggccagagcttttcgccctcggctgctctgcgtctctgtctgacgagcagggacgactggctggcctttagcgacgtagccgcgcacacgtcgcgccatcgtctggcggtcacgcatcggcggcagatcaggctcacggccgtctgctccgaccgcctgagcgacggtgtaggcacgctcgtaggcgtcgatgatcttggtgtcttttaggcgctcaccagccgcttttaactggtatcccacagtcaaagcgtggcgaaaagccgtctcatcacgggcggcacgccctggagcagtccagaggacacggacgccgtcgatcagctctccagacgcttcagcggcgctcggcaggcttgcttcaagcgtggcaagtgcttttgcttccgcagtggcttttcttgccgcttcgatacgtgcccgtccgctagaaaactcctgctcatagcgttttttaggtttttctgtgcctgagatcatgcgagcaacctccataagatcagctaggcgatccacgcgattgtgctgggcatgccagcggtacgcggtgggatcgtcggagacgtgcagtggccaccggctcagcctatgtgaaaaagcctggtcagcgccgaaaacgcgggtcatttcctcggtcgttgcagccagcaggcgcatattcgggctgctcatgcctgctgcggcatacaccggatcaatgagccagatgagctggcatttcccgctcagtggattcacgccgatccaagctggcgctttttccaggcgtgcccagcgctccaaaatcgcgtagacctcggggtttacgtgctcgattttcccgccggcctggtggctcggcacatcaatgtccaggacaagcacggctgcgtgctgcgcgtgcgtcagagcaacatactggcaccgggcaagcgattttgaaccaactcggtataacttcggctgtgtttctcccgtgtccgggtctttgatccaagcgctggcgaagtcgcgggtcttgctgccctggaaattttctctgcccaggtgagcgaggaattcgcggcggtcttcgctcgtccagccacgtgatcgcagcgcgagctcgggatgggtgtcgaacagatcagcggaaaatttccaggccggtgtgtcaatgtctcgtgaatccgctagagtcatttttgagcgctttctcccaggtttggactgggggttagccgacgccctgtgagttaccgctcacggggcgttcaacatttttcaggtattcgtgcagcttatcgcttcttgccgcctgtgcgctttttcgacgcgcgacgctgctgccgattcggtgcaggtggtggcggcgctgacacgtcctgggcggccacggccacacgaaacgcggcatttacgatgtttgtcatgcctgcgggcaccgcgccacgatcgcggataattctcgctgccgcttccagctctgtgacgaccatggccaaaatttcgctcgggggacgcacttccagcgccatttgcgacctagccgcctccagctcctcggcgtggcgtttgttggcgcgctcgcggctggctgcggcacgacacgcatctgagcaatattttgcgcgccgtcctcgcgggtcaggccggggaggaatcaggccaccgcagtaggcgcaactgattcgatcctccactactgtgcgtcctcctggcgctgccgagcacgcagctcgtcagccagctcctcaagatccgccacgagagtttctaggtcgctcgcggcactggcccagtctcgtgatgctggcgcgtccgtcgtatcgagagctcggaaaaatccgatcaccgtttttaaatcgacggcagcatcgagcgcgtcggactccagcgcgacatcagagagatccatagctgatgattcgggccaattttggtacttcgtcgtgaaggtcatgacaccattataacgaacgttcgttaaagtttttggcggaaaatcacgcggcacgaaaattttcacgaagcgggactttgcgcagctcaggggtgctaaaaattttgtatcgcacttgatttttccgaaagacagattatctgcaaacggtgtgtcgtatttctggcttggtttttaaaaaatctggaatcgaaaatttgcggggcgaccgagaagttttttacaaaaggcaaaaactttttcgggatcgacagaaataaaacgatcgacggtacgcaacaaaaaagcgtcaggatcgccgtagagcgattgaagaccgtcaaccaaaggggaagcctccaatcgacgcgacgcgcgctctacggcgatcctgacgcagatttttagctatctgtcgcagcgccctcagggacaagccacccgcacaacgtcgcgagggcgatcagcgacgccgcagggggatcct

SEQ ID NO: 103 - DNA, 336 bp, Artificial
  SEQ ID No. 67, 68, cc2, 70 combined with XbaI recognition site on 5′-end, SalI recognition site on 3′-end
ctgtctctagactcgagcgggacggtcgaaccagcttcaagcgaccggatgagtatgttacagtagatagcgagcgggagaccgctcgaccttagttctcctgttgcgggggagttcatgggatccaggtacccagggcgaggcttatccattgcactccggatgtgctgacccctgggagctcttgccatgtgtatgtgggcgaggaaggaggtctgaggaggtcactgcccacatactctgatgatccttcgggatcattcatggcaagctagcatagcataaaataacgccccaccttcttaacgggaggtggggcgttatttttacgtcgac

SEQ ID NO: 104 - DNA, 86 bp, Artificial
  F30 with corn in its insertion site1 and “TTCG” in insertion site2
ttgccatgtgtatgtgggcgaggaaggaggtctgaggaggtcactgcccacatactctgatgatccttcgggatcattcatggcaa

SEQ ID NO: 105 - DNA, 6427 bp, Artificial
  pJC1-PF1-U1A-F30::corn-TF1
ctagactcgagcgggacggtcgaaccagcttcaagcgaccggatgagtatgttacagtagatagcgagcgggagaccgctcgaccttagttctcctgttgcgggggagttcatgggatccaggtacccagggcgaggcttatccattgcactccggatgtgctgacccctgggagctcttgccatgtgtatgtgggcgaggaaggaggtctgaggaggtcactgcccacatactctgatgatccttcgggatcattcatggcaagctagcatagcataaaataacgccccaccttcttaacgggaggtggggcgttatttttacgtcgacctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacaccgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcaagtcggtcaagccaagcgcaaccagcggcaccgccgcgagcaacgtcgcaagggcgatcaggggacgatttttgcgaagaatttccacggtaagaatccaatctctcgaatttagggtgaaagaagcttggcataggggtgtgcacgaactcggtggaggaaatttccgcggggcaaggcttcgcgaagcggagtcgcggcagtggctttgaagatctttgggagcagtccttgtgcgcttacgaggtgagccggtggggaaccgttatctgcctatggtgtgagcccccctagagagcttcaagagcaatcagcccgacctagaaaggaggccaagagagagacccctacggggggaaccgttttctgcctacgagatggcacatttactgggaagctttacggcgtcctcgtggaagttcaatgcccgcagacttaagtgctctattcacggtctgacgtgacacgctaaattcagacatagcttcattgattgtcggccacgagccagtctctccctcaacagtcataaaccaacctgcaatggtcaagcgatttcctttagctttcctagcttgtcgttgactggacttagctagtttttctcgctgtgctcgggcgtactcactgtttgggtctttccagcgttctgcggcctttttaccgccacgtcttcccatagtggccagagcttttcgccctcggctgctctgcgtctctgtctgacgagcagggacgactggctggcctttagcgacgtagccgcgcacacgtcgcgccatcgtctggcggtcacgcatcggcggcagatcaggctcacggccgtctgctccgaccgcctgagcgacggtgtaggcacgctcgtaggcgtcgatgatcttggtgtcttttaggcgctcaccagccgcttttaactggtatcccacagtcaaagcgtggcgaaaagccgtctcatcacgggcggcacgccctggagcagtccagaggacacggacgccgtcgatcagctctccagacgcttcagcggcgctcggcaggcttgcttcaagcgtggcaagtgcttttgcttccgcagtggcttttcttgccgcttcgatacgtgcccgtccgctagaaaactcctgctcatagcgttttttaggtttttctgtgcctgagatcatgcgagcaacctccataagatcagctaggcgatccacgcgattgtgctgggcatgccagcggtacgcggtgggatcgtcggagacgtgcagtggccaccggctcagcctatgtgaaaaagcctggtcagcgccgaaaacgcgggtcatttcctcggtcgttgcagccagcaggcgcatattcgggctgctcatgcctgctgcggcatacaccggatcaatgagccagatgagctggcatttcccgctcagtggattcacgccgatccaagctggcgctttttccaggcgtgcccagcgctccaaaatcgcgtagacctcggggtttacgtgctcgattttcccgccggcctggtggctcggcacatcaatgtccaggacaagcacggctgcgtgctgcgcgtgcgtcagagcaacatactggcaccgggcaagcgattttgaaccaactcggtataacttcggctgtgtttctcccgtgtccgggtctttgatccaagcgctggcgaagtcgcgggtcttgctgccctggaaattttctctgcccaggtgagcgaggaattcgcggcggtcttcgctcgtccagccacgtgatcgcagcgcgagctcgggatgggtgtcgaacagatcagcggaaaatttccaggccggtgtgtcaatgtctcgtgaatccgctagagtcatttttgagcgctttctcccaggtttggactgggggttagccgacgccctgtgagttaccgctcacggggcgttcaacatttttcaggtattcgtgcagcttatcgcttcttgccgcctgtgcgctttttcgacgcgcgacgctgctgccgattcggtgcaggtggtggcggcgctgacacgtcctgggcggccacggccacacgaaacgcggcatttacgatgtttgtcatgcctgcgggcaccgcgccacgatcgcggataattctcgctgccgcttccagctctgtgacgaccatggccaaaatttcgctcgggggacgcacttccagcgccatttgcgacctagccgcctccagctcctcggcgtggcgtttgttggcgcgctcgcggctggctgcggcacgacacgcatctgagcaatattttgcgcgccgtcctcgcgggtcaggccggggaggaatcaggccaccgcagtaggcgcaactgattcgatcctccactactgtgcgtcctcctggcgctgccgagcacgcagctcgtcagccagctcctcaagatccgccacgagagtttctaggtcgctcgcggcactggcccagtctcgtgatgctggcgcgtccgtcgtatcgagagctcggaaaaatccgatcaccgtttttaaatcgacggcagcatcgagcgcgtcggactccagcgcgacatcagagagatccatagctgatgattcgggccaattttggtacttcgtcgtgaaggtcatgacaccattataacgaacgttcgttaaagtttttggcggaaaatcacgcggcacgaaaattttcacgaagcgggactttgcgcagctcaggggtgctaaaaattttgtatcgcacttgatttttccgaaagacagattatctgcaaacggtgtgtcgtatttctggcttggtttttaaaaaatctggaatcgaaaatttgcggggcgaccgagaagttttttacaaaaggcaaaaactttttcgggatcgacagaaataaaacgatcgacggtacgcaacaaaaaagcgtcaggatcgccgtagagcgattgaagaccgtcaaccaaaggggaagcctccaatcgacgcgacgcgcgctctacggcgatcctgacgcagatttttagctatctgtcgcagcgccctcagggacaagccacccgcacaacgtcgcgagggcgatcagcgacgccgcagggggatcct

SEQ ID NO: 106 - DNA, 1115 bp, Artificial
  dsRNA_PT7-alpha-tubulin-F30::broccoli
taatacgactcactatagggcgaatggagaacatcgcacaggacttcggtaaaaagtgccgattgggcttcgccatctacccggctccgcaggtttccactgccgttgtcgaaccatacaactcggttttgacgacacatgccaccctcgaacacgctgactgcgtattcatgatggataatgaggcgatctatcagatctgtcgtcggaatcttggagttgaacgaccggcgtatcaaaatctcaatcgactgattagccaggccgtttcggcgataaccgcttctctacgtttttccggagcgttgaatgttgacctcaacgaatttcagacgaatctcgtcccctacccgcgaatccatttcccgctcgtcacttatgctccgattatttcggctgagaaggctcatcacgagcaacataacgtactggaatacgtattgccatgtgtatgtgggagacggtcgggtccagatattcgtatctgtcgagtagagtgtgggctcccacatactctgatgatccttcgggatcattcatggcaagtcgacctagcataaccccttggggcctctaaacgggtcttgaggggttttttgctgaaagtatcatccacatatgagtactctaatacgactcactatagggcgattccagtacgttatgttgctcgtgatgagccttctcagccgaaataatcggagcataagtgacgagcgggaaatggattcgcgggtaggggacgagattcgtctgaaattcgttgaggtcaacattcaacgctccggaaaaacgtagagaagcggttatcgccgaaacggcctggctaatcagtcgattgagattttgatacgccggtcgttcaactccaagattccgacgacagatctgatagatcgcctcattatccatcatgaatacgcagtcagcgtgttcgagggtggcatgtgtcgtcaaaaccgagttgtatggttcgacaacggcagtggaaacctgcggagccgggtagatggcgaagcccaatcggcactttttaccgaagtcctgtgcgatgttctccatatagcataaaataacgccccaccttcttaacgggaggtggggcgttattttta

SEQ ID NO: 107 - DNA, 6108 bp, Artificial
  pJC1
ccgagaagttttttacaaaaggcaaaaactttttcgggatcgacagaaataaaacgatcgacggtacgcaacaaaaaagcgtcaggatcgccgtagagcgattgaagaccgtcaaccaaaggggaagcctccaatcgacgcgacgcgcgctctacggcgatcctgacgcagatttttagctatctgtcgcagcgccctcagggacaagccacccgcacaacgtcgcgagggcgatcagcgacgccgcagggggatcctctagagtcgacctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacaccgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcaagtcggtcaagccaagcgcaaccagcggcaccgccgcgagcaacgtcgcaagggcgatcaggggacgatttttgcgaagaatttccacggtaagaatccaatctctcgaatttagggtgaaagaagcttggcataggggtgtgcacgaactcggtggaggaaatttccgcggggcaaggcttcgcgaagcggagtcgcggcagtggctttgaagatctttgggagcagtccttgtgcgcttacgaggtgagccggtggggaaccgttatctgcctatggtgtgagcccccctagagagcttcaagagcaatcagcccgacctagaaaggaggccaagagagagacccctacggggggaaccgttttctgcctacgagatggcacatttactgggaagctttacggcgtcctcgtggaagttcaatgcccgcagacttaagtgctctattcacggtctgacgtgacacgctaaattcagacatagcttcattgattgtcggccacgagccagtctctccctcaacagtcataaaccaacctgcaatggtcaagcgatttcctttagctttcctagcttgtcgttgactggacttagctagtttttctcgctgtgctcgggcgtactcactgtttgggtctttccagcgttctgcggcctttttaccgccacgtcttcccatagtggccagagcttttcgccctcggctgctctgcgtctctgtctgacgagcagggacgactggctggcctttagcgacgtagccgcgcacacgtcgcgccatcgtctggcggtcacgcatcggcggcagatcaggctcacggccgtctgctccgaccgcctgagcgacggtgtaggcacgctcgtaggcgtcgatgatcttggtgtcttttaggcgctcaccagccgcttttaactggtatcccacagtcaaagcgtggcgaaaagccgtctcatcacgggcggcacgccctggagcagtccagaggacacggacgccgtcgatcagctctccagacgcttcagcggcgctcggcaggcttgcttcaagcgtggcaagtgcttttgcttccgcagtggcttttcttgccgcttcgatacgtgcccgtccgctagaaaactcctgctcatagcgttttttaggtttttctgtgcctgagatcatgcgagcaacctccataagatcagctaggcgatccacgcgattgtgctgggcatgccagcggtacgcggtgggatcgtcggagacgtgcagtggccaccggctcagcctatgtgaaaaagcctggtcagcgccgaaaacgcgggtcatttcctcggtcgttgcagccagcaggcgcatattcgggctgctcatgcctgctgcggcatacaccggatcaatgagccagatgagctggcatttcccgctcagtggattcacgccgatccaagctggcgctttttccaggcgtgcccagcgctccaaaatcgcgtagacctcggggtttacgtgctcgattttcccgccggcctggtggctcggcacatcaatgtccaggacaagcacggctgcgtgctgcgcgtgcgtcagagcaacatactggcaccgggcaagcgattttgaaccaactcggtataacttcggctgtgtttctcccgtgtccgggtctttgatccaagcgctggcgaagtcgcgggtcttgctgccctggaaattttctctgcccaggtgagcgaggaattcgcggcggtcttcgctcgtccagccacgtgatcgcagcgcgagctcgggatgggtgtcgaacagatcagcggaaaatttccaggccggtgtgtcaatgtctcgtgaatccgctagagtcatttttgagcgctttctcccaggtttggactgggggttagccgacgccctgtgagttaccgctcacggggcgttcaacatttttcaggtattcgtgcagcttatcgcttcttgccgcctgtgcgctttttcgacgcgcgacgctgctgccgattcggtgcaggtggtggcggcgctgacacgtcctgggcggccacggccacacgaaacgcggcatttacgatgtttgtcatgcctgcgggcaccgcgccacgatcgcggataattctcgctgccgcttccagctctgtgacgaccatggccaaaatttcgctcgggggacgcacttccagcgccatttgcgacctagccgcctccagctcctcggcgtggcgtttgttggcgcgctcgcggctggctgcggcacgacacgcatctgagcaatattttgcgcgccgtcctcgcgggtcaggccggggaggaatcaggccaccgcagtaggcgcaactgattcgatcctccactactgtgcgtcctcctggcgctgccgagcacgcagctcgtcagccagctcctcaagatccgccacgagagtttctaggtcgctcgcggcactggcccagtctcgtgatgctggcgcgtccgtcgtatcgagagctcggaaaaatccgatcaccgtttttaaatcgacggcagcatcgagcgcgtcggactccagcgcgacatcagagagatccatagctgatgattcgggccaattttggtacttcgtcgtgaaggtcatgacaccattataacgaacgttcgttaaagtttttggcggaaaatcacgcggcacgaaaattttcacgaagcgggactttgcgcagctcaggggtgctaaaaattttgtatcgcacttgatttttccgaaagacagattatctgcaaacggtgtgtcgtatttctggcttggtttttaaaaaatctggaatcgaaaatttgcggggcga

SEQ ID NO: 108 - DNA, 7236 bp, Artificial
  pJC1_dsRNA_PT7-alpha-tubulin-F30::broccoli
aacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacaccgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcaagtcggtcaagccaagcgcaaccagcggcaccgccgcgagcaacgtcgcaagggcgatcaggggacgatttttgcgaagaatttccacggtaagaatccaatctctcgaatttagggtgaaagaagcttggcataggggtgtgcacgaactcggtggaggaaatttccgcggggcaaggcttcgcgaagcggagtcgcggcagtggctttgaagatctttgggagcagtccttgtgcgcttacgaggtgagccggtggggaaccgttatctgcctatggtgtgagcccccctagagagcttcaagagcaatcagcccgacctagaaaggaggccaagagagagacccctacggggggaaccgttttctgcctacgagatggcacatttactgggaagctttacggcgtcctcgtggaagttcaatgcccgcagacttaagtgctctattcacggtctgacgtgacacgctaaattcagacatagcttcattgattgtcggccacgagccagtctctccctcaacagtcataaaccaacctgcaatggtcaagcgatttcctttagctttcctagcttgtcgttgactggacttagctagtttttctcgctgtgctcgggcgtactcactgtttgggtctttccagcgttctgcggcctttttaccgccacgtcttcccatagtggccagagcttttcgccctcggctgctctgcgtctctgtctgacgagcagggacgactggctggcctttagcgacgtagccgcgcacacgtcgcgccatcgtctggcggtcacgcatcggcggcagatcaggctcacggccgtctgctccgaccgcctgagcgacggtgtaggcacgctcgtaggcgtcgatgatcttggtgtcttttaggcgctcaccagccgcttttaactggtatcccacagtcaaagcgtggcgaaaagccgtctcatcacgggcggcacgccctggagcagtccagaggacacggacgccgtcgatcagctctccagacgcttcagcggcgctcggcaggcttgcttcaagcgtggcaagtgcttttgcttccgcagtggcttttcttgccgcttcgatacgtgcccgtccgctagaaaactcctgctcatagcgttttttaggtttttctgtgcctgagatcatgcgagcaacctccataagatcagctaggcgatccacgcgattgtgctgggcatgccagcggtacgcggtgggatcgtcggagacgtgcagtggccaccggctcagcctatgtgaaaaagcctggtcagcgccgaaaacgcgggtcatttcctcggtcgttgcagccagcaggcgcatattcgggctgctcatgcctgctgcggcatacaccggatcaatgagccagatgagctggcatttcccgctcagtggattcacgccgatccaagctggcgctttttccaggcgtgcccagcgctccaaaatcgcgtagacctcggggtttacgtgctcgattttcccgccggcctggtggctcggcacatcaatgtccaggacaagcacggctgcgtgctgcgcgtgcgtcagagcaacatactggcaccgggcaagcgattttgaaccaactcggtataacttcggctgtgtttctcccgtgtccgggtctttgatccaagcgctggcgaagtcgcgggtcttgctgccctggaaattttctctgcccaggtgagcgaggaattcgcggcggtcttcgctcgtccagccacgtgatcgcagcgcgagctcgggatgggtgtcgaacagatcagcggaaaatttccaggccggtgtgtcaatgtctcgtgaatccgctagagtcatttttgagcgctttctcccaggtttggactgggggttagccgacgccctgtgagttaccgctcacggggcgttcaacatttttcaggtattcgtgcagcttatcgcttcttgccgcctgtgcgctttttcgacgcgcgacgctgctgccgattcggtgcaggtggtggcggcgctgacacgtcctgggcggccacggccacacgaaacgcggcatttacgatgtttgtcatgcctgcgggcaccgcgccacgatcgcggataattctcgctgccgcttccagctctgtgacgaccatggccaaaatttcgctcgggggacgcacttccagcgccatttgcgacctagccgcctccagctcctcggcgtggcgtttgttggcgcgctcgcggctggctgcggcacgacacgcatctgagcaatattttgcgcgccgtcctcgcgggtcaggccggggaggaatcaggccaccgcagtaggcgcaactgattcgatcctccactactgtgcgtcctcctggcgctgccgagcacgcagctcgtcagccagctcctcaagatccgccacgagagtttctaggtcgctcgcggcactggcccagtctcgtgatgctggcgcgtccgtcgtatcgagagctcggaaaaatccgatcaccgtttttaaatcgacggcagcatcgagcgcgtcggactccagcgcgacatcagagagatccatagctgatgattcgggccaattttggtacttcgtcgtgaaggtcatgacaccattataacgaacgttcgttaaagtttttggcggaaaatcacgcggcacgaaaattttcacgaagcgggactttgcgcagctcaggggtgctaaaaattttgtatcgcacttgatttttccgaaagacagattatctgcaaacggtgtgtcgtatttctggcttggtttttaaaaaatctggaatcgaaaatttgcggggcgaccgagaagttttttacaaaaggcaaaaactttttcgggatcgacagaaataaaacgatcgacggtacgcaacaaaaaagcgtcaggatcgccgtagagcgattgaagaccgtcaaccaaaggggaagcctccaatcgacgcgacgcgcgctctacggcgatcctgacgcagatttttagctatctgtcgcagcgccctcagggacaagccacccgcacaacgtcgcgagggcgatcagcgacgccgcagggggatccggatcctctagactaatacgactcactatagggcgaatggagaacatcgcacaggacttcggtaaaaagtgccgattgggcttcgccatctacccggctccgcaggtttccactgccgttgtcgaaccatacaactcggttttgacgacacatgccaccctcgaacacgctgactgcgtattcatgatggataatgaggcgatctatcagatctgtcgtcggaatcttggagttgaacgaccggcgtatcaaaatctcaatcgactgattagccaggccgtttcggcgataaccgcttctctacgtttttccggagcgttgaatgttgacctcaacgaatttcagacgaatctcgtcccctacccgcgaatccatttcccgctcgtcacttatgctccgattatttcggctgagaaggctcatcacgagcaacataacgtactggaatacgtattgccatgtgtatgtgggagacggtcgggtccagatattcgtatctgtcgagtagagtgtgggctcccacatactctgatgatccttcgggatcattcatggcaagtcgacctagcataaccccttggggcctctaaacgggtcttgaggggttttttgctgaaagtatcatccacatatgagtactctaatacgactcactatagggcgattccagtacgttatgttgctcgtgatgagccttctcagccgaaataatcggagcataagtgacgagcgggaaatggattcgcgggtaggggacgagattcgtctgaaattcgttgaggtcaacattcaacgctccggaaaaacgtagagaagcggttatcgccgaaacggcctggctaatcagtcgattgagattttgatacgccggtcgttcaactccaagattccgacgacagatctgatagatcgcctcattatccatcatgaatacgcagtcagcgtgttcgagggtggcatgtgtcgtcaaaaccgagttgtatggttcgacaacggcagtggaaacctgcggagccgggtagatggcgaagcccaatcggcactttttaccgaagtcctgtgcgatgttctccatatagcataaaataacgccccaccttcttaacgggaggtggggcgttatttttaggtaccgatatcgatatccaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacggga

SEQ ID NO: 109 - DNA, 20 bp, Artificial
  egfp_for
atggtgagcaagggcgagga

SEQ ID NO: 110 - DNA, 411 bp, Artificial
  alpha-tubulin RNA from Varroa destructor
atggagaacatcgcacaggacttcggtaaaaagtgccgattgggcttcgccatctacccggctccgcaggtttccactgccgttgtcgaaccatacaactcggttttgacgacacatgccaccctcgaacacgctgactgcgtattcatgatggataatgaggcgatctatcagatctgtcgtcggaatcttggagttgaacgaccggcgtatcaaaatctcaatcgactgattagccaggccgtttcggcgataaccgcttctctacgtttttccggagcgttgaatgttgacctcaacgaatttcagacgaatctcgtcccctacccgcgaatccatttcccgctcgtcacttatgctccgattatttcggctgagaaggctcatcacgagcaacataacgtactggaa

SEQ ID NO: 111 - DNA, 411 bp, Artificial
  alpha-tubulin antisense RNA
ttccagtacgttatgttgctcgtgatgagccttctcagccgaaataatcggagcataagtgacgagcgggaaatggattcgcgggtaggggacgagattcgtctgaaattcgttgaggtcaacattcaacgctccggaaaaacgtagagaagcggttatcgccgaaacggcctggctaatcagtcgattgagattttgatacgccggtcgttcaactccaagattccgacgacagatctgatagatcgcctcattatccatcatgaatacgcagtcagcgtgttcgagggtggcatgtgtcgtcaaaaccgagttgtatggttcgacaacggcagtggaaacctgcggagccgggtagatggcgaagcccaatcggcactttttaccgaagtcctgtgcgatgttctccat

SEQ ID NO: 112 - DNA, 807 bp, Artificial
  5′UTR-egfp (modified from Aequorea victoria)
ggagacccaagctggctagcgtttaaacttaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 113 - DNA, 21 bp, Artificial
  broccoli_rev
ttgccatgaatgatcccgaag

SEQ ID NO: 114 - DNA, 27 bp, Artificial
  luc2_for
atggaagatgccaaaaacattaagaag

SEQ ID NO: 115 - DNA, 11662 bp, Artificial
  pK18mobsacB-DE3
  misc_feature (2314)..(2314): n is a, c, g, or t
ctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaancgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgccccgctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattggctccgttgttcacgtctactacgacggcgacgagaacgacaaggaaaccttcctcatcggtacccgtgctggcgcttccgagaacccagatcttgagacctactctgagcagtccccactcggcgctgcaattctcggagctcaggaaggcgacacccgtcagtacaccgctccaaatggttccgttatctccgtaactgttgtttctgcagaaccatacaactcagcaaaagccgcgacactccgcggcaaaaactaaccaaggatttaaaagtcttcaaaatgacaactctttcacgtaagttcttcgtttctgctaccacagccctggcggcagtcgcactggttgcgtgttcccctaatgagattgattctgaactgaaggtgccaacggcaactggcgtttctttaccttcgaagaacgtttccgcgacctcaactgctactacagatgaggatgcgcctggctacattgattgcgtagccgcaccaactcagcaacctgctgaaatctcactaaactgtgcaatggatattgatcggctcacggatatttcttggagcgaatgggatactgattccgcaactggaaccggtacccgcatcgtaaccgctgcaaatggtcaagagaccgaaaccgaagatattgaggtgaagctttccttccccaccgagtcttcccaaggcctagtgttcactcaggtcaccgtcgatggacaggttctcttcctctaatcctccataattagagagcgtaaggcccctacttcctgttttaggaaataggggccttttgttgtcttctcctggaggctatttaagaagtttaaattgtgtccatgagttcgctcgagaactgcgcaactcgtgaaaggtaggcggatccagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccccaggctttacactttatgcttccggctcgtataatgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgtcgggatctgatccggatttactaactggaagaggcactaaatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaacgaattcgcgtatggcaatgacagtttgagacggccacaggcgattctgagaagccattttctttgggcgccgtggcagtttttattgggtcccaccgccgaactgcatattcgaaccaaggagcctcaaaaatcgagctcgctttggtctcaaacgcacatttatcgcgcgttgaagtgtgcgtttgagaccaaagagccctccacaacgcacgtctttggtttggatatgacaggtgcccaagaactcaccccgccccatgctcacagagcccccatcagaagccaaaagaccccttccctgcccaagaagaacaggatgaaggggtcttgtgctgcgtaaactagcggttttggaagtagctaagcagacgtaggatttcggtgtagagccagaccaaggtcactgcaagaccaagcgcaacgccccatgccatcttggaaggtgcaccttcgcggacgaggcggtcagctgcatcgaagtcggagaggaagctgaatgctgccaggccgatgcagaagagggagaagataatcgcgatgattccaccgtcacgcagtgggcttgcgccaccagtgaacagtgcccatacaacgttgcccaggacaagaaccaggacgccaaccatcatgccggtgaggatgcggttgaacttaggagtgaccttgatagcgccagtcttgtatacaaacagcatgccaatgaatacaccgatggtgccaaggactgcctggccaatgaggccacctgcgttggcgttaccaactgtgaagccggacagcagaagggaaattccgccgacgaagaggccttcgaatactgcgtaaatcaaagtgactgccgcagat

SEQ ID NO: 116 - DNA, 20 bp, Artificial
  DE3_for
tttggcgtgtggttggttag

SEQ ID NO: 117 - DNA, 20 bp, Artificial
  DE3_rev
tctctgagctgctggccaac

SEQ ID NO: 118 - DNA, 990 bp, Artificial
  PT7-egfp-broccoli-TT7 (SEQ ID No. 96, 112, 69 and 97 combined)
taatacgactcactatagggagacccaagctggctagcgtttaaacttaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatacgtattgccatgtgtatgtgggagacggtcgggtccagatattcgtatctgtcgagtagagtgtgggctcccacatactctgatgatccttcgggatcattcatggcaagtcgacctagcataaccccttggggcctctaaacgggtcttgaggggttttttg

SEQ ID NO: 119 - DNA, 1911 bp, Artificial
  PT7-luc2-broccoli-TT7 (SEQ ID No. 96, 82, 69 and 97 combined)
taatacgactcactatagggagacccaagctggctagcgtttaaacttaagcttggcaatccggtactgttggtaaagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaataattctagatacgtattgccatgtgtatgtgggagacggtcgggtccagatattcgtatctgtcgagtagagtgtgggctcccacatactctgatgatccttcgggatcattcatggcaagtcgacctagcataaccccttggggcctctaaacgggtcttgaggggttttttg

SEQ ID NO: 120 - DNA, 7102 bp, Artificial
  pJC1-PT7-egfp-broccoli-TT7
ccgagaagttttttacaaaaggcaaaaactttttcgggatcgacagaaataaaacgatcgacggtacgcaacaaaaaagcgtcaggatcgccgtagagcgattgaagaccgtcaaccaaaggggaagcctccaatcgacgcgacgcgcgctctacggcgatcctgacgcagatttttagctatctgtcgcagcgccctcagggacaagccacccgcacaacgtcgcgagggcgatcagcgacgccgcagggtagtactcagctgtaatacgactcactatagggagacccaagctggctagcgtttaaacttaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatacgtattgccatgtgtatgtgggagacggtcgggtccagatattcgtatctgtcgagtagagtgtgggctcccacatactctgatgatccttcgggatcattcatggcaagtcgacctagcataaccccttggggcctctaaacgggtcttgaggggttttttgctgaaagatatcatccaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacaccgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcaagtcggtcaagccaagcgcaaccagcggcaccgccgcgagcaacgtcgcaagggcgatcaggggacgatttttgcgaagaatttccacggtaagaatccaatctctcgaatttagggtgaaagaagcttggcataggggtgtgcacgaactcggtggaggaaatttccgcggggcaaggcttcgcgaagcggagtcgcggcagtggctttgaagatctttgggagcagtccttgtgcgcttacgaggtgagccggtggggaaccgttatctgcctatggtgtgagcccccctagagagcttcaagagcaatcagcccgacctagaaaggaggccaagagagagacccctacggggggaaccgttttctgcctacgagatggcacatttactgggaagctttacggcgtcctcgtggaagttcaatgcccgcagacttaagtgctctattcacggtctgacgtgacacgctaaattcagacatagcttcattgattgtcggccacgagccagtctctccctcaacagtcataaaccaacctgcaatggtcaagcgatttcctttagctttcctagcttgtcgttgactggacttagctagtttttctcgctgtgctcgggcgtactcactgtttgggtctttccagcgttctgcggcctttttaccgccacgtcttcccatagtggccagagcttttcgccctcggctgctctgcgtctctgtctgacgagcagggacgactggctggcctttagcgacgtagccgcgcacacgtcgcgccatcgtctggcggtcacgcatcggcggcagatcaggctcacggccgtctgctccgaccgcctgagcgacggtgtaggcacgctcgtaggcgtcgatgatcttggtgtcttttaggcgctcaccagccgcttttaactggtatcccacagtcaaagcgtggcgaaaagccgtctcatcacgggcggcacgccctggagcagtccagaggacacggacgccgtcgatcagctctccagacgcttcagcggcgctcggcaggcttgcttcaagcgtggcaagtgcttttgcttccgcagtggcttttcttgccgcttcgatacgtgcccgtccgctagaaaactcctgctcatagcgttttttaggtttttctgtgcctgagatcatgcgagcaacctccataagatcagctaggcgatccacgcgattgtgctgggcatgccagcggtacgcggtgggatcgtcggagacgtgcagtggccaccggctcagcctatgtgaaaaagcctggtcagcgccgaaaacgcgggtcatttcctcggtcgttgcagccagcaggcgcatattcgggctgctcatgcctgctgcggcatacaccggatcaatgagccagatgagctggcatttcccgctcagtggattcacgccgatccaagctggcgctttttccaggcgtgcccagcgctccaaaatcgcgtagacctcggggtttacgtgctcgattttcccgccggcctggtggctcggcacatcaatgtccaggacaagcacggctgcgtgctgcgcgtgcgtcagagcaacatactggcaccgggcaagcgattttgaaccaactcggtataacttcggctgtgtttctcccgtgtccgggtctttgatccaagcgctggcgaagtcgcgggtcttgctgccctggaaattttctctgcccaggtgagcgaggaattcgcggcggtcttcgctcgtccagccacgtgatcgcagcgcgagctcgggatgggtgtcgaacagatcagcggaaaatttccaggccggtgtgtcaatgtctcgtgaatccgctagagtcatttttgagcgctttctcccaggtttggactgggggttagccgacgccctgtgagttaccgctcacggggcgttcaacatttttcaggtattcgtgcagcttatcgcttcttgccgcctgtgcgctttttcgacgcgcgacgctgctgccgattcggtgcaggtggtggcggcgctgacacgtcctgggcggccacggccacacgaaacgcggcatttacgatgtttgtcatgcctgcgggcaccgcgccacgatcgcggataattctcgctgccgcttccagctctgtgacgaccatggccaaaatttcgctcgggggacgcacttccagcgccatttgcgacctagccgcctccagctcctcggcgtggcgtttgttggcgcgctcgcggctggctgcggcacgacacgcatctgagcaatattttgcgcgccgtcctcgcgggtcaggccggggaggaatcaggccaccgcagtaggcgcaactgattcgatcctccactactgtgcgtcctcctggcgctgccgagcacgcagctcgtcagccagctcctcaagatccgccacgagagtttctaggtcgctcgcggcactggcccagtctcgtgatgctggcgcgtccgtcgtatcgagagctcggaaaaatccgatcaccgtttttaaatcgacggcagcatcgagcgcgtcggactccagcgcgacatcagagagatccatagctgatgattcgggccaattttggtacttcgtcgtgaaggtcatgacaccattataacgaacgttcgttaaagtttttggcggaaaatcacgcggcacgaaaattttcacgaagcgggactttgcgcagctcaggggtgctaaaaattttgtatcgcacttgatttttccgaaagacagattatctgcaaacggtgtgtcgtatttctggcttggtttttaaaaaatctggaatcgaaaatttgcggggcga

SEQ ID NO: 121 - DNA, 8070 bp, Artificial
  pJC1-PT7-luc2-broccoli-TT7
ggtacgcaacaaaaaagcgtcaggatcgccgtagagcgattgaagaccgtcaaccaaaggggaagcctccaatcgacgcgacgcgcgctctacggcgatcctgacgcagatttttagctatctgtcgcagcgccctcagggacaagccacccgcacaacgtcgcgagggcgatcagcgacgccgcagggggatccagtactcagctggctaactagagaacccactgcttactggcttatcgaaattaatacgactcactatagggagacccaagctggctagcgtttaaacttaagcttggcaatccggtactgttggtaaagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaataattctagatacgtattgccatgtgtatgtgggagacggtcgggtccagatattcgtatctgtcgagtagagtgtgggctcccacatactctgatgatccttcgggatcattcatggcaagtcgacctagcataaccccttggggcctctaaacgggtcttgaggggttttttgctgaaagatatcgatatccaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacaccgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcaagtcggtcaagccaagcgcaaccagcggcaccgccgcgagcaacgtcgcaagggcgatcaggggacgatttttgcgaagaatttccacggtaagaatccaatctctcgaatttagggtgaaagaagcttggcataggggtgtgcacgaactcggtggaggaaatttccgcggggcaaggcttcgcgaagcggagtcgcggcagtggctttgaagatctttgggagcagtccttgtgcgcttacgaggtgagccggtggggaaccgttatctgcctatggtgtgagcccccctagagagcttcaagagcaatcagcccgacctagaaaggaggccaagagagagacccctacggggggaaccgttttctgcctacgagatggcacatttactgggaagctttacggcgtcctcgtggaagttcaatgcccgcagacttaagtgctctattcacggtctgacgtgacacgctaaattcagacatagcttcattgattgtcggccacgagccagtctctccctcaacagtcataaaccaacctgcaatggtcaagcgatttcctttagctttcctagcttgtcgttgactggacttagctagtttttctcgctgtgctcgggcgtactcactgtttgggtctttccagcgttctgcggcctttttaccgccacgtcttcccatagtggccagagcttttcgccctcggctgctctgcgtctctgtctgacgagcagggacgactggctggcctttagcgacgtagccgcgcacacgtcgcgccatcgtctggcggtcacgcatcggcggcagatcaggctcacggccgtctgctccgaccgcctgagcgacggtgtaggcacgctcgtaggcgtcgatgatcttggtgtcttttaggcgctcaccagccgcttttaactggtatcccacagtcaaagcgtggcgaaaagccgtctcatcacgggcggcacgccctggagcagtccagaggacacggacgccgtcgatcagctctccagacgcttcagcggcgctcggcaggcttgcttcaagcgtggcaagtgcttttgcttccgcagtggcttttcttgccgcttcgatacgtgcccgtccgctagaaaactcctgctcatagcgttttttaggtttttctgtgcctgagatcatgcgagcaacctccataagatcagctaggcgatccacgcgattgtgctgggcatgccagcggtacgcggtgggatcgtcggagacgtgcagtggccaccggctcagcctatgtgaaaaagcctggtcagcgccgaaaacgcgggtcatttcctcggtcgttgcagccagcaggcgcatattcgggctgctcatgcctgctgcggcatacaccggatcaatgagccagatgagctggcatttcccgctcagtggattcacgccgatccaagctggcgctttttccaggcgtgcccagcgctccaaaatcgcgtagacctcggggtttacgtgctcgattttcccgccggcctggtggctcggcacatcaatgtccaggacaagcacggctgcgtgctgcgcgtgcgtcagagcaacatactggcaccgggcaagcgattttgaaccaactcggtataacttcggctgtgtttctcccgtgtccgggtctttgatccaagcgctggcgaagtcgcgggtcttgctgccctggaaattttctctgcccaggtgagcgaggaattcgcggcggtcttcgctcgtccagccacgtgatcgcagcgcgagctcgggatgggtgtcgaacagatcagcggaaaatttccaggccggtgtgtcaatgtctcgtgaatccgctagagtcatttttgagcgctttctcccaggtttggactgggggttagccgacgccctgtgagttaccgctcacggggcgttcaacatttttcaggtattcgtgcagcttatcgcttcttgccgcctgtgcgctttttcgacgcgcgacgctgctgccgattcggtgcaggtggtggcggcgctgacacgtcctgggcggccacggccacacgaaacgcggcatttacgatgtttgtcatgcctgcgggcaccgcgccacgatcgcggataattctcgctgccgcttccagctctgtgacgaccatggccaaaatttcgctcgggggacgcacttccagcgccatttgcgacctagccgcctccagctcctcggcgtggcgtttgttggcgcgctcgcggctggctgcggcacgacacgcatctgagcaatattttgcgcgccgtcctcgcgggtcaggccggggaggaatcaggccaccgcagtaggcgcaactgattcgatcctccactactgtgcgtcctcctggcgctgccgagcacgcagctcgtcagccagctcctcaagatccgccacgagagtttctaggtcgctcgcggcactggcccagtctcgtgatgctggcgcgtccgtcgtatcgagagctcggaaaaatccgatcaccgtttttaaatcgacggcagcatcgagcgcgtcggactccagcgcgacatcagagagatccatagctgatgattcgggccaattttggtacttcgtcgtgaaggtcatgacaccattataacgaacgttcgttaaagtttttggcggaaaatcacgcggcacgaaaattttcacgaagcgggactttgcgcagctcaggggtgctaaaaattttgtatcgcacttgatttttccgaaagacagattatctgcaaacggtgtgtcgtatttctggcttggtttttaaaaaatctggaatcgaaaatttgcggggcgaccgagaagttttttacaaaaggcaaaaactttttcgggatcgacagaaataaaacgatcgac

SEQ ID NO: 122 - DNA, 6 bp, Artificial Sequence
  Restriction site for verification of positive integration
tctaga

(19) United States
(12) Patent Application Publication
(10) Pub. No.: US 2024/0294903 A1
(43) Pub. Date: Sep. 5, 2024

(54) RNA-APTAMER-SENSORS

(71) Applicant: SenseUp GmbH, Jülich (DE)

(72) Inventors: Lion Flachbart, Düsseldorf (DE); Regina Mahr, Frechen (DE); Georg Schaumann, Düsseldorf (DE)

(21) Appl. No.: 18/024,028

(22) PCT Filed: Aug. 27, 2021
(86) PCT No.: PCT/EP2021/073813

§ 371 Date: Feb. 28, 2023

(30) Foreign Application Priority Data
Sep. 1, 2020 (EP) 20193971.7

(51) Int. Cl. C12N 15/10; C12N 1/20; C12N 15/115; C12N 15/77; C12R 1/15; G01N 15/149

(52) U.S. Cl. CPC C12N 15/1086; C12N 1/205; C12N 15/115; C12N 15/77; G01N 15/149; C12N 2310/16; C12N 2310/3517; C12N 2501/999; C12R 2001/15

(57) ABSTRACT

The present invention relates to a method for optimizing the production of a RNA sequence of interest in a cell, comprising the steps of a) introducing into a plurality of cells a vector capable of expressing a heterologous RNA of interest, wherein said RNA is tagged with a RNA tag comprising an aptamer capable of stabilizing a fluorophore and a scaffold capable of stabilizing the aptamer; b) culturing the cells in a culture medium under conditions that allow expression of the heterologous RNA of interest; c) adding said fluorophore to the culture medium; and d) identifying those cells that show the highest intensity of fluorescence and therefore a high expression of the RNA of interest.

### CROSS REFERENCE TO RELATED APPLICATIONS

This application is a U.S. National Phase of PCT/EP2021/073813, filed Aug. 27, 2021, which claims priority to European Application No. 20193971.7, filed Sep. 1, 2020, each of which is incorporated herein by reference in their entirety.

### INCORPORATION BY REFERENCE OF MATERIALS SUBMITTED ELECTRONICALLY

This application contains, as a separate part of the disclosure, a Sequence Listing in computer readable form (Filename: 58755_SeqListing.txt; Size: 145,308 bytes; Created: Oct. 11, 2023), which is incorporated by reference in its entirety.

### FIELD OF THE INVENTION

The invention pertains to methods for optimizing the expression of heterologous RNA in cells. Likewise, the invention pertains to cells that allow quantification of the expressed RNA.

### TECHNICAL BACKGROUND

Research in recent years has brought the awareness that RNA, far from being merely a transition molecule, fulfills a variety of functions, both regulatory and enzymatic. For example, it has been described that many small RNAs play key roles in the regulation of gene expression and that higher-order structures in RNA sequences, such as riboswitches or ribozymes, act as regulators of mRNA expression (Breaker, R. R. Natural and engineered nucleic acids as tools to explore biology. Nature 2004:432,838-845. Nellen, W., and C. Hammann. Small RNAs: analysis and regulatory functions. Nucleic acids and molecular biology. Springer-Verlag 2005, Heidelberg, Germany). Based on their function as transition molecules (mRNA), as artificial small interfering RNA-molecules (siRNAs), as catalytically active RNA-molecules (ribozymes), as regulatory or interacting RNA-molecules (RNA-aptamers), RNA has versatile potential as active ingredient in therapeutics, biopesticides and other applications (Khan, A. U. Ribozyme: a clinical tool. Clin. Chim. Acta 2006:367,20-27. Fletcher, S. J., Reeves, P. T., Hoang, B. T. & Mitter, N. A Perspective on RNAi-Based Biopesticides. Front. Plant Sci. 11, (2020). Cagliari, D. et al. Management of Pest Insects and Plant Diseases by Non-Transformative RNAi. Frontiers in Plant Science vol. 10 (2019). Zotti, M. et al. RNA interference technology in crop protection against arthropod pests, pathogens and nematodes. Pest Management Science vol. 74 1239-1250 (2018). Vallazza, B. et al. Recombinant messenger RNA technology and its application in cancer immunotherapy, transcript replacement therapies, pluripotent stem cell induction, and beyond. Wiley Interdisciplinary Reviews: RNA vol. 6 471-499 (2015). Sahin, U., Karikó, K. & Türeci, Ö. mRNA-based therapeutics-developing a new class of drugs. Nature Reviews Drug Discovery vol. 13 759-780 (2014). Pardi, N., Hogan, M. J. & Weissman, D. Recent advances in mRNA vaccine technology. Current Opinion in Immunology vol. 65 14-20 (2020)). In order to perform the assays required for further elucidating these functions and in order to provide RNA-based active ingredients for application, there is a need for techniques that are capable of producing large quantities of a given RNA.

One of the most straightforward ways to generate RNA is RNA in vitro transcription. This approach is based on imitation of the enzymatic processes that govern RNA synthesis in all forms of life. A common system used for this purpose is the T7 RNA polymerase that, starting from a DNA template, can yield up to milligram quantities of RNA in a few hours of reaction time. However, not all DNA sequences are equally suitable for transcription via the T7 polymerase. In addition, the problem of unspecific addition of nucleotides to the 3′ end results in inhomogeneity that can be crucial when examining regulatory functions. Besides, this method is also labor-intensive. Moreover, scalability of the in vitro transcription reaction and thus maximal achievable amounts of a given RNA are limited, making it challenging and costly to provide a given RNA in large quantities in reasonable time.

Chemical synthesis is currently commonly used for the production of RNA oligonucleotides of less than 10 nucleotides and up to 80 nucleotides. Synthesis is performed on solid supports such as polystyrene or controlled-pore glass and involves addition of the respective nucleotides in 3′ to 5′ direction. Because reaction conditions can be optimized, chemical synthesis allows the production of any given RNA irrespective of its sequence. However, the method is per se not suitable for producing larger RNA molecules having more than 100 nucleotides and is also costly.

Instead, large RNA molecules could be effectively produced by recombinant expression systems, similar to industrial production of recombinant proteins. E. coli has been used to this end, but not without difficulties: Degradation by intracellular RNases, large 3′-end and 5′-end heterogeneity of the transcripts and low RNA-titers have hampered extensive application (Ponchon L, Dardel F. Recombinant RNA technology: the tRNA scaffold. Nat Methods. 2007; 4:571-6). Alternative hosts may offer a better solution (Suzuki H, Ando T, Umekage S, Tanaka T, Kikuchi Y. Extracellular production of an RNA aptamer by ribonuclease-free marine bacteria harboring engineered plasmids: a proposal for industrial RNA drug production. Appl Environ Microbiol. 2010; 76:786-93). However, up to now, they have not been developed yet to a stage where they provide a viable alternative to existing systems (Baronti, L., Karlsson, H., Marušič, M. et al. A guide to large-scale RNA sample preparation. Anal Bioanal Chem. 2018:410, 3239-3252).

### Objective Technical Problem

In order to optimize recombinant RNA expression systems, there is a need for a fast and reliable method for identifying cells that show a high expression of a given RNA molecule.

### SUMMARY OF THE INVENTION

The problem is solved by a method for optimizing the production of a heterologous RNA sequence of interest in a cell, comprising the steps of:
a) introducing into a plurality of cells a vector capable of expressing a heterologous RNA of interest, wherein said RNA is tagged with a RNA tag comprising
   an aptamer capable of stabilizing a fluorophore and
   a scaffold capable of stabilizing the aptamer;
b) culturing the cells in a culture medium under conditions that allow expression of the heterologous RNA of interest;
c) adding said fluorophore to the culture medium;
d) identifying those cells that show the highest intensity of fluorescence and therefore a high expression of the RNA of interest.

Likewise, the problem is solved by a method for producing a heterologous RNA of interest, comprising the steps of
a) introducing into a plurality of cells a vector capable of expressing a heterologous RNA of interest, wherein said RNA is tagged with a RNA tag comprising an aptamer capable of stabilizing a fluorophore and a scaffold capable of stabilizing the aptamer;
b) culturing the cells in a culture medium under conditions that allow expression of the heterologous RNA of interest;
c) adding said fluorophore to the culture medium;
d) identifying and isolating those cells that show the highest intensity of fluorescence and therefore a high expression of the RNA of interest;
e) removing the first vector of step a) from the cells isolated in step d);
f) introducing a second vector capable of expressing the heterologous RNA of interest without the RNA tag into the cells obtained in step e);
g) producing the RNA of interest by culturing the cells obtained in step f).

The problem is also solved by a method for comparing the production capacity of different cells for a heterologous RNA sequence of interest, comprising the steps of:
a) introducing into a plurality of cells a vector capable of expressing a heterologous RNA of interest, wherein said RNA is tagged with a RNA tag comprising
   an aptamer capable of stabilizing a fluorophore and
   a scaffold capable of stabilizing the aptamer;
b) culturing the cells in a culture medium under conditions that allow expression of the RNA of interest;
c) adding said fluorophore to the culture medium;
   comparing the intensity of fluorescence between the plurality of cells.

Furthermore, the problem is solved by a microbial cell harboring a vector capable of expressing a heterologous RNA of interest, wherein said RNA is tagged with a RNA tag comprising
an aptamer capable of stabilizing a fluorophore and
an RNA scaffold capable of stabilizing the aptamer.

### BRIEF DESCRIPTION OF THE FIGURES

FIG. 1 shows the cytometric acquisition of the forward-scatter characteristics (x-axis) and 530 nm fluorescence characteristics (y-axis) of 100,000 representative cells of C. glutamicum ATCC 13032 Δcg2273 pJC1-PF1-U1A-F30::broccoli/UUCG-TF1 without (left panel) and with (right panel) N-Methyl-N′-nitro-N-nitrosoguanidine (MNNG) treatment. The rectangle represents the sorting gate used for the isolation of highly fluorescent cells. (see Example 1d).

FIG. 2 shows the x-fold increase of fluorescence of strain C. glutamicum ATCC 13032_Δcg2273_16S rRNA-broccoli after the addition of the fluorophore DFHBI. The depicted fluorescent induction was recorded by cytometric analysis as median fluorescence (488ex/530-30em) and normalized by the median fluorescence (488ex/530-30em) of the unstained culture of strain C. glutamicum ATCC 13032_Δcg2273_16S rRNA-broccoli.

FIG. 3 shows the relative transcript level of F30::broccoli in strain C. glutamicum ATCC 13032_Δcg2273_16S rRNA-broccoli compared to the control strain C. glutamicum ATCC 13032_Δcg2273 determined by reverse transcription quantitative PCR.

FIG. 4 shows the x-fold increase of fluorescence of strains C. glutamicum ATCC 13032_Δcg2273_pJC1_PF1-U1A-TF1, C. glutamicum ATCC 13032_Δcg2273_pJC1_PF1-U1A-F30::broccoli/UUCG-TF1 and C. glutamicum ATCC 13032_Δcg2273_pJC1_PF1-U1A-F30::mango3-TF1 after the addition of the fluorophore DFHBI or TO1. The depicted fluorescent induction was recorded by cytometric analysis as median fluorescence (488ex/530-30em) and normalized by the median fluorescence (488ex/530-30em) of the unstained cultures, respectively.

FIG. 5 shows the design principle of the DNA construct expressing the fusion of αtubulin senseRNA with F30::broccoli and the corresponding αtubulin antisenseRNA under control of the T7 promoter. Additionally, the scheme of the resulting dsRNA and the corresponding sequence lengths are shown. The arrow represents the activity of RNase A, leading to the fragment visible in FIG. 7, L3.

FIG. 6 shows the x-fold increase of fluorescence of strains C. glutamicum ATCC 13032_Δcg2273_pJC1 and C. glutamicum ATCC 13032_Δcg2273_pJC1_dsRNA_PT7-αtubulin-F30::broccoli after the addition of the fluorophore DFHBI. The depicted fluorescent induction was recorded by cytometric analysis as median fluorescence (488ex/530-30em) and normalized by the median fluorescence (488ex/530-30em) of the unstained cultures, respectively.

FIG. 7 shows a digital gel representation of a capillary electrophoresis analysis of dsRNA ladder (New England Biolabs, Ipswich, MA, USA), C. glutamicum ATCC 13032(DE3)_Δcg2273 pJC1 total RNA (L1), C. glutamicum ATCC 13032(DE3)_Δcg2273 pJC1_dsRNA_PT7-αtubulin-F30::broccoli total RNA (L2) and C. glutamicum ATCC 13032(DE3)_Δcg2273 pJC1_dsRNA_PT7-αtubulin-F30::broccoli total RNA treated with RNase A (L3). The analysis was run on a Fragment Analyzer system equipped with a DNF-471 RNA kit (Agilent Technologies, Santa Clara, CA, USA). LM depicts the lower marker of the DNF-471 RNA kit.

FIG. 8 shows the x-fold increase of fluorescence of strains C. glutamicum ATCC 13032_Δcg2273_pJC1, C. glutamicum ATCC 13032_Δcg2273_pJC1_PT7-luc2(Et)-broccoli-TT7 and C. glutamicum ATCC 13032_Δcg2273_pJC1_PT7-egfp-broccoli-TT7 after the addition of the fluorophore DFHBI. The depicted fluorescent induction was recorded by cytometric analysis as median fluorescence (488ex/530-30em) and normalized by the median fluorescence (488ex/530-30em) of the unstained cultures, respectively.

FIG. 9 shows the in vivo produced RNA fragments egfp-F30::broccoli and luc2-F30::broccoli on an 1% agarose gel. Prepared RNA samples of strains C. glutamicum ATCC 13032(DE3)_Δcg2273_pJC1-PT7-egfp-broccoli-TT7 and C. glutamicum ATCC 13032(DE3)_Δcg2273_pJC1-PT7-uc2-broccoli-TT7 were used to reverse transcribe RNA into cDNA by ProtoScriptII reverse transcriptase followed by the amplification of the fragments egfp-F30::broccoli and luc2-F30::broccoli by OneTaq Hot Start DNA polymerase and appropriate primers (lanes 3 and 6). As positive control, prepared plasmid DNA was amplified using same DNA polymerase and primers (lanes 1 and 4) and as negative control, prepared RNA samples were amplified using same DNA polymerase and primers without reverse transcribing RNA into cDNA (lane 2 and 5).

### DETAILED DESCRIPTION

As used herein, the terms “comprise” and “comprising” are understood to mean both “contain/containing” and “consist/consisting”.

In one aspect, the invention is directed to a method for optimizing the production of a heterologous RNA sequence of interest in a cell, comprising the steps of:
a) introducing into a plurality of cells a vector capable of expressing a heterologous RNA of interest, wherein said RNA is tagged with a RNA tag comprising
   an aptamer capable of stabilizing a fluorophore and
   a scaffold capable of stabilizing the aptamer;
b) culturing the cells in a culture medium under conditions that allow expression of the heterologous RNA of interest;
c) adding said fluorophore to the culture medium;
d) identifying those cells that show the highest intensity of fluorescence and therefore a high expression of the RNA of interest.

In the context of the invention, a cell can be a eukaryotic or a prokaryotic cell. In one embodiment, the cell is a microbial cell. Microbial cells or microbes are useful for producing molecules such as DNA, RNA or proteins. They can be differentiated into Gram-positive and Gram-negative microbes. Preferably, the microbial cells according to the invention are Gram positive microbial cells. In one aspect of the invention, the microbial cells according to the invention are from the genus of Corynebacterium, in particular Corynebacterium glutamicum. The cells according to and used in the invention are herein also referred to as host cells.

First, a vector capable of expressing a heterologous RNA of interest is introduced into the cells. The vector can be any vector suitable for expressing a RNA in a host cell, such as a plasmid; a viral vector, e.g. a retrovirus, lentivirus, adenovirus, adeno-associated virus or Lambda phage; or an artificial chromosome, e.g. a BAC, YAC or HAC.

The vector used in the invention is capable of expressing an RNA of interest. The expression of the RNA of interest can be constitutive or conditional. For example, expression of the RNA of interest may be induced by addition of acetate, anhydrotetracycline, arabinose, gluconate, isopropyl β-D-1-thiogalactopyranoside (IPTG), light, maltose, methanol, propionate or by increasing the cultivation temperature.

Other than the elements listed below, the vector may comprise additional elements that are necessary for or enhance expression, molecular cloning and replication. For example, the vector may comprise selection or marker genes such as lacZ encoding beta-galactosidase, luc encoding luciferase, cat encoding chloramphenicol transferase or other resistance genes conveying resistance to antibiotics. The vector may also comprise an origin of replication, a multiple cloning site and/or transcriptional terminators.

The RNA of interest that is expressed by the vector is a heterologous RNA, i.e. an RNA molecule that is not expressed in the wildtype of the host cell. The sequence of the RNA of interest is not limited and can be any naturally or artificially occurring RNA sequence. In one embodiment, the RNA of interest is a mRNA, viral RNA, retroviral RNA, antisense RNA, replicon RNA, bicistronic or multicistronic RNA, small interfering RNA or immunostimulating RNA. In one embodiment, the RNA of interest has a length of 20-10000 nucleotides.

The RNA of interest is tagged with an RNA tag. “Tagged” herein means that the RNA tag is linked to the RNA of interest and that the tag is expressed together with the RNA of interest. Upon expression of the heterologous RNA from the vector, the RNA tag is not removed from the RNA of interest. According to the invention, it is not necessary that the tag is directly attached to the RNA of interest, although this is a preferred embodiment. But the RNA tag and the RNA of interest may also be separated by a nucleotide spacer sequence.

The RNA tag used in the invention comprises an aptamer. An “aptamer” is herein understood to refer to a RNA oligonucleotide that is capable of binding a small molecule fluorophore. Usually, aptamers are 10-100 base nucleic acid oligonucleotides that bind with high affinity to small molecules to induce their fluorescence. Before a binding event occurs, neither the aptamer nor the target small molecule are strongly fluorescent but the binding of an aptamer to its target molecule activates the fluorescence of the target molecule. Aptamers capable of binding a molecule of interest can be generated via systematic evolution of ligands by exponential enrichment (SELEX) (Paige, J. S., Wu, K. Y., and Jaffrey, S. R. RNA mimics of green fluorescent protein. Science; 2011; 333, 642-6.). A variety of aptamers have been described in literature (Ouellet, J. (2016) RNA fluorescence with light-Up aptamers. Front. Chem. 4, 1-12. Bouhedda, F., Autour, A., and Ryckelynck, M. (2017) Light-Up RNA Aptamers and Their Cognate Fluorogens: From Their Development to Their Applications. Int. J. Mol. Sci. 19, 44.). Preferred aptamer sequences to be used in the invention include:

Aptamer II-mini3-4:
SEQ ID NO: 1
AGGUUCGAAGCUUUUGCUUGGACGAACCG
Mango:
SEQ ID NO: 2
GAAGGGACGGUGCGGAGAGGAGA
Mango2:
SEQ ID NO: 3
GAAGGAGAGGAGAGGAAGAGGAGA
Mango3:
SEQ ID NO: 4
GGAAGGAUUGGUAUGUGGUAUA
Mango4:
SEQ ID NO: 5
CGAGGGAGUGGUGAGGAUGAGGCGA
Spinach:
SEQ ID NO: 6
GACGCAACUGAAUGAAAUGGUGAAGGACGGGUCCAGGUGUGGCUGCUU
CGGCAGUGCAGCUUGUUGAGUAGAGUGUGAGCUCCGUAACUAGUCGCG
UC
Spinach2:
SEQ ID NO: 7
GAUGUAACUGAAUGAAAUGGUGAAGGACGGGUCCAGUAGGCUGCUUCG
GCAGCCUACUUGUUGAGUAGAGUGUGAGCUCCGUAACUAGUUACAUC
Broccoli:
SEQ ID NO: 8
AGACGGUCGGGUCCAGAUAUUCGUAUCUGUCGAGUAGAGUGUGGGCU
Corn:
SEQ ID NO: 13
CGAGGAAGGAGGUCUGAGGAGGUCACUG
Orange broccoli:
SEQ ID NO: 14
GAGACGCAACUGAAUGAAAUGGUGAAGGAGACGGUCGGGUCCAGGUGC
ACAAAUGUGGCCUGUUGAGUAGCGUGUGGGCUCCGUAACUAGUCGCGU
CAC
Orange broccoli short version:
SEQ ID NO: 65
AGACGGUCGGGUCCAGGUGCACAAAUGUGGCCUGUUGAGUAGCGUGUG
GGCU
Red broccoli:
SEQ ID NO: 15
GAGACGCAACUGAAUGAAAUGUUUUCGGAGACGGUCGGGUCCAGUCCC
AACGAUGUUGGCUGUUGAGUAGUGUGUGGGCUCCGUAACUAGUCGCGU
CAC
Red broccoli short version:
SEQ ID NO: 66
AGACGGUCGGGUCCAGUCCCAACGAUGUUGGCUGUUGAGUAGUGUGUG
GGCU
Baby spinach:
SEQ ID NO: 16
AAGGACGGGUCCGUUGAGUAGAGUGUGAG

The aptamers contained within the RNA tag used in the invention are capable of binding a fluorophore, i.e. an organic molecule that emits fluorescence upon light excitation. Light emission intensity essentially depends on binding of the fluorophore to the aptamer because the fluorophore's structure is stabilized when bound to the aptamer. This stabilization results in a preferred dissociation of excitation energy as fluorescence. That means that according to the invention, the fluorophores light emission strongly increases after binding to an aptamer.

The fluorophores used in the invention are small, non-toxic molecules that can easily enter into a cell. Pairs of aptamers and fluorophores that can bind to these aptamers have been previously described. Fluorophores that can be bound by aptamers include (Z)-4-(2-hydroxybenzylidene)-1,2-dimethyl-1H-imidazol-5(4H)-one (2-HBI), (5Z)-5-[(3,5-Difluoro-4-hydroxyphenyl)methylene]-3,5-dihydro-2,3-dimethyl-4H-imidazol-4-one (DFHBI), (5)-5-[(3,5-Difluoro-4-hydroxyphenyl)nethylene]-3,5-dihydro-2-methyl-3-(2,2,2-trifluoroethyl)-4H-imidazol-4-one (DFHBI-1T), DFHBI-2T, 2-(4-(dimethylamino)benzylidene)-1H-indene-1,3(2H)-dione (DMABI), (Z)-4-(3,5-dimethyl-4-hydroxybenzylidene)-1,2-dimethyl-1H-imidazol-5(4H)-one (DMHBI), 3,5-difluoro-4-hydroxybenzylidene imidazolinone-2-oxime (DFHO), 2-(2-Methylbenzo[d]thiazol-3-ium-3-yl)acetate (TO1), 4-[(E)-2-(acetylphenylamino)ethenyl]-1-methylquinolinium iodide (TO3) and (N-(6-aminohexyl)-2-(2,6-di-tert-butyl-4-(5-(4-methylpiperazin-1-yl)-1H,1′H-2,5′-bibenzo[d]imidazol-2′-yl)phenoxy)acetamide) (Hoechst 1C).

In a preferred embodiment, the aptamer comprises SEQ ID NO: 1 and the fluorophore is Hoechst 1C. In another preferred embodiment, the aptamer comprises SEQ ID NO: 2 and the fluorophore is TO1 or TO3. In another preferred embodiment, the aptamer comprises SEQ ID NO: 3 and the fluorophore is TO1 or TO3. In another preferred embodiment, the aptamer comprises SEQ ID NO: 4 and the fluorophore is TO1 or TO3. In another preferred embodiment, the aptamer comprises SEQ ID NO: 5 and the fluorophore is TO1 or TO3. In another preferred embodiment, the aptamer comprises SEQ ID NO: 6 and the fluorophore is any of 2-HBI, DFHBI, DFHBI-1T, DFHBI-2T, DMABI or DMHBI. In another preferred embodiment, the aptamer comprises SEQ ID NO: 7 and the fluorophore is any of 2-HBI, DFHBI, DFHBI-1T, DFHBI-2T, DMABI or DMHBI. In another preferred embodiment, the aptamer comprises SEQ ID NO: 8 and the fluorophore is any of 2-HBI, DFHBI, DFHBI-1T, DFHBI-2T, DMABI or DMHBI. In another preferred embodiment, the aptamer comprises SEQ ID No: 13, 14, 15, 65 or 66 and the fluorophore is DFHO. In another preferred embodiment, the aptamer comprises SEQ ID No: 16 and the fluorophore is DHFBI or DHFBI-1T.

The RNA tag used in the invention further comprises an RNA scaffold. The RNA scaffold can be any nucleotide sequence that is capable of stabilizing the aptamer, supporting the formation of the functional aptamer structure and reducing aptamer degradation. A RNA scaffold according to the invention comprises at least one insertion site represented by NNNN. In the vectors used in the invention, NNNN is replaced by the respective aptamer.

In one embodiment, the scaffold comprises two insertion sites, e.g. as in SEQ ID NO: 11 and 12. According to the invention, one or two aptamers may be inserted into such a scaffold. If two aptamers are inserted, the same or different aptamers may be inserted into the two insertion sites. Examples for this are SEQ ID NO: 77 and 78. If the same aptamer is inserted into both insertion sites, the fluorescence signal emitted after addition of the fluorophore will be stronger than if only one aptamer is present. Using two different aptamers has the advantage that cells expressing the RNA of interest will emit two different fluorescence signals. This may be beneficial to exclude false positives.

If only one aptamer is inserted into scaffolds having two insertion sites, the second insertion site may be replaced with any spacer sequence. In a preferred embodiment, the spacer sequences comprises four nucleotides. In a particularly preferred embodiment, the spacer sequence is UUCG or TTCG.

In a preferred embodiment, the RNA scaffold comprises one of the following sequences:

tRNA^{Lys}:
SEQ ID NO: 9
GCCCGGAUAGCUCAGUCGGUAGAGCAGNNNNCGGGUCCAGGGUUCAAGUCCCUGUUCGGGCGCCA
V5:
SEQ ID NO: 10
UGCCUGGCGACCAUAGCGAUUGNNNNCAAUUAGCGCCGAUGGUAGUGUGGGGUUUCCCCAUGUGAGAGUAGGACAUCGCCAGGCAU
F29:
SEQ ID NO: 11
UUGUCACGUGUAUGUGGGNNNNCCCACAUACUUUGUUGAUCCNNNNGGAUCAAUCAUGGCAA
F30:
SEQ ID NO: 12
UUGCCAUGUGUAUGUGGGNNNNCCCACAUACUCUGAUGAUCCNNNNGGAUCAUUCAUGGCAA
In one embodiment, the RNA tag comprises any of SEQ ID NO: 17 to 64, 69, 77 or 78.
In a preferred embodiment, the RNA tag comprises SEQ ID NO: 69.
tRNALys::Aptamer II-mini3-4
SEQ ID NO: 17
GCCCGGAUAGCUCAGUCGGUAGAGCAGAGGUUCGAAGCUUUUGCUUGGACGAAC
CGCGGGUCCAGGGUUCAAGUCCCUGUUCGGGCGCCA
tRNALys::Mango
SEQ ID NO: 18
GCCCGGAUAGCUCAGUCGGUAGAGCAGGAAGGGACGGUGCGGAGAGGAGACGGG
UCCAGGGUUCAAGUCCCUGUUCGGGCGCCA
tRNALys::Mango2
SEQ ID NO: 19
GCCCGGAUAGCUCAGUCGGUAGAGCAGGAAGGAGAGGAGAGGAAGAGGAGACGG
GUCCAGGGUUCAAGUCCCUGUUCGGGCGCCA
tRNALys::Mango3
SEQ ID NO: 20
GCCCGGAUAGCUCAGUCGGUAGAGCAGGGAAGGAUUGGUAUGUGGUAUACGGGU
CCAGGGUUCAAGUCCCUGUUCGGGCGCCA
tRNALys: Mango4
SEQ ID NO: 21
GCCCGGAUAGCUCAGUCGGUAGAGCAGCGAGGGAGUGGUGAGG
AUGAGGCGACGGGUCCAGGGUUCAAGUCCCUGUUCGGGCGCCA
tRNALys::Spinach
SEQ ID NO: 22
GCCCGGAUAGCUCAGUCGGUAGAGCAGGACGCAACUGAAUGAAAUGGUGAAGGAC
GGGUCCAGGUGUGGCUGCUUCGGCAGUGCAGCUUGUUGAGUAGAGUGUGAGCUC
CGUAACUAGUCGCGUCCGGGUCCAGGGUUCAAGUCCCUGUUCGGGCGCCA
tRNALys::Spinach2
SEQ ID NO: 23
GCCCGGAUAGCUCAGUCGGUAGAGCAGGAUGUAACUGAAUGAAAUGGUGAAGGAC
GGGUCCAGUAGGCUGCUUCGGCAGCCUACUUGUUGAGUAGAGUGUGAGCUCCGU
AACUAGUUACAUCCGGGUCCAGGGUUCAAGUCCCUGUUCGGGCGCCA
tRNALys::Broccoli
SEQ ID NO: 24
GCCCGGAUAGCUCAGUCGGUAGAGCAGAGACGGUCGGGUCCAGAUAUUCGUAUC
UGUCGAGUAGAGUGUGGGCUCGGGUCCAGGGUUCAAGUCCCUGUUCGGGCGCCA
tRNALys::Corn
SEQ ID NO: 25
GCCCGGAUAGCUCAGUCGGUAGAGCAGCGAGGAAGGAGGUCUGAGGAGGUCACU
GCGGGUCCAGGGUUCAAGUCCCUGUUCGGGCGCCA
tRNALys::Orange broccoli
SEQ ID NO: 26
GCCCGGAUAGCUCAGUCGGUAGAGCAGAGACGGUCGGGUCCAGGUGCACAAAUG
UGGCCUGUUGAGUAGCGUGUGGGCUCGGGUCCAGGGUUCAAGUCCCUGUUCGGG
CGCCA
tRNALys::Red broccoli
SEQ ID NO: 27
GCCCGGAUAGCUCAGUCGGUAGAGCAGAGACGGUCGGGUCCAGUCCCAACGAUG
UUGGCUGUUGAGUAGUGUGUGGGCUCGGGUCCAGGGUUCAAGUCCCUGUUCGGG
CGCCA
tRNALys::Baby spinach
SEQ ID NO: 28
GCCCGGAUAGCUCAGUCGGUAGAGCAGAAGGACGGGUCCGUUGAGUAGAGUGUG
AGCGGGUCCAGGGUUCAAGUCCCUGUUCGGGCGCCA
V5::Aptamer II-mini3-4
SEQ ID NO: 29
UGCCUGGCGACCAUAGCGAUUGAGGUUCGAAGCUUUUGCUUGGACGAACCGCAAU
UAGCGCCGAUGGUAGUGUGGGGUUUCCCCAUGUGAGAGUAGGACAUCGCCAGGC
AU
V5::Mango
SEQ ID NO: 30
UGCCUGGCGACCAUAGCGAUUGGAAGGGACGGUGCGGAGAGGAGACAAUUAGCG
CCGAUGGUAGUGUGGGGUUUCCCCAUGUGAGAGUAGGACAUCGCCAGGCAU
V5::Mango2
SEQ ID NO: 31
UGCCUGGCGACCAUAGCGAUUGGAAGGAGAGGAGAGGAAGAGGAGACAAUUAGCG
CCGAUGGUAGUGUGGGGUUUCCCCAUGUGAGAGUAGGACAUCGCCAGGCAU
V5::Mango3
SEQ ID NO: 32
UGCCUGGCGACCAUAGCGAUUGGGAAGGAUUGGUAUGUGGUAUACAAUUAGCGC
CGAUGGUAGUGUGGGGUUUCCCCAUGUGAGAGUAGGACAUCGCCAGGCAU
V5::Mango4
SEQ ID NO: 33
UGCCUGGCGACCAUAGCGAUUGCGAGGGAGUGGUGAGGAUGAGGCGACAAUUAG
CGCCGAUGGUAGUGUGGGGUUUCCCCAUGUGAGAGUAGGACAUCGCCAGGCAU
V5::Spinach
SEQ ID NO: 34
UGCCUGGCGACCAUAGCGAUUGGACGCAACUGAAUGAAAUGGUGAAGGACGGGUC
CAGGUGUGGCUGCUUCGGCAGUGCAGCUUGUUGAGUAGAGUGUGAGCUCCGUAA
CUAGUCGCGUCCAAUUAGCGCCGAUGGUAGUGUGGGGUUUCCCCAUGUGAGAGU
AGGACAUCGCCAGGCAU
V5::Spinach2
SEQ ID NO: 35
UGCCUGGCGACCAUAGCGAUUGGAUGUAACUGAAUGAAAUGGUGAAGGACGGGUC
CAGUAGGCUGCUUCGGCAGCCUACUUGUUGAGUAGAGUGUGAGCUCCGUAACUA
GUUACAUCCAAUUAGCGCCGAUGGUAGUGUGGGGUUUCCCCAUGUGAGAGUAGG
ACAUCGCCAGGCAU
V5::Broccoli
SEQ ID NO: 36
UGCCUGGCGACCAUAGCGAUUGAGACGGUCGGGUCCAGAUAUUCGUAUCUGUCG
AGUAGAGUGUGGGCUCAAUUAGCGCCGAUGGUAGUGUGGGGUUUCCCCAUGUGA
GAGUAGGACAUCGCCAGGCAU
V5::Corn
SEQ ID NO: 37
UGCCUGGCGACCAUAGCGAUUGCGAGGAAGGAGGUCUGAGGAGGUCACUGCAAU
UAGCGCCGAUGGUAGUGUGGGGUUUCCCCAUGUGAGAGUAGGACAUCGCCAGGC
AU
V5::Orange broccoli
SEQ ID NO: 38
UGCCUGGCGACCAUAGCGAUUGAGACGGUCGGGUCCAGGUGCACAAAUGUGGCC
UGUUGAGUAGCGUGUGGGCUCAAUUAGCGCCGAUGGUAGUGUGGGGUUUCCCCA
UGUGAGAGUAGGACAUCGCCAGGCAU
V5::Red broccoli
SEQ ID NO: 39
UGCCUGGCGACCAUAGCGAUUGAGACGGUCGGGUCCAGUCCCAACGAUGUUGGC
UGUUGAGUAGUGUGUGGGCUCAAUUAGCGCCGAUGGUAGUGUGGGGUUUCCCCA
UGUGAGAGUAGGACAUCGCCAGGCAU
V5::Baby spinach
SEQ ID NO: 40
UGCCUGGCGACCAUAGCGAUUGAAGGACGGGUCCGUUGAGUAGAGUGUGAGCAA
UUAGCGCCGAUGGUAGUGUGGGGUUUCCCCAUGUGAGAGUAGGACAUCGCCAGG
CAU
F29::Aptamer II-mini3-4
SEQ ID NO: 41
UUGUCACGUGUAUGUGGGAGGUUCGAAGCUUUUGCUUGGACGAACCGCCCACAU
ACUUUGUUGAUCCNNNNGGAUCAAUCAUGGCAA
F29::Mango
SEQ ID NO: 42
UUGUCACGUGUAUGUGGGGAAGGGACGGUGCGGAGAGGAGACCCACAUACUUUG
UUGAUCCNNNNGGAUCAAUCAUGGCAA
F29::Mango2
SEQ ID NO: 43
UUGUCACGUGUAUGUGGGGAAGGAGAGGAGAGGAAGAGGAGACCCACAUACUUU
GUUGAUCCNNNNGGAUCAAUCAUGGCAA
F29::Mango3
SEQ ID NO: 44
UUGUCACGUGUAUGUGGGGGAAGGAUUGGUAUGUGGUAUACCCACAUACUUUGU
UGAUCCNNNNGGAUCAAUCAUGGCAA
F29::Mango4
SEQ ID NO: 45
UUGUCACGUGUAUGUGGGCGAGGGAGUGGUGAGGAUGAGGCGACCCACAUACUU
UGUUGAUCCNNNNGGAUCAAUCAUGGCAA
F29::Spinach
SEQ ID NO: 46
UUGUCACGUGUAUGUGGGGACGCAACUGAAUGAAAUGGUGAAGGACGGGUCCAG
GUGUGGCUGCUUCGGCAGUGCAGCUUGUUGAGUAGAGUGUGAGCUCCGUAACUA
GUCGCGUCCCCACAUACUUUGUUGAUCCNNNNGGAUCAAUCAUGGCAA
F29::Spinach2
SEQ ID NO: 47
UUGUCACGUGUAUGUGGGGAUGUAACUGAAUGAAAUGGUGAAGGACGGGUCCAG
UAGGCUGCUUCGGCAGCCUACUUGUUGAGUAGAGUGUGAGCUCCGUAACUAGUU
ACAUCCCCACAUACUUUGUUGAUCCNNNNGGAUCAAUCAUGGCAA
F29::Broccoli
SEQ ID NO: 48
UUGUCACGUGUAUGUGGGAGACGGUCGGGUCCAGAUAUUCGUAUCUGUCGAGUA
GAGUGUGGGCUCCCACAUACUUUGUUGAUCCNNNNGGAUCAAUCAUGGCAA
F29::Corn
SEQ ID NO: 49
UUGUCACGUGUAUGUGGGCGAGGAAGGAGGUCUGAGGAGGUCACUGCCCACAUA
CUUUGUUGAUCCNNNNGGAUCAAUCAUGGCAA
F29::Orange broccoli
SEQ ID NO: 50
UUGUCACGUGUAUGUGGGAGACGGUCGGGUCCAGGUGCACAAAUGUGGCCUGUU
GAGUAGCGUGUGGGCUCCCACAUACUUUGUUGAUCCNNNNGGAUCAAUCAUGGCA
A
F29::Red broccoli
SEQ ID NO: 51
UUGUCACGUGUAUGUGGGAGACGGUCGGGUCCAGUCCCAACGAUGUUGGCUGUU
GAGUAGUGUGUGGGCUCCCACAUACUUUGUUGAUCCNNNNGGAUCAAUCAUGGCA
A
F29::Baby spinach
SEQ ID NO: 52
UUGUCACGUGUAUGUGGGAAGGACGGGUCCGUUGAGUAGAGUGUGAGCCCACAU
ACUUUGUUGAUCCNNNNGGAUCAAUCAUGGCAA
F30::AptamerII-mini3-4
SEQ ID NO: 53
UUGCCAUGUGUAUGUGGGAGGUUCGAAGCUUUUGCUUGGACGAACCGCCCACAU
ACUCUGAUGAUCCNNNNGGAUCAUUCAUGGCAA
F30::Mango:
SEQ ID NO: 54
UUGCCAUGUGUAUGUGGGGAAGGGACGGUGCGGAGAGGAGACCCACAUACUCUG
AUGAUCCNNNNGGAUCAUUCAUGGCAA
F30::Mango2:
SEQ ID NO: 55
UUGCCAUGUGUAUGUGGGGAAGGAGAGGAGAGGAAGAGGAGACCCACAUACUCU
GAUGAUCCNNNNGGAUCAUUCAUGGCAA
F30::Mango3:
SEQ ID NO: 56
UUGCCAUGUGUAUGUGGGGGAAGGAUUGGUAUGUGGUAUACCCACAUACUCUGA
UGAUCCNNNNGGAUCAUUCAUGGCAA
F30::Mango4
SEQ ID NO: 57
UUGCCAUGUGUAUGUGGGCGAGGGAGUGGUGAGGAUGAGGCGACCCACAUACUC
UGAUGAUCCNNNNGGAUCAUUCAUGGCAA
F30::Spinach
SEQ ID NO: 58
UUGCCAUGUGUAUGUGGGGACGCAACUGAAUGAAAUGGUGAAGGACGGGUCCAG
GUGUGGCUGCUUCGGCAGUGCAGCUUGUUGAGUAGAGUGUGAGCUCCGUAACUA
GUCGCGUCCCCACAUACUCUGAUGAUCCNNNNGGAUCAUUCAUGGCAA
F30::Spinach2
SEQ ID NO: 59
UUGCCAUGUGUAUGUGGGGAUGUAACUGAAUGAAAUGGUGAAGGACGGGUCCAG
UAGGCUGCUUCGGCAGCCUACUUGUUGAGUAGAGUGUGAGCUCCGUAACUAGUU
ACAUCCCCACAUACUCUGAUGAUCCNNNNGGAUCAUUCAUGGCAA
F30::Broccoli
SEQ ID NO: 60
UUGCCAUGUGUAUGUGGGAGACGGUCGGGUCCAGAUAUUCGUAUCUGUCGAGUA
GAGUGUGGGCUCCCACAUACUCUGAUGAUCCNNNNGGAUCAUUCAUGGCAA
F30::Corn
SEQ ID NO: 61
UUGCCAUGUGUAUGUGGGCGAGGAAGGAGGUCUGAGGAGGUCACUGCCCACAUA
CUCUGAUGAUCCNNNNGGAUCAUUCAUGGCAA
F30::Orange broccoli
SEQ ID NO: 62
UUGCCAUGUGUAUGUGGGAGACGGUCGGGUCCAGGUGCACAAAUGUGGCCUGUU
GAGUAGCGUGUGGGCUCCCACAUACUCUGAUGAUCCNNNNGGAUCAUUCAUGGCA
A
F30::Red broccoli
SEQ ID NO: 63
UUGCCAUGUGUAUGUGGGAGACGGUCGGGUCCAGUCCCAACGAUGUUGGCUGUU
GAGUAGUGUGUGGGCUCCCACAUACUCUGAUGAUCCNNNNGGAUCAUUCAUGGCA
A
F30::Baby spinach
SEQ ID NO: 64
UUGCCAUGUGUAUGUGGGAAGGACGGGUCCGUUGAGUAGAGUGUGAGCCCACAU
ACUCUGAUGAUCCNNNNGGAUCAUUCAUGGCAA
F30-Broccoli-Broccoli
SEQ ID NO: 77
UUGCCAUGUGUAUGUGGGAGACGGUCGGGUCCAGAUAUUCGUAUCUGUCGAGUA
GAGUGUGGGCUCCCACAUACUCUGAUGAUCCAGACGGUCGGGUCCAGAUAUUCG
UAUCUGUCGAGUAGAGUGUGGGCUGGAUCAUUCAUGGCAA
F30-Broccoli-Mango
SEQ ID NO: 78
UUGCCAUGUGUAUGUGGGAGACGGUCGGGUCCAGAUAUUCGUAUCUGUCGAGUA
GAGUGUGGGCUCCCACAUACUCUGAUGAUCCGAAGGGACGGUGCGGAGAGGAGA
GGAUCAUUCAUGGCAA

The vector used in the invention can be introduced into the cells by any method known in the art, e.g. by transformation, transfection or viral transduction. The skilled person is aware how these methods can be further optimized to ensure that the vector is present in each cell in sufficient quantity. It is envisaged by the present invention that the vector can be integrated into the chromosome once it has been introduced into the cells. This integration may not necessarily include the complete vector sequence, but the sections of the vector required for expression of the target RNA in the given genetic context.

The method according to the invention aims at optimizing RNA production by identifying cells or culture conditions that increase RNA production. Therefore, in one embodiment, the cells used in the methods of the invention carry chromosomal genetic mutations that may influence RNA expression. The cells can be mutagenized using any technique known in the art, e.g., random mutagenesis via UV radiation or site-directed mutagenesis, for example via CRISPR-Cas.

In another embodiment, the cells harbor different vectors that are all capable of expressing the same RNA of interest, but differ in the other elements contained in the vector, so that the RNA yield from the vectors is different.

Once the vector has been introduced into the cells, the cells are cultured under conditions that allow expression of the heterologous RNA. In case that expression of the heterologous RNA of interest is conditional, the cells may first be cultured without inducing expression and expression be induced after some time.

In one embodiment, culture conditions between the cells are varied, for example with respect to temperature, dissolved oxygen level, stirring speed, pressure or culture medium. This embodiment allows to identify optimal culture conditions for the expression of the heterologous RNA in question. Therefore, the method of the invention can also be used to optimize culture conditions for the production of a particular RNA of interest.

After the cells have been cultured for a time sufficient to express the heterologous RNA from the vector, the fluorophore that is capable of binding the aptamer with which the heterologous RNA is tagged is added to the culture medium and allowed to enter the cells where it can bind to the aptamer. It is known in the art how to determine a suitable concentration of the fluorophore in the culture medium.

In those cells that have a high concentration of the heterologous RNA of interest, the tag is present in higher quantities and thus higher amounts of the fluorophore will be bound and emit fluorescence. In contrast, those cells showing only a low concentration of the RNA of interest including the tag will harbor less activated fluorophore, i.e., fluorophore bound to an aptamer, and therefore emit less fluorescence. Importantly, fluorophore that is present in the cell, but not bound to the aptamer, will exhibit only very weak or no fluorescence.

The degree of fluorescence emitted by each cell can be determined using any technique known in the art. In one embodiment, the cells are assessed by spectrometry. In a preferred embodiment, the cells are sorted according to their fluorescence level by flow cytometry. This allows to identify and, at the same time, isolate those cells showing a high level of fluorescence. Isolated cells may be subsequently analyzed for the chromosomal genetic alterations that they carry or genetic alterations in the vector. Likewise, it is possible to determine those culture conditions that yield the highest number of fluorescent cells.

In another embodiment, the invention relates to a method for comparing the production capacity of different cells for a heterologous RNA sequence of interest, comprising the steps of:
d) introducing into a plurality of cells a vector capable of expressing a heterologous RNA of interest, wherein said RNA is tagged with a RNA tag comprising
   an aptamer capable of stabilizing a fluorophore and
   a scaffold capable of stabilizing the aptamer;
e) culturing the cells in a culture medium under conditions that allow expression of the RNA of interest;
f) adding said fluorophore to the culture medium;
g) comparing the intensity of fluorescence between the plurality of cells.

In another aspect, the invention relates to a method for producing a RNA of interest, comprising the steps of
a) introducing into a plurality of cells a first vector capable of expressing a heterologous RNA of interest, wherein said RNA is tagged with a RNA tag comprising an aptamer capable of stabilizing a fluorophore and a scaffold capable of stabilizing the aptamer;
b) culturing the cells in a culture medium under conditions that allow expression of the heterologous RNA of interest;
c) adding said fluorophore to the culture medium;
d) identifying and isolating those cells that show the highest intensity of fluorescence and therefore a high expression of the RNA of interest;
e) removing the first vector of step a) from the cells isolated in step d);
f) introducing a second vector capable of expressing the heterologous RNA of interest without the RNA tag into the cells obtained in step e);
g) producing the RNA of interest by culturing the cells obtained in step f).

Producing a RNA of interest according to the method of the invention comprises first identifying cells that show a high expression of the RNA of interest with the help of the RNA tag and then using these cells for the production of the RNA of interest.

Because it is desirable that the final product does not include any unnecessary sequences, the vector capable of expressing the tagged RNA of interest is removed prior to RNA production once suitable cells have been identified. Removal of a vector can be achieved by preparation of electrocompetent cells as previously described (Tauch, A., Kirchner, O., Loffler, B., Götker, S., Pühler, A., and Kalinowski, J. Efficient Electrotransformation of Corynebacterium diphtheriae with a Mini-Replicon Derived from the Corynebacterium glutamicum Plasmid pGA1. Curr. Microbiol. 2002; 45, 362-367) and electroporation of these cell without addition of a DNA template. This increases the likelihood of spontaneous vector loss. Cells are subsequently transformed with a second vector that is identical to the first vector except that the second vector does not contain the RNA tag. These cells are then used for producing the RNA of interest.

Extraction and, optionally, purification of the produced RNA can be performed according to methods known in the art. Likewise, the amount of produced RNA can be quantified after extraction using well-known techniques.

The method of the invention allows to maximize the production of the RNA of interest by specifically selecting cells that show a high expression of the RNA of interest. Because the vectors used in the invention can be easily engineered to carry any RNA of interest, the invention provides a fast, efficient and universally applicable way to save costs and time when producing a certain RNA molecule.

In a third aspect, the invention relates to a microbial cell harboring a vector capable of expressing a heterologous RNA of interest, wherein said RNA is tagged with a RNA tag comprising
an aptamer capable of stabilizing a fluorophore and
an RNA scaffold capable of stabilizing the aptamer.

The cells according to the invention harbor a vector capable of expressing a heterologous RNA of interest. “Harboring” is herein defined as meaning that the cells contain or comprise a vector either as an extrachromosomal plasmid or integrated into one or several of their chromosomes.

Because the heterologous RNA that is expressed by the cells of the invention is tagged, it can be detected and quantified once the corresponding fluorophore has been added to the cells. Therefore, cells according to the invention can be easily and conveniently classified and separated (e.g. by flow cytometry) based on the amount of heterologous RNA they produce.

### EXAMPLES

Hereinafter, the present invention is described in more detail with reference to Figures and the Examples, which however are not intended to limit the present invention.

### Example 1

a) Construction of the Vectors pJC1-PF1-U1A-F30::broccoli/UUCG-TF1 and pJC1-PF1-U1A-TF1

The construction of the plasmid vector was achieved by means of chemical synthesis of synthetic DNA-fragments (SEQ ID NO: 72 for pJC1-PF1-U1A-TF1 and SEQ ID NO: 71 for pJC1-PF1-U1A-F30::broccoli/UUCG-TF1) and their ligation into pJC1 (Cremer, J., Treptow, C., Eggeling, L., and Sahm, H. Regulation of Enzymes of Lysine Biosynthesis in Corynebacterium glutamicum. Microbiol. 1988; 134, 3221-3229). SEQ ID NO: 71 contained the promoter P_{F1} (SEQ ID NO: 67), the non-coding, recombinant U1A*-RNA (SEQ ID NO: 68) that was described earlier (Hashiro, S., Mitsuhashi, M., and Yasueda, H. Overexpression system for recombinant RNA in Corynebacterium glutamicum using a strong promoter derived from corynephage BFK20. J. Biosci. Bioeng. 2019; 128, 255-263), the F30 scaffold (SEQ ID NO: 69) with a broccoli aptamer (SEQ ID NO: 8) in the first integration point and a “UUCG spacer” in the second integration point and a terminator sequence T_{F1} (SEQ ID NO: 70). SEQ ID NO: 72 contained the promoter P_{F1} (SEQ ID NO: 67), a non-coding, recombinant U1A*-RNA (SEQ ID NO: 68) and a terminator sequence T_{F1} (SEQ ID NO: 70), but neither scaffold nor aptamer sequence.

After cleavage of the synthesized DNA fragments with the restriction enzymes XbaI and SalI and subsequent purification of the reaction mixtures, the DNA fragments that had been cut out were used in individual ligation reactions with vector pJC1 that had also been linearized with XbaI and SalI and dephosphorylated. The ligation mixtures were used directly to transform E. coli XL1-blue, and the selection of transformants was carried out on LB plates containing 50 pg/ml kanamycin. 16 colonies which grew on these plates and were therefore resistant to kanamycin were used for colony PCR. The colony PCR was performed with primers pJC1_check_f (SEQ ID NO: 73) and pJC1_check_rev (SEQ ID NO: 74), to analyze whether the synthesized fragments were inserted into vector pJC1. The analysis of colony PCR products in an agarose gel showed the expected PCR product with a size of 521 bp (pJC1-PF1-U1A-TF1) and 626 bp (pJC1-PF1-U1A-F30::broccoli/UUCG-TF1) in the samples that were analyzed, whereupon four colonies were cultured for plasmid preparations in a larger scale. After 16 h of cultivation these cultures were collected by centrifugation and plasmid DNA was prepared. Two of the plasmid preparations were sequenced with the primers used in the colony PCR. Sequence analysis of the inserts showed 100% identity with the expected sequence. The resulting plasmid were named pJC1-PF1-U1A-TF1 (SEQ ID NO: 76) and pJC1-PF1-U1A-F30::broccoli/UUCG-TF1 (SEQ ID NO: 75), respectively.

PF1:
SEQ ID No: 67
CTCGAGCGGGACGGTCGAACCAGCTTCAAGCGACCGGATGAGTATGTTACAGTAGA
TAGCG
target RNA:
SEQ ID NO: 68
AGCGGGAGACCGCTCGACCTTAGTTCTCCTGTTGCGGGGGAGTTCATGGGATCCAG
GTACCCAGGGCGAGGCTTATCCATTGCACTCCGGATGTGCTGACCCCTG
F30 with broccoli in insertion site 1 and “TTCG” in insertion site 2:
SEQ ID NO: 69
TTGCCATGTGTATGTGGGAGACGGTCGGGTCCAGATATTCGTATCTGTCGAGTAGAG
TGTGGGCTCCCACATACTCTGATGATCCTTCGGGATCATTCATGGCAA
TF1:
SEQ ID NO: 70
CTAGCATAGCATAAAATAACGCCCCACCTTCTTAACGGGAGGTGGGGCGTTATTTTTA
CGGGGATCGCT
SEQ ID NO: 67-70 combined, with XbaI recognition site on 5′-end, SalI
recognition site on 3′-end:
SEQ ID NO: 71
CTGTCTCTAGACTCGAGCGGGACGGTCGAACCAGCTTCAAGCGACCGGATGAGTAT
GTTACAGTAGATAGCGAGCGGGAGACCGCTCGACCTTAGTTCTCCTGTTGCGGGGG
AGTTCATGGGATCCAGGTACCCAGGGCGAGGCTTATCCATTGCACTCCGGATGTGCT
GACCCCTGTTGCCATGTGTATGTGGGAGACGGTCGGGTCCAGATATTCGTATCTGTC
GAGTAGAGTGTGGGCTCCCACATACTCTGATGATCCTTCGGGATCATTCATGGCAAG
CTAGCATAGCATAAAATAACGCCCCACCTTCTTAACGGGAGGTGGGGCGTTATTTTTA
CGGTCGACCTGTC
SEQ ID NO: 67, 68 and 70, with XbaI recognition site on 5′-end, SalI
recognition site on 3′- end:
SEQ ID NO: 72
CTGTCTCTAGACTCGAGCGGGACGGTCGAACCAGCTTCAAGCGACCGGATGAGTAT
GTTACAGTAGATAGCGAGCGGGAGACCGCTCGACCTTAGTTCTCCTGTTGCGGGGG
AGTTCATGGGATCCAGGTACCCAGGGCGAGGCTTATCCATTGCACTCCGGATGTGCT
GACCCCTGGCTAGCATAGCATAAAATAACGCCCCACCTTCTTAACGGGAGGTGGGG
CGTTATTTTTACGGTCGACCTGTC
pJC1_check_f:
SEQ ID NO: 73
TGAAGACCGTCAACCAAAGG
pJC1_check_rev:
SEQ ID NO: 74
TGCCGGGAAGCTAGAGTAAG
pJC1-PF1-U1A-F30::broccoli/UUCG-TF1
SEQ ID NO: 75
CCGAGAAGTTTTTTACAAAAGGCAAAAACTTTTTCGGGATCGACAGAAATAAAACGAT
CGACGGTACGCAACAAAAAAGCGTCAGGATCGCCGTAGAGCGATTGAAGACCGTCA
ACCAAAGGGGAAGCCTCCAATCGACGCGACGCGCGCTCTACGGCGATCCTGACGCA
GATTTTTAGCTATCTGTCGCAGCGCCCTCAGGGACAAGCCACCCGCACAACGTCGC
GAGGGCGATCAGCGACGCCGCAGGGGGATCCTCTAGACTCGAGCGGGACGGTCGA
ACCAGCTTCAAGCGACCGGATGAGTATGTTACAGTAGATAGCGAGCGGGAGACCGC
TCGACCTTAGTTCTCCTGTTGCGGGGGAGTTCATGGGATCCAGGTACCCAGGGCGA
GGCTTATCCATTGCACTCCGGATGTGCTGACCCCTGTTGCCATGTGTATGTGGGAGA
CGGTCGGGTCCAGATATTCGTATCTGTCGAGTAGAGTGTGGGCTCCCACATACTCTG
ATGATCCTTCGGGATCATTCATGGCAAGCTAGCATAGCATAAAATAACGCCCCACCTT
CTTAACGGGAGGTGGGGCGTTATTTTTACGGTCGACCTGCAGCAATGGCAACAACGT
TGCGCAAACTATTAACTGGCGAACTACTTACTCTAGCTTCCCGGCAACAATTAATAGA
CTGGATGGAGGCGGATAAAGTTGCAGGACCACTTCTGCGCTCGGCCCTTCCGGCTG
GCTGGTTTATTGCTGATAAATCTGGAGCCGGTGAGCGTGGGTCTCGCGGTATCATTG
CAGCACTGGGGCCAGATGGTAAGCCCTCCCGTATCGTAGTTATCTACACGACGGGG
AGTCAGGCAACTATGGATGAACGAAATAGACAGATCGCTGAGATAGGTGCCTCACTG
ATTAAGCATTGGTAACTGTCAGACCAAGTTTACTCATATATACTTTAGATTGATTTAAA
ACTTCATTTTTAATTTAAAAGGATCTAGGTGAAGATCCTTTTTGATAATCTCATGACCA
AAATCCCTTAACGTGAGTTTTCGTTCCACTGAGCGTCAGACCCCTTAATAAGATGATC
TTCTTGAGATCGTTTTGGTCTGCGCGTAATCTCTTGCTCTGAAAACGAAAAAACCGCC
TTGCAGGGCGGTTTTTCGAAGGTTCTCTGAGCTACCAACTCTTTGAACCGAGGTAAC
TGGCTTGGAGGAGCGCAGTCACCAAAACTTGTCCTTTCAGTTTAGCCTTAACCGGCG
CATGACTTCAAGACTAACTCCTCTAAATCAATTACCAGTGGCTGCTGCCAGTGGTGCT
TTTGCATGTCTTTCCGGGTTGGACTCAAGACGATAGTTACCGGATAAGGCGCAGCGG
TCGGACTGAACGGGGGGTTCGTGCATACAGTCCAGCTTGGAGCGAACTGCCTACCC
GGAACTGAGTGTCAGGCGTGGAATGAGACAAACGCGGCCATAACAGCGGAATGACA
CCGGTAAACCGAAAGGCAGGAACAGGAGAGCGCACGAGGGAGCCGCCAGGGGGAA
ACGCCTGGTATCTTTATAGTCCTGTCGGGTTTCGCCACCACTGATTTGAGCGTCAGA
TTTCGTGATGCTTGTCAGGGGGGCGGAGCCTATGGAAAAACGGCTTTGCCGCGGCC
CTCTCACTTCCCTGTTAAGTATCTTCCTGGCATCTTCCAGGAAATCTCCGCCCCGTTC
GTAAGCCATTTCCGCTCGCCGCAGTCGAACGACCGAGCGTAGCGAGTCAGTGAGCG
AGGAAGCGGAATATATCCTGTATCACATATTCTGCTGACGCACCGGTGCAGCCTTTTT
TCTCCTGCCACATGAAGCACTTCACTGACACCCTCATCAGTGCCAACATAGTAAGCC
AGTATACACTCCGCTAGCGCTGAGGTCTGCCTCGTGAAGAAGGTGTTGCTGACTCAT
ACCAGGCCTGAATCGCCCCATCATCCAGCCAGAAAGTGAGGGAGCCACGGTTGATG
AGAGCTTTGTTGTAGGTGGACCAGTTGGTGATTTTGAACTTTTGCTTTGCCACGGAAC
GGTCTGCGTTGTCGGGAAGATGCGTGATCTGATCCTTCAACTCAGCAAAAGTTCGAT
TTATTCAACAAAGCCACGTTGTGTCTCAAAATCTCTGATGTTACATTGCACAAGATAAA
AATATATCATCATGAACAATAAAACTGTCTGCTTACATAAACAGTAATACAAGGGGTGT
TATGAGCCATATTCAACGGGAAACGTCTTGCTCGAGGCCGCGATTAAATTCCAACAT
GGATGCTGATTTATATGGGTATAAATGGGCTCGCGATAATGTCGGGCAATCAGGTGC
GACAATCTATCGATTGTATGGGAAGCCCGATGCGCCAGAGTTGTTTCTGAAACATGG
CAAAGGTAGCGTTGCCAATGATGTTACAGATGAGATGGTCAGACTAAACTGGCTGAC
GGAATTTATGCCTCTTCCGACCATCAAGCATTTTATCC
GTACTCCTGATGATGCATGGTTACTCACCACTGCGATCCCCGGGAAAACA
GCATTCCAGGTATTAGAAGAATATCCTGATTCAGGTGAAAATATTGTTGA
TGCGCTGGCAGTGTTCCTGCGCCGGTTGCATTCGATTCCTGTTTGTAATT
GTCCTTTTAACAGCGATCGCGTATTTCGTCTCGCTCAGGCGCAATCACGA
ATGAATAACGGTTTGGTTGATGCGAGTGATTTTGATGACGAGCGTAATGG
CTGGCCTGTTGAACAAGTCTGGAAAGAAATGCATAAGCTTTTGCCATTCT
CACCGGATTCAGTCGTCACTCATGGTGATTTCTCACTTGATAACCTTATT
TTTGACGAGGGGAAATTAATAGGTTGTATTGATGTTGGACGAGTCGGAAT
CGCAGACCGATACCAGGATCTTGCCATCCTATGGAACTGCCTCGGTGAGT
TTTCTCCTTCATTACAGAAACGGCTTTTTCAAAAATATGGTATTGATAAT
CCTGATATGAATAAATTGCAGTTTCATTTGATGCTCGATGAGTTTTTCTA
ATCAGAATTGGTTAATTGGTTGTAACACTGGCAGAGCATTACGCTGACTT
GACGGGACGGCGGCTTTGTTGAATAAATCGAACTTTTGCTGAGTTGAAGG
ATCAGATCACGCATCTTCCCGACAACGCAGACCGTTCCGTGGCAAAGCAA
AAGTTCAAAATCACCAACTGGTCCACCTACAACAAAGCTCTCATCAACCG
TGGCTCCCTCACTTTCTGGCTGGATGATGGGGCGATTCAGGCCTGGTATG
AGTCAGCAACACCTTCTTCACGAGGCAGACCTCAGCGCTCAAAGATGCAG
GGGTAAAAGCTAACCGCATCTTTACCGACAAGGCATCCGGCAGTTCAACA
GATCGGGAAGGGCTGGATTTGCTGAGGATGAAGGTGGAGGAAGGTGATGT
CATTCTGGTGAAGAAGCTCGACCGTCTTGGCCGCGACACCGCCGACATGA
TCCAACTGATAAAAGAGTTTGATGCTCAGGGTGTAGCGGTTCGGTTTATT
GACGACGGGATCAGTACCGACGGTGATATGGGGCAAATGGTGGTCACCAT
CCTGTCGGCTGTGGCACAGGCTGAACGCCGGAGGATCAAGTCGGTCAAGC
CAAGCGCAACCAGCGGCACCGCCGCGAGCAACGTCGCAAGGGCGATCAGG
GGACGATTTTTGCGAAGAATTTCCACGGTAAGAATCCAATCTCTCGAATT
TAGGGTGAAAGAAGCTTGGCATAGGGGTGTGCACGAACTCGGTGGAGGAA
ATTTCCGCGGGGCAAGGCTTCGCGAAGCGGAGTCGCGGCAGTGGCTTTGA
AGATCTTTGGGAGCAGTCCTTGTGCGCTTACGAGGTGAGCCGGTGGGGAA
CCGTTATCTGCCTATGGTGTGAGCCCCCCTAGAGAGCTTCAAGAGCAATC
AGCCCGACCTAGAAAGGAGGCCAAGAGAGAGACCCCTACGGGGGGAACCG
TTTTCTGCCTACGAGATGGCACATTTACTGGGAAGCTTTACGGCGTCCTC
GTGGAAGTTCAATGCCCGCAGACTTAAGTGCTCTATTCACGGTCTGACGT
GACACGCTAAATTCAGACATAGCTTCATTGATTGTCGGCCACGAGCCAGT
CTCTCCCTCAACAGTCATAAACCAACCTGCAATGGTCAAGCGATTTCCTT
TAGCTTTCCTAGCTTGTCGTTGACTGGACTTAGCTAGTTTTTCTCGCTGT
GCTCGGGCGTACTCACTGTTTGGGTCTTTCCAGCGTTCTGCGGCCTTTTT
ACCGCCACGTCTTCCCATAGTGGCCAGAGCTTTTCGCCCTCGGCTGCTCT
GCGTCTCTGTCTGACGAGCAGGGACGACTGGCTGGCCTTTAGCGACGTAG
CCGCGCACACGTCGCGCCATCGTCTGGCGGTCACGCATCGGCGGCAGATC
AGGCTCACGGCCGTCTGCTCCGACCGCCTGAGCGACGGTGTAGGCACGCT
CGTAGGCGTCGATGATCTTGGTGTCTTTTAGGCGCTCACCAGCCGCTTTT
AACTGGTATCCCACAGTCAAAGCGTGGCGAAAAGCCGTCTCATCACGGGC
GGCACGCCCTGGAGCAGTCCAGAGGACACGGACGCCGTCGATCAGCTCTC
CAGACGCTTCAGCGGCGCTCGGCAGGCTTGCTTCAAGCGTGGCAAGTGCT
TTTGCTTCCGCAGTGGCTTTTCTTGCCGCTTCGATACGTGCCCGTCCGCT
AGAAAACTCCTGCTCATAGCGTTTTTTAGGTTTTTCTGTGCCTGAGATCA
TGCGAGCAACCTCCATAAGATCAGCTAGGCGATCCACGCGATTGTGCTGG
GCATGCCAGCGGTACGCGGTGGGATCGTCGGAGACGTGCAGTGGCCACCG
GCTCAGCCTATGTGAAAAAGCCTGGTCAGCGCCGAAAACGCGGGTCATTT
CCTCGGTCGTTGCAGCCAGCAGGCGCATATTCGGGCTGCTCATGCCTGCT
GCGGCATACACCGGATCAATGAGCCAGATGAGCTGGCATTTCCCGCTCAG
TGGATTCACGCCGATCCAAGCTGGCGCTTTTTCCAGGCGTGCCCAGCGCT
CCAAAATCGCGTAGACCTCGGGGTTTACGTGCTCGATTTTCCCGCCGGCC
TGGTGGCTCGGCACATCAATGTCCAGGACAAGCACGGCTGCGTGCTGCGC
GTGCGTCAGAGCAACATACTGGCACCGGGCAAGCGATTTTGAACCAACTC
GGTATAACTTCGGCTGTGTTTCTCCCGTGTCCGGGTCTTTGATCCAAGCG
CTGGCGAAGTCGCGGGTCTTGCTGCCCTGGAAATTTTCTCTGCCCAGGTG
AGCGAGGAATTCGCGGCGGTCTTCGCTCGTCCAGCCACGTGATCGCAGCG
CGAGCTCGGGATGGGTGTCGAACAGATCAGCGGAAAATTTCCAGGCCGGT
GTGTCAATGTCTCGTGAATCCGCTAGAGTCATTTTTGAGCGCTTTCTCCC
AGGTTTGGACTGGGGGTTAGCCGACGCCCTGTGAGTTACCGCTCACGGGG
CGTTCAACATTTTTCAGGTATTCGTGCAGCTTATCGCTTCTTGCCGCCTG
TGCGCTTTTTCGACGCGCGACGCTGCTGCCGATTCGGTGCAGGTGGTGGC
GGCGCTGACACGTCCTGGGCGGCCACGGCCACACGAAACGCGGCATTTAC
GATGTTTGTCATGCCTGCGGGCACCGCGCCACGATCGCGGATAATTCTCG
CTGCCGCTTCCAGCTCTGTGACGACCATGGCCAAAATTTCGCTCGGGGGA
CGCACTTCCAGCGCCATTTGCGACCTAGCCGCCTCCAGCTCCTCGGCGTG
GCGTTTGTTGGCGCGCTCGCGGCTGGCTGCGGCACGACACGCATCTGAGC
AATATTTTGCGCGCCGTCCTCGCGGGTCAGGCCGGGGAGGAATCAGGCCA
CCGCAGTAGGCGCAACTGATTCGATCCTCCACTACTGTGCGTCCTCCTGG
CGCTGCCGAGCACGCAGCTCGTCAGCCAGCTCCTCAAGATCCGCCACGAG
AGTTTCTAGGTCGCTCGCGGCACTGGCCCAGTCTCGTGATGCTGGCGCGT
CCGTCGTATCGAGAGCTCGGAAAAATCCGATCACCGTTTTTAAATCGACG
GCAGCATCGAGCGCGTCGGACTCCAGCGCGACATCAGAGAGATCCATAGC
TGATGATTCGGGCCAATTTTGGTACTTCGTCGTGAAGGTCATGACACCAT
TATAACGAACGTTCGTTAAAGTTTTTGGCGGAAAATCACGCGGCACGAAA
ATTTTCACGAAGCGGGACTTTGCGCAGCTCAGGGGTGCTAAAAATTTTGT
ATCGCACTTGATTTTTCCGAAAGACAGATTATCTGCAAACGGTGTGTCGT
ATTTCTGGCTTGGTTTTTAAAAAATCTGGAATCGAAAATTTGCGGGGCGA
pJC1-PF1-U1A-TF1:
SEQ ID NO: 76
CCGAGAAGTTTTTTACAAAAGGCAAAAACTTTTTCGGGATCGACAGAAAT
AAAACGATCGACGGTACGCAACAAAAAAGCGTCAGGATCGCCGTAGAGCG
ATTGAAGACCGTCAACCAAAGGGGAAGCCTCCAATCGACGCGACGCGCGC
TCTACGGCGATCCTGACGCAGATTTTTAGCTATCTGTCGCAGCGCCCTCA
GGGACAAGCCACCCGCACAACGTCGCGAGGGCGATCAGCGACGCCGCAGG
GGGATCCTCTAGACTCGAGCGGGACGGTCGAACCAGCTTCAAGCGACCGG
ATGAGTATGTTACAGTAGATAGCGAGCGGGAGACCGCTCGACCTTAGTTC
TCCTGTTGCGGGGGAGTTCATGGGATCCAGGTACCCAGGGCGAGGCTTAT
CCATTGCACTCCGGATGTGCTGACCCCTGGCTAGCATAGCATAAAATAAC
GCCCCACCTTCTTAACGGGAGGTGGGGCGTTATTTTTACGGGGATCGCTT
CTTCCGCTTCCTCGCTCACTGACTCGCTGCGCTCGGTCGTTCGGCTGCGG
GTCGACCTGCAGCAATGGCAACAACGTTGCGCAAACTATTAACTGGCGAA
CTACTTACTCTAGCTTCCCGGCAACAATTAATAGACTGGATGGAGGCGGA
TAAAGTTGCAGGACCACTTCTGCGCTCGGCCCTTCCGGCTGGCTGGTTTA
TTGCTGATAAATCTGGAGCCGGTGAGCGTGGGTCTCGCGGTATCATTGCA
GCACTGGGGCCAGATGGTAAGCCCTCCCGTATCGTAGTTATCTACACGAC
GGGGAGTCAGGCAACTATGGATGAACGAAATAGACAGATCGCTGAGATAG
GTGCCTCACTGATTAAGCATTGGTAACTGTCAGACCAAGTTTACTCATAT
ATACTTTAGATTGATTTAAAACTTCATTTTTAATTTAAAAGGATCTAGGT
GAAGATCCTTTTTGATAATCTCATGACCAAAATCCCTTAACGTGAGTTTT
CGTTCCACTGAGCGTCAGACCCCTTAATAAGATGATCTTCTTGAGATCGT
TTTGGTCTGCGCGTAATCTCTTGCTCTGAAAACGAAAAAACCGCCTTGCA
GGGCGGTTTTTCGAAGGTTCTCTGAGCTACCAACTCTTTGAACCGAGGTA
ACTGGCTTGGAGGAGCGCAGTCACCAAAACTTGTCCTTTCAGTTTAGCCT
TAACCGGCGCATGACTTCAAGACTAACTCCTCTAAATCAATTACCAGTGG
CTGCTGCCAGTGGTGCTTTTGCATGTCTTTCCGGGTTGGACTCAAGACGA
TAGTTACCGGATAAGGCGCAGCGGTCGGACTGAACGGGGGGTTCGTGCAT
ACAGTCCAGCTTGGAGCGAACTGCCTACCCGGAACTGAGTGTCAGGCGTG
GAATGAGACAAACGCGGCCATAACAGCGGAATGACACCGGTAAACCGAAA
GGCAGGAACAGGAGAGCGCACGAGGGAGCCGCCAGGGGGAAACGCCTGGT
ATCTTTATAGTCCTGTCGGGTTTCGCCACCACTGATTTGAGCGTCAGATT
TCGTGATGCTTGTCAGGGGGGCGGAGCCTATGGAAAAACGGCTTTGCCGC
GGCCCTCTCACTTCCCTGTTAAGTATCTTCCTGGCATCTTCCAGGAAATC
TCCGCCCCGTTCGTAAGCCATTTCCGCTCGCCGCAGTCGAACGACCGAGC
GTAGCGAGTCAGTGAGCGAGGAAGCGGAATATATCCTGTATCACATATTC
TGCTGACGCACCGGTGCAGCCTTTTTTCTCCTGCCACATGAAGCACTTCA
CTGACACCCTCATCAGTGCCAACATAGTAAGCCAGTATACACTCCGCTAG
CGCTGAGGTCTGCCTCGTGAAGAAGGTGTTGCTGACTCATACCAGGCCTG
AATCGCCCCATCATCCAGCCAGAAAGTGAGGGAGCCACGGTTGATGAGAG
CTTTGTTGTAGGTGGACCAGTTGGTGATTTTGAACTTTTGCTTTGCCACG
GAACGGTCTGCGTTGTCGGGAAGATGCGTGATCTGATCCTTCAACTCAGC
AAAAGTTCGATTTATTCAACAAAGCCACGTTGTGTCTCAAAATCTCTGAT
GTTACATTGCACAAGATAAAAATATATCATCATGAACAATAAAACTGTCT
GCTTACATAAACAGTAATACAAGGGGTGTTATGAGCCATATTCAACGGGA
AACGTCTTGCTCGAGGCCGCGATTAAATTCCAACATGGATGCTGATTTAT
ATGGGTATAAATGGGCTCGCGATAATGTCGGGCAATCAGGTGCGACAATC
TATCGATTGTATGGGAAGCCCGATGCGCCAGAGTTGTTTCTGAAACATGG
CAAAGGTAGCGTTGCCAATGATGTTACAGATGAGATGGTCAGACTAAACT
GGCTGACGGAATTTATGCCTCTTCCGACCATCAAGCATTTTATCCGTACT
CCTGATGATGCATGGTTACTCACCACTGCGATCCCCGGGAAAACAGCATT
CCAGGTATTAGAAGAATATCCTGATTCAGGTGAAAATATTGTTGATGCGC
TGGCAGTGTTCCTGCGCCGGTTGCATTCGATTCCTGTTTGTAATTGTCCT
TTTAACAGCGATCGCGTATTTCGTCTCGCTCAGGCGCAATCACGAATGAA
TAACGGTTTGGTTGATGCGAGTGATTTTGATGACGAGCGTAATGGCTGGC
CTGTTGAACAAGTCTGGAAAGAAATGCATAAGCTTTTGCCATTCTCACCG
GATTCAGTCGTCACTCATGGTGATTTCTCACTTGATAACCTTATTTTTGA
CGAGGGGAAATTAATAGGTTGTATTGATGTTGGACGAGTCGGAATCGCAG
ACCGATACCAGGATCTTGCCATCCTATGGAACTGCCTCGGTGAGTTTTCT
CCTTCATTACAGAAACGGCTTTTTCAAAAATATGGTATTGATAATCCTGA
TATGAATAAATTGCAGTTTCATTTGATGCTCGATGAGTTTTTCTAATCAG
AATTGGTTAATTGGTTGTAACACTGGCAGAGCATTACGCTGACTTGACGG
GACGGCGGCTTTGTTGAATAAATCGAACTTTTGCTGAGTTGAAGGATCAG
ATCACGCATCTTCCCGACAACGCAGACCGTTCCGTGGCAAAGCAAAAGTT
CAAAATCACCAACTGGTCCACCTACAACAAAGCTCTCATCAACCGTGGCT
CCCTCACTTTCTGGCTGGATGATGGGGCGATTCAGGCCTGGTATGAGTCA
GCAACACCTTCTTCACGAGGCAGACCTCAGCGCTCAAAGATGCAGGGGTA
AAAGCTAACCGCATCTTTACCGACAAGGCATCCGGCAGTTCAACAGATCG
GGAAGGGCTGGATTTGCTGAGGATGAAGGTGGAGGAAGGTGATGTCATTC
TGGTGAAGAAGCTCGACCGTCTTGGCCGCGACACCGCCGACATGATCCAA
CTGATAAAAGAGTTTGATGCTCAGGGTGTAGCGGTTCGGTTTATTGACGA
CGGGATCAGTACCGACGGTGATATGGGGCAAATGGTGGTCACCATCCTGT
CGGCTGTGGCACAGGCTGAACGCCGGAGGATCAAGTCGGTCAAGCCAAGC
GCAACCAGCGGCACCGCCGCGAGCAACGTCGCAAGGGCGATCAGGGGACG
ATTTTTGCGAAGAATTTCCACGGTAAGAATCCAATCTCTCGAATTTAGGG
TGAAAGAAGCTTGGCATAGGGGTGTGCACGAACTCGGTGGAGGAAATTTC
CGCGGGGCAAGGCTTCGCGAAGCGGAGTCGCGGCAGTGGCTTTGAAGATC
TTTGGGAGCAGTCCTTGTGCGCTTACGAGGTGAGCCGGTGGGGAACCGTT
ATCTGCCTATGGTGTGAGCCCCCCTAGAGAGCTTCAAGAGCAATCAGCCC
GACCTAGAAAGGAGGCCAAGAGAGAGACCCCTACGGGGGGAACCGTTTTC
TGCCTACGAGATGGCACATTTACTGGGAAGCTTTACGGCGTCCTCGTGGA
AGTTCAATGCCCGCAGACTTAAGTGCTCTATTCACGGTCTGACGTGACAC
GCTAAATTCAGACATAGCTTCATTGATTGTCGGCCACGAGCCAGTCTCTC
CCTCAACAGTCATAAACCAACCTGCAATGGTCAAGCGATTTCCTTTAGCT
TTCCTAGCTTGTCGTTGACTGGACTTAGCTAGTTTTTCTCGCTGTGCTCG
GGCGTACTCACTGTTTGGGTCTTTCCAGCGTTCTGCGGCCTTTTTACCGC
CACGTCTTCCCATAGTGGCCAGAGCTTTTCGCCCTCGGCTGCTCTGCGTC
TCTGTCTGACGAGCAGGGACGACTGGCTGGCCTTTAGCGACGTAGCCGCG
CACACGTCGCGCCATCGTCTGGCGGTCACGCATCGGCGGCAGATCAGGCT
CACGGCCGTCTGCTCCGACCGCCTGAGCGACGGTGTAGGCACGCTCGTAG
GCGTCGATGATCTTGGTGTCTTTTAGGCGCTCACCAGCCGCTTTTAACTG
GTATCCCACAGTCAAAGCGTGGCGAAAAGCCGTCTCATCACGGGGGCAC
GCCCTGGAGCAGTCCAGAGGACACGGACGCCGTCGATCAGCTCTCCAGAC
GCTTCAGCGGCGCTCGGCAGGCTTGCTTCAAGCGTGGCAAGTGCTTTTGC
TTCCGCAGTGGCTTTTCTTGCCGCTTCGATACGTGCCCGTCCGCTAGAAA
ACTCCTGCTCATAGCGTTTTTTAGGTTTTTCTGTGCCTGAGATCATGCGA
GCAACCTCCATAAGATCAGCTAGGCGATCCACGCGATTGTGCTGGGCATG
CCAGCGGTACGCGGTGGGATCGTCGGAGACGTGCAGTGGCCACCGGCTCA
GCCTATGTGAAAAAGCCTGGTCAGCGCCGAAAACGCGGGTCATTTCCTCG
GTCGTTGCAGCCAGCAGGCGCATATTCGGGCTGCTCATGCCTGCTGCGGC
ATACACCGGATCAATGAGCCAGATGAGCTGGCATTTCCCGCTCAGTGGAT
TCACGCCGATCCAAGCTGGCGCTTTTTCCAGGCGTGCCCAGCGCTCCAAA
ATCGCGTAGACCTCGGGGTTTACGTGCTCGATTTTCCCGCCGGCCTGGTG
GCTCGGCACATCAATGTCCAGGACAAGCACGGCTGCGTGCTGCGCGTGCG
TCAGAGCAACATACTGGCACCGGGCAAGCGATTTTGAACCAACTCGGTAT
AACTTCGGCTGTGTTTCTCCCGTGTCCGGGTCTTTGATCCAAGCGCTGGC
GAAGTCGCGGGTCTTGCTGCCCTGGAAATTTTCTCTGCCCAGGTGAGCGA
GGAATTCGCGGCGGTCTTCGCTCGTCCAGCCACGTGATCGCAGCGCGAGC
TCGGGATGGGTGTCGAACAGATCAGCGGAAAATTTCCAGGCCGGTGTGTC
AATGTCTCGTGAATCCGCTAGAGTCATTTTTGAGCGCTTTCTCCCAGGTT
TGGACTGGGGGTTAGCCGACGCCCTGTGAGTTACCGCTCACGGGGCGTTC
AACATTTTTCAGGTATTCGTGCAGCTTATCGCTTCTTGCCGCCTGTGCGC
TTTTTCGACGCGCGACGCTGCTGCCGATTCGGTGCAGGTGGTGGCGGCGC
TGACACGTCCTGGGCGGCCACGGCCACACGAAACGCGGCATTTACGATGT
TTGTCATGCCTGCGGGCACCGCGCCACGATCGCGGATAATTCTCGCTGCC
GCTTCCAGCTCTGTGACGACCATGGCCAAAATTTCGCTCGGGGGACGCAC
TTCCAGCGCCATTTGCGACCTAGCCGCCTCCAGCTCCTCGGCGTGGCGTT
TGTTGGCGCGCTCGCGGCTGGCTGCGGCACGACACGCATCTGAGCAATAT
TTTGCGCGCCGTCCTCGCGGGTCAGGCCGGGGAGGAATCAGGCCACCGCA
GTAGGCGCAACTGATTCGATCCTCCACTACTGTGCGTCCTCCTGGCGCTG
CCGAGCACGCAGCTCGTCAGCCAGCTCCTCAAGATCCGCCACGAGAGTTT
CTAGGTCGCTCGCGGCACTGGCCCAGTCTCGTGATGCTGGCGCGTCCGTC
GTATCGAGAGCTCGGAAAAATCCGATCACCGTTTTTAAATCGACGGCAGC
ATCGAGCGCGTCGGACTCCAGCGCGACATCAGAGAGATCCATAGCTGATG
ATTCGGGCCAATTTTGGTACTTCGTCGTGAAGGTCATGACACCATTATAA
CGAACGTTCGTTAAAGTTTTTGGCGGAAAATCACGCGGCACGAAAATTTT
CACGAAGCGGGACTTTGCGCAGCTCAGGGGTGCTAAAAATTTTGTATCGC
ACTTGATTTTTCCGAAAGACAGATTATCTGCAAACGGTGTGTCGTATTTC
TGGCTTGGTTTTTAAAAAATCTGGAATCGAAAATTTGCGGGGCGA

b) Transformation of Corynebacterium glutamicum with pJC1-PF1-U1A-F30::broccoli/UUCG-TF1 and pJC1-PF1-U1A-TF1

Competent cells of the C. glutamicum strain ATCC 13032 Δcg2273 were prepared and transformed with pJC1 PF1 U1A F30::broccoli/UUCG-TF1 and pJC1-PF1-U1A-TF1 according to Tauch et al., 2002 (Tauch, A., Kirchner, O., Loffler, B., Götker, S., Pühler, A., and Kalinowski, J. Efficient Electrotransformation of Corynebacterium diphtheriae with a Mini-Replicon Derived from the Corynebacterium glutamicum Plasmid pGA1. Curr. Microbiol. 2002; 45, 362-367). The selection of the transformants was carried out on CGIII (Menkel, E., Thierbach, G., Eggeling, L., and Sahm, H. Influence of increased aspartate availability on lysine formation by a recombinant strain of Corynebacterium glutamicum and utilization of fumarate. Appl. Environ. Microbiol. 1989; 55, 684-688) agar (1%) plates with 15 μg/ml of kanamycin. Clones thus obtained were named C. glutamicum ATCC 13032 Δcg2273 pJC1-PF1-U1A-F30::broccoli/UUCG-TF1 or C. glutamicum ATCC 13032 Δcg2273 pJC1-PF1-U1A-TF1, depending on which plasmid was used for transformation.

c) Mutagenesis of C. glutamicum ATCC 13032Δcg2273 pJC1-PF1-U1A-F30::broccoli/UUCG-TF1

The produced strain C. glutamicum ATCC 13032 Δcg2273 pJC1-PF1-U1A-F30::broccoli/UUCG-TF1 was cultured overnight in CGIII medium at 30° C., 120 rpm, 10 mL total volume with 15 μg/mL kanamycin added to the medium. Cells from this preculture were used to prepare a cell suspension with an OD₆₀₀ of 0.5 in 5 mL total volume of phosphate-buffered-saline (PBS). N-Methyl-N′-nitro-N-nitrosoguanidine (MNNG) was added to a final concentration of 25 μg/mL. After 20 min of incubation, a 1.5 mL sample was taken, centrifuged (2000 rpm, 2 min) and resuspended in 2 mL PBS. This washing step was repeated twice prior to final resuspension in 1.5 mL PBS and transfer into 15 mL fresh CGIII cultivation medium with 15 μg/mL kanamycin. The culture was subjected to a 16-hour cultivation at 30° C., 120 rpm in a non-baffled shake flask.

d) Identification and Isolation of Cells that Show the Highest Intensity of Fluorescence and Therefore a High Expression of the RNA of Interest by the Means of Fluorescence-Activated Cell Sorting (FACS)

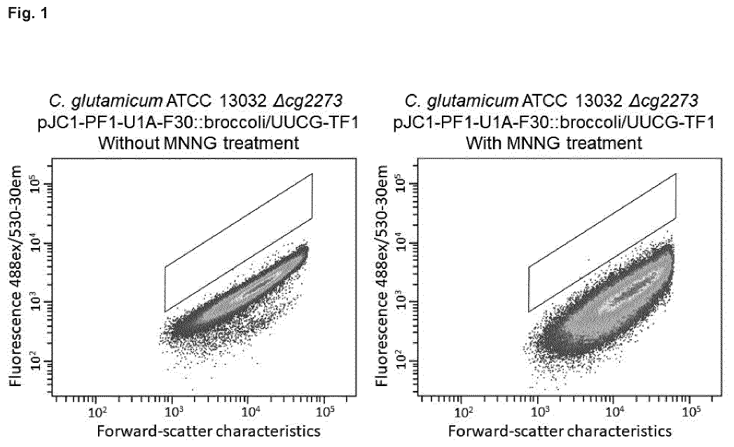

The regeneration culture from c) was diluted to an OD of 0.6 using PBS with a final concentration of 500 μM DFHBI. After 10 min of incubation, the cell suspension was analyzed in an AriaIII High-speed cell sorter (BD Biosciences, Franklin Lakes, NJ, USA) equipped with a 70 μm nozzle and run with a sheath pressure of 70 psi. A 488 nm blue solid laser was used for excitation. Forward-scatter characteristics (FSC) were recorded as small-angle scatter and side-scatter characteristics (SSC) were recorded as orthogonal scatter of the 488 nm laser. A 502 nm long-pass and 530/30 nm band-pass filter combination were used for fluorescence detection. FACSDiva 8.0.1 (BD Biosciences, San Jose, USA) was used for FACS control and data analysis. Prior to data acquisition, debris and electronic noise were excluded from the analysis by electronic gating in the FSC-H against SSC-H plot. Another gating step was performed on the resulting population in the FSC-H against FSC-W plot to exclude doublets. Fluorescence acquisition was performed with the population resulting from this two-step gating (FIG. 1). 96 cells that exhibited an increased fluorescence in comparison to C. glutamicum ATCC 13032 Δcg2273 pJC1-PF1-U1A-F30::broccoli/UUCG-TF1 without MNNG treatment were isolated from the culture broth and placed on CGIII agar plates (1%) with 15 μg/mL kanamycin.

### e) Cultivation of Cells Isolated in d) for Phenotype Validation

Of the isolated cells, 19 grew into colonies within the next 48 h of incubation at 30° C. and were used to inoculate 10 mL of CGIII medium. Cultivation of the isolated cells took place at 30° C., 120 rpm in a non-baffled shake flask. After 20 h of cultivation, the culture broths were diluted to an OD of 0.6 using PBS with a final concentration of 500 μM DFHBI. After 10 min of incubation, the cell suspension was analyzed in an AriaIII High-speed cell sorter using the settings listed in d). Of the 19 cultures cultivated, 4 showed an 1.5- to 2-fold increased fluorescence in comparison to the starting strain C. glutamicum ATCC 13032 Δcg2273 pJC1-PF1-U1A-F30::broccoli/UUCG-TF1.

f) Extraction of the RNA of Interest from the Cells Isolated in d)

Using the culture broths analyzed in e), 1×10⁹ cells from the four best performing strains were used for RNA extraction with the Monarch total RNA kit (New England Biolabs, Ipswich, MA, USA). The isolated RNA was analyzed using an Agilent Bioanalyzer 2100 (Agilent Technologies, Santa Clara, CA, USA). The increased fluorescence corresponded to an increased target RNA abundance per total RNA extracted.

TABLE 1

| | Specific fluorescence relative |
|---|---|
| | to the control strain/target |
| | RNA titer relative to the |
| Strain | control strain |
| C. glutamicum ATCC 13032 Δcg2273 | 100%/100% |
| pJC1-PF1-U1A-F30::broccoli/UUCG-TF1 | |
| (Control) | |
| Mutant 1 | 227%/220% |
| Mutant 2 | 181%/185% |
| Mutant 3 | 171%/174% |
| Mutant 4 | 152%/149% |

g) Using Isolated Strains to Produce Target RNA without Tag

The plasmid pJC1-PF1-U1A-F30::broccoli/UUCG-TF1 was removed from the isolated strains using an adapted version of the transformation protocol of Tauch et al., 2002 (Tauch, A., Kirchner, O., Löffler, B., Götker, S., Pühler, A., and Kalinowski, J. Efficient Electrotransformation of Corynebacterium diphtheriae with a Mini-Replicon Derived from the Corynebacterium glutamicum Plasmid pGA1. Curr. Microbiol. 2002; 45, 362-367). Strains were made competent according to Tauch et al., 2002 and electroporation was performed without addition of plasmid DNA. Following the regeneration according to the original protocol, the cells were diluted and spread on non-selective CGIII agar. Grown colonies were streaked on CGIII agar with 15 μg/mL kanamycin and non-selective CGIII agar. Successful removal of pJC1-PF1-U1A-F30::broccoli/UUCG-TF1 from kanamycin-sensitive cells was confirmed by colony PCR (no product with primer combination pJC1_check_f and pJC1_check_rev). The plasmid-free strains thus produced were transformed with pJC1-PF1-U1A-TF1 as described in b) to enable production of the target RNA without the tag consisting of the F30 scaffold and broccoli. Cultivation of the strains according to the description in e) and extraction and analysis of the produced RNA as described in f) confirmed increased target RNA production in comparison to the control strain C. glutamicum ATCC 13032 Δcg2273 pJC1-PF1-U1A-TF1.

This experiment shows that the invention enables the isolation of cells with improved production of a heterologous RNA of interest from a mutagenized cell broth by linking a hitherto unsuspicious phenotype (RNA production) with a fluorescence output.

### Example 2

a) Construction of the Vector pK19msB_16S rRNA-broccoli

The construction of the plasmid was achieved by means of chemical synthesis of a synthetic DNA-fragment (SEQ ID NO: 79 for 16S rRNA-broccoli), and its insertion into restriction sites EcoRI and HindIII of pK19mobsacB resulting in plasmid pK19msB_16S rRNA-broccoli (SEQ ID NO: 80) (ordered from Twist Bioscience, South San Francisco, USA). SEQ ID NO: 79 contained 601 bp upstream of the aptamer integration site (SEQ ID NO: 81), a restriction site for verification of positive integration (SEQ ID NO:122: tctaga), the F30 scaffold with a broccoli aptamer in the insertion site (SEQ ID NO: 69) and 479 bp downstream of the target integration site (SEQ ID NO: 83).

SEQ ID NO: 81, 82, 83 and 69 combined:
SEQ ID NO: 79
CGCACAAGCGGCGGAGCATGTGGATTAATTCGATGCAACGCGAAG
AACCTTACCTGGGCTTGACATGGACCGGATCGGCGTAGAGATACG
TTTTCCCTTGTGGTCGGTTCACAGGTGGTGCATGGTTGTCGTCAG
CTCGTGTCGTGAGATGTTGGGTTAAGTCCCGCAACGAGCGCAACC
CTTGTCTTATGTTGCCAGCACATTGTGGTGGGTACTCATGAGAGA
CTGCCGGGGTTAACTCGGAGGAAGGTGGGGATGACGTCAAATCAT
CATGCCCCTTATGTCCAGGGCTTCACACATGCTACAATGGTCGGT
ACAGCGAGTTGCCACACCGTGAGGTGGAGCTAATCTCTTAAAGCC
GGCCTCAGTTCGGATTGGGGTCTGCAACTCGACCCCATGAAGTCG
GAGTCGCTAGTAATCGCAGATCAGCAACGCTGCGGTGAATACGTT
CCCGGGCCTTGTACACACCGCCCGTCACGTCATGAAAGTTGGTAA
CACCCGAAGCCAGTGGCCCAACCTTTTAGGGGGGAGCTGTCGAAG
GTGGGATCGGCGATTGGGACGAAGTCGTAACAAGGTAGCCGTACC
GGAAGGTGCGGCTGGATCTAGATTGCCATGTGTATGTGGGAGACG
GTCGGGTCCAGATATTCGTATCTGTCGAGTAGAGTGTGGGCTCCC
ACATACTCTGATGATCCTTCGGGATCATTCATGGCAACCTCCTTT
CTAAGGAGCTTTATTAACCCACATCAGACTGTGTCTGGTTGGTGG
GTTGTTGGTGTTGGAACCCGTATGTGGTTGCCATCAACATATTTT
TAATCGGGTGGAGATGACCCCTCGGGTGACAACAACACAGCAAAC
AGTGCTGTGATTAATAGGTGGCATGCTGTTGGGTGTCTGGAATGA
CATCGCAAGCATCACCTTTTGGTGGTGTGTGTGGGTTGTTTCTAA
CATCGAGCATCGTCAACACGGGTAGAGAATGTTGTGTTCTTTGGT
TGTGGTGGGGGTGGTGTGTTGTGTGAGAACTGTATAGTGGACGCG
AGCATCTTTATTTTTTTGTTTTTTGTTGTGTGATACCGAACGCGC
CCGCACTTTGTGTGTGGGTTATAGTATTTTGTTTGTTGTTTTGTA
GGGCACACGGTGGATGCCTTGGCATATCAAGCCGATGAAGGACGT
GAGAGGCTGCGTTATGCCTCG
pK19msB_16S rRNA-broccoli:
SEQ ID NO: 80
CGCACAAGCGGCGGAGCATGTGGATTAATTCGATGCAACGCGAAG
AACCTTACCTGGGCTTGACATGGACCGGATCGGCGTAGAGATACG
TTTTCCCTTGTGGTCGGTTCACAGGTGGTGCATGGTTGTCGTCAG
CTCGTGTCGTGAGATGTTGGGTTAAGTCCCGCAACGAGCGCAACC
CTTGTCTTATGTTGCCAGCACATTGTGGTGGGTACTCATGAGAGA
CTGCCGGGGTTAACTCGGAGGAAGGTGGGGATGACGTCAAATCAT
CATGCCCCTTATGTCCAGGGCTTCACACATGCTACAATGGTCGGT
ACAGCGAGTTGCCACACCGTGAGGTGGAGCTAATCTCTTAAAGCC
GGCCTCAGTTCGGATTGGGGTCTGCAACTCGACCCCATGAAGTCG
GAGTCGCTAGTAATCGCAGATCAGCAACGCTGCGGTGAATACGTT
CCCGGGCCTTGTACACACCGCCCGTCACGTCATGAAAGTTGGTAA
CACCCGAAGCCAGTGGCCCAACCTTTTAGGGGGGAGCTGTCGAAG
GTGGGATCGGCGATTGGGACGAAGTCGTAACAAGGTAGCCGTACC
GGAAGGTGCGGCTGGATCTAGATTGCCATGTGTATGTGGGAGACG
GTCGGGTCCAGATATTCGTATCTGTCGAGTAGAGTGTGGGCTCCC
ACATACTCTGATGATCCTTCGGGATCATTCATGGCAACCTCCTTT
CTAAGGAGCTTTATTAACCCACATCAGACTGTGTCTGGTTGGTGG
GTTGTTGGTGTTGGAACCCGTATGTGGTTGCCATCAACATATTTT
TAATCGGGTGGAGATGACCCCTCGGGTGACAACAACACAGCAAAC
AGTGCTGTGATTAATAGGTGGCATGCTGTTGGGTGTCTGGAATGA
CATCGCAAGCATCACCTTTTGGTGGTGTGTGTGGGTTGTTTCTAA
CATCGAGCATCGTCAACACGGGTAGAGAATGTTGTGTTCTTTGGT
TGTGGTGGGGGTGGTGTGTTGTGTGAGAACTGTATAGTGGACGCG
AGCATCTTTATTTTTTTGTTTTTTGTTGTGTGATACCGAACGCGC
CCGCACTTTGTGTGTGGGTTATAGTATTTTGTTTGTTGTTTTGTA
GGGCACACGGTGGATGCCTTGGCATATCAAGCCGATGAAGGACGT
GAGAGGCTGCGTTATGCCTCGAAGCTTGGCGTAATCATGGTCATA
GCTGTTTCCTGTGTGAAATTGTTATCCGCTCACAATTCCACACAA
CATACGAGCCGGAAGCATAAAGTGTAAAGCCTGGGGTGCCTAATG
AGTGAGCTAACTCACATTAATTGCGTTGCGCTCACTGCCCGCTTT
CCAGTCGGGAAACCTGTCGTGCCAGCTGCATTAATGAATCGGCCA
ACGCGCGGGGAGAGGCGGTTTGCGTATTGGGCGCTCTTCCGCTTC
CTCGCTCACTGACTCGCTGCGCTCGGTCGTTCGGCTGCGGCGAGC
GGTATCAGCTCACTCAAAGGCGGTAATACGGTTATCCACAGAATC
AGGGGATAACGCAGGAAAGAACATGTGAGCAAAAGGCCAGCAAAA
GGCCAGGAACCGTAAAAAGGCCGCGTTGCTGGCGTTTTTCCATAG
GCTCCGCCCCCCTGACGAGCATCACAAAAATCGACGCTCAAGTCA
GAGGTGGCGAAACCCGACAGGACTATAAAGATACCAGGCGTTTCC
CCCTGGAAGCTCCCTCGTGCGCTCTCCTGTTCCGACCCTGCCGCT
TACCGGATACCTGTCCGCCTTTCTCCCTTCGGGAAGCGTGGCGCT
TTCTCAATGCTCACGCTGTAGGTATCTCAGTTCGGTGTAGGTCGT
TCGCTCCAAGCTGGGCTGTGTGCACGAACCCCCCGTTCAGCCCGA
CCGCTGCGCCTTATCCGGTAACTATCGTCTTGAGTCCAACCCGGT
AAGACACGACTTATCGCCACTGGCAGCAGCCACTGGTAACAGGAT
TAGCAGAGCGAGGTATGTAGGCGGTGCTACAGAGTTCTTGAAGTG
GTGGCCTAACTACGGCTACACTAGAAGGACAGTATTTGGTATCTG
CGCTCTGCTGAAGCCAGTTACCTTCGGAAAAAGAGTTGGTAGCTC
TTGATCCGGCAAACAAACCACCGCTGGTAGCGGTGGTTTTTTTGT
TTGCAAGCAGCAGATTACGCGCAGAAAAAAAGGATCTCAAGAAGA
TCCTTTGATCTTTTCTACGGGGTCTGACGCTCAGTGGAACGAAAA
CTCACGTTAAGGGATTTTGGTCATGAGATTATCAAAAAGGATCTT
CACCTAGATCCTTTTGGGGTGGGCGAAGAACTCCAGCATGAGATC
CCCGCGCTGGAGGATCATCCAGCCCTGATAGAAACAGAAGCCACT
GGAGCACCTCAAAAACACCATCATACACTAAATCAGTAAGTTGGC
AGCATCACCCGACGCACTTTGCGCCGAATAAATACCTGTGACGGA
AGATCACTTCGCAGAATAAATAAATCCTGGTGTCCCTGTTGATAC
CGGGAAGCCCTGGGCCAACTTTTGGCGAAAATGAGACGTTGATCG
GCACGTAAGAGGTTCCAACTTTCACCATAATGAAATAAGATCACT
ACCGGGCGTATTTTTTGAGTTATCGAGATTTTCAGGAGCTGATAG
AAACAGAAGCCACTGGAGCACCTCAAAAACACCATCATACACTAA
ATCAGTAAGTTGGCAGCATCACCCGACGCACTTTGCGCCGAATAA
ATACCTGTGACGGAAGATCACTTCGCAGAATAAATAAATCCTGGT
GTCCCTGTTGATACCGGGAAGCCCTGGGCCAACTTTTGGCGAAAA
TGAGACGTTGATCGGCACGTAAGAGGTTCCAACTTTCACCATAAT
GAAATAAGATCACTACCGGGCGTATTTTTTGAGTTATCGAGATTT
TCAGGAGCTCTTTGGCATCGTCTCTCGCCTGTCCCCTCAGTTCAG
TAATTTCCTGCATTTGCCTGTTTCCAGTCGGTAGATATTCCACAA
AACAGCAGGGAAGCAGCGCTTTTCCGCTGCATAACCCTGCTTCGG
GGTCATTATAGCGATTTTTTCGGTATATCCATCCTTTTTCGCACG
ATATACAGGATTTTGCCAAAGGGTTCGTGTAGACTTTCCTTGGTG
TATCCAACGGCGTCAGCGGGGCAGGATAGGTGAAGTAGGCCCACC
CGCGAGCGGGTGTTCCTTCTTCACTGTCCCTTATTCGCACCTGGC
GGTGCTCAACGGGAATCCTGCTCTGCGAGGCTGGCCGGCTACCGC
CGGCGTAACAGATGAGGGCAAGCGGATGGCTGATGAAACCAAGCC
AACCAGGAAGGGCAGCCCACCTATCAAGGTGTACTGCCTTCCAGA
CGAACGAAGAGCGATTGAGGAAAAGGCGGCGGCGGCCGGCATGAG
CCTGTCGGCCTACCTGCTGGCCGTCGGCCAGGGCTACAAAATCAC
GGGCGTCGTGGACTATGAGCACGTCCGCGAGGGCGTCCCGGAAAA
CGATTCCGAAGCCCAACCTTTCATAGAAGGCGGCGGTGGAATCGA
AATCTCGTGATGGCAGGTTGGGCGTCGCTTGGTCGGTCATTTCGC
TCGGTACCCATCGGCATTTTCTTTTGCGTTTTTATTTGTTAACTG
TTAATTGTCCTTGTTCAAGGATGCTGTCTTTGACAACAGATGTTT
TCTTGCCTTTGATGTTCAGCAGGAAGCTCGGCGCAAACGTTGATT
GTTTGTCTGCGTAGAATCCTCTGTTTGTCATATAGCTTGTAATCA
CGACATTGTTTCCTTTCGCTTGAGGTACAGCGAAGTGTGAGTAAG
TAAAGGTTACATCGTTAGGATCAAGATCCATTTTTAACACAAGGC
CAGTTTTGTTCAGCGGCTTGTATGGGCCAGTTAAAGAATTAGAAA
CATAACCAAGCATGTAAATATCGTTAGACGTAATGCCGTCAATCG
TCATTTTTGATCCGCGGGAGTCAGTGAACAGGTACCATTTGCCGT
TCATTTTAAAGACGTTCGCGCGTTCAATTTCATCTGTTACTGTGT
TAGATGCAATCAGCGGTTTCATCACTTTTTTCAGTGTGTAATCAT
CGTTTAGCTCAATCATACCGAGAGCGCCGTTTGCTAACTCAGCCG
TGCGTTTTTTATCGCTTTGCAGAAGTTTTTGACTTTCTTGACGGA
AGAATGATGTGCTTTTGCCATAGTATGCTTTGTTAAATAAAGATT
CTTCGCCTTGGTAGCCATCTTCAGTTCCAGTGTTTGCTTCAAATA
CTAAGTATTTGTGGCCTTTATCTTCTACGTAGTGAGGATCTCTCA
GCGTATGGTTGTCGCCTGAGCTGTAGTTGCCTTCATCGATGAACT
GCTGTACATTTTGATACGTTTTTCCGTCACCGTCAAAGATTGATT
TATAATCCTCTACACCGTTGATGTTCAAAGAGCTGTCTGATGCTG
ATACGTTAACTTGTGCAGTTGTCAGTGTTTGTTTGCCGTAATGTT
TACCGGAGAAATCAGTGTAGAATAAACGGATTTTTCCGTCAGATG
TAAATGTGGCTGAACCTGACCATTCTTGTGTTTGGTCTTTTAGGA
TAGAATCATTTGCATCGAATTTGTCGCTGTCTTTAAAGACGCGGC
CAGCGTTTTTCCAGCTGTCAATAGAAGTTTCGCCGACTTTTTGAT
AGAACATGTAAATCGATGTGTCATCCGCATTTTTAGGATCTCCGG
CTAATGCAAAGACGATGTGGTAGCCGTGATAGTTTGCGACAGTGC
CGTCAGCGTTTTGTAATGGCCAGCTGTCCCAAACGTCCAGGCCTT
TTGCAGAAGAGATATTTTTAATTGTGGACGAATCAAATTCAGAAA
CTTGATATTTTTCATTTTTTTGCTGTTCAGGGATTTGCAGCATAT
CATGGCGTGTAATATGGGAAATGCCGTATGTTTCCTTATATGGCT
TTTGGTTCGTTTCTTTCGCAAACGCTTGAGTTGCGCCTCCTGCCA
GCAGTGCGGTAGTAAAGGTTAATACTGTTGCTTGTTTTGCAAACT
TTTTGATGTTCATCGTTCATGTCTCCTTTTTTATGTACTGTGTTA
GCGGTCTGCTTCTTCCAGCCCTCCTGTTTGAAGATGGCAAGTTAG
TTACGCACAATAAAAAAAGACCTAAAATATGTAAGGGGTGACGCC
AAAGTATACACTTTGCCCTTTACACATTTTAGGTCTTGCCTGCTT
TATCAGTAACAAACCCGCGCGATTTACTTTTCGACCTCATTCTAT
TAGACTCTCGTTTGGATTGCAACTGGTCTATTTTCCTCTTTTGTT
TGATAGAAAATCATAAAAGGATTTGCAGACTACGGGCCTAAAGAA
CTAAAAAATCTATCTGTTTCTTTTCATTCTCTGTATTTTTTATAG
TTTCTGTTGCATGGGCATAAAGTTGCCTTTTTAATCACAATTCAG
AAAATATCATAATATCTCATTTCACTAAATAATAGTGAACGGCAG
GTATATGTGATGGGTTAAAAAGGATCGATCCTCTAGCGAACCCCA
GAGTCCCGCTCAGAAGAACTCGTCAAGAAGGCGATAGAAGGCGAT
GCGCTGCGAATCGGGAGCGGCGATACCGTAAAGCACGAGGAAGCG
GTCAGCCCATTCGCCGCCAAGCTCTTCAGCAATATCACGGGTAGC
CAACGCTATGTCCTGATAGCGGTCCGCCACACCCAGCCGGCCACA
GTCGATGAATCCAGAAAAGCGGCCATTTTCCACCATGATATTCGG
CAAGCAGGCATCGCCATGGGTCACGACGAGATCCTCGCCGTCGGG
CATCCGCGCCTTGAGCCTGGCGAACAGTTCGGCTGGCGCGAGCCC
CTGATGCTCTTCGTCCAGATCATCCTGATCGACAAGACCGGCTTC
CATCCGAGTACGTGCTCGCTCGATGCGATGTTTCGCTTGGTGGTC
GAATGGGCAGGTAGCCGGATCAAGCGTATGCAGCCGCCGCATTGC
ATCAGCCATGATGGATACTTTCTCGGCAGGAGCAAGGTGAGATGA
CAGGAGATCCTGCCCCGGCACTTCGCCCAATAGCAGCCAGTCCCT
TCCCGCTTCAGTGACAACGTCGAGCACAGCTGCGCAAGGAACGCC
CGTCGTGGCCAGCCACGATAGCCGCGCTGCCTCGTCTTGGAGTTC
ATTCAGGGCACCGGACAGGTCGGTCTTGACAAAAAGAACCGGGCG
CCCCTGCGCTGACAGCCGGAACACGGCGGCATCAGAGCAGCCGAT
TGTCTGTTGTGCCCAGTCATAGCCGAATAGCCTCTCCACCCAAGC
GGCCGGAGAACCTGCGTGCAATCCATCTTGTTCAATCATGCGAAA
CGATCCTCATCCTGTCTCTTGATCAGATCTTGATCCCCTGCGCCA
TCAGATCCTTGGCGGCAAGAAAGCCATCCAGTTTACTTTGCAGGG
CTTCCCAACCTTACCAGAGGGCGCCCCAGCTGGCAATTCCGGTTC
GCTTGCTGTCCATAAAACCGCCCAGTCTAGCTATCGCCATGTAAG
CCCACTGCAAGCTACCTGCTTTCTCTTTGCGCTTGCGTTTTCCCT
TGTCCAGATAGCCCAGTAGCTGACATTCATCCGGGGTCAGCACCG
TTTCTGCGGACTGGCTTTCTACGTGTTCCGCTTCCTTTAGCAGCC
CTTGCGCCCTGAGTGCTTGCGGCAGCGTGAAGCTAGCTTATCGCG
CCATTCGCCATTCAGGCTGCGCAACTGTTGGGAAGGGCGATCGGT
GCGGGCCTCTTCGCTATTACGCCAGCTGGCGAAAGGGGGATGTGC
TGCAAGGCGATTAAGTTGGGTAACGCCAGGGTTTTCCCAGTCACG
ACGTTGTAAAACGACGGCCAGTGAATT
Target integration site (upstream):
SEQ ID NO: 81
CGCACAAGCGGCGGAGCATGTGGATTAATTCGATGCAACGCGAAG
AACCTTACCTGGGCTTGACATGGACCGGATCGGCGTAGAGATACG
TTTTCCCTTGTGGTCGGTTCACAGGTGGTGCATGGTTGTCGTCAG
CTCGTGTCGTGAGATGTTGGGTTAAGTCCCGCAACGAGCGCAACC
CTTGTCTTATGTTGCCAGCACATTGTGGTGGGTACTCATGAGAGA
CTGCCGGGGTTAACTCGGAGGAAGGTGGGGATGACGTCAAATCAT
CATGCCCCTTATGTCCAGGGCTTCACACATGCTACAATGGTCGGT
ACAGCGAGTTGCCACACCGTGAGGTGGAGCTAATCTCTTAAAGCC
GGCCTCAGTTCGGATTGGGGTCTGCAACTCGACCCCATGAAGTCG
GAGTCGCTAGTAATCGCAGATCAGCAACGCTGCGGTGAATACGTT
CCCGGGCCTTGTACACACCGCCCGTCACGTCATGAAAGTTGGTAA
CACCCGAAGCCAGTGGCCCAACCTTTTAGGGGGGAGCTGTCGAAG
GTGGGATCGGCGATTGGGACGAAGTCGTAACAAGGTAGCCGTACC
GGAAGGTGCGGCTGGA
Restriction site for verification of
positive integration
SEQ ID NO: 82
TCTAGA
Target integration site (downstream):
SEQ ID NO: 83
CCTCCTTTCTAAGGAGCTTTATTAACCCACATCAGACTGTGTCTG
GTTGGTGGGTTGTTGGTGTTGGAACCCGTATGTGGTTGCCATCAA
CATATTTTTAATCGGGTGGAGATGACCCCTCGGGTGACAACAACA
CAGCAAACAGTGCTGTGATTAATAGGTGGCATGCTGTTGGGTGTC
TGGAATGACATCGCAAGCATCACCTTTTGGTGGTGTGTGTGGGTT
GTTTCTAACATCGAGCATCGTCAACACGGGTAGAGAATGTTGTGT
TCTTTGGTTGTGGTGGGGGTGGTGTGTTGTGTGAGAACTGTATAG
TGGACGCGAGCATCTTTATTTTTTTGTTTTTTGTTGTGTGATACC
GAACGCGCCCGCACTTTGTGTGTGGGTTATAGTATTTTGTTTGTT
GTTTTGTAGGGCACACGGTGGATGCCTTGGCATATCAAGCCGATG
AAGGACGTGAGAGGCTGCGTTATGCCTCG

b) Integration of F30::broccoli at 3′ End of 16S rRNA of Corynebacterium glutamicum ATCC 13032 Δcg2273 Via Transformation and Selection Using Plasmid pK19msB_16S rRNA-broccoli

Competent cells of the C. glutamicum strain ATCC 13032 Δcg2273 were prepared and transformed by electroporation with pK19msB_16S rRNA-broccoli according to Tauch et al., 2002 (Tauch, A., Kirchner, O., Löffler, B., Götker, S., Pühler, A., and Kalinowski, J. Efficient electrotransformation of Corynebacterium diphtheriae with a Mini-Replicon Derived from the Corynebacterium glutamicum plasmid pGA1. Curr. Microbiol. 2002; 45, 362-367). The selection of the transformants was carried out on BHI (brain heart infusion) agar (1%) plates with 25 μg/ml of kanamycin. First and second recombination was conducted as previously described by Niebisch and Bott, 2001 (Niebisch and Bott. Molecular analysis of the cytochrome bc1-aa3 branch of the Corynebacterium glutamicum respiratory chain containing an unusual diheme cytochrome c1. Arch. Microbiol. 2001; 175, 282-294). Resulting clones were verified by colony-PCR using primers 16S rRNA-broccoli_for (SEQ ID NO: 84) and 16S rRNA-broccoli_rev (SEQ ID NO: 85). The resulting PCR product was digested by the restriction enzyme XbaI as only clones with successful integration of the aptamer are digestible by XbaI. The genome of C. glutamicum ATCC 13032 Δcg2273 Contains SIX Copies of rrn clusters (rrnA, rrnB, rrnC, rrnD, rrnE, rrnF) comprising each 16S rRNA, 23S rRNA and 5S rRNA (Martin, Barreiro, Gonzalez-Lavado, Barriuso. Ribosomal RNA and ribosomal proteins in corynebacteria. 2003. J Biotechnol. 4; 104(1-3):41-53). Due to the fact that all rrn clusters share a high sequence similarity, all six clusters are potential integration loci for F30::broccoli. To this end, strains, for which positive integration was shown in the first colony-PCR and by digestion, were tested again by colony-PCR using a universal primer (16S rRNA-broccoli_rev, SEQ ID NO: 85) and a cluster-specific primer (rrnA_rev SEQ ID NO: 86, rrnB_rev SEQ ID NO: 87, rrnC_rev SEQ ID NO: 88, rrnD_rev SEQ ID NO: 89, rrnE_rev SEQ ID NO: 90, rrnF_rev SEQ ID NO: 91). For further studies, a clone was used containing the 16S rRNA-F30::broccoli fusion at the 3′ end of the rrnA cluster. The resulting strain is named C. glutamicum ATCC 13032 Δcg2273_16S rRNA-broccoli.

16S rRNA-broccoli for:
SEQ ID NO: 84
GTCTTCCACGACTTCTGTGC
16S rRNA-broccoli_rev:
SEQ ID NO: 85
GTGTAAACCTCCACACCAGC
rrnA_rev:
SEQ ID NO: 86
CTTGACCTGGGAAGTTGCG
rrnB rev:
SEQ ID NO: 87
GCAAGATTGCTTGCTACCAC
rrnC_rev:
SEQ ID NO: 88
AGAAACTCGGAGCGACCATC
rrnD rev:
SEQ ID NO: 89
CGTCACACATCGCTCTACAG
rrnE_rev:
SEQ ID NO: 90
GAAGCCTTCCCATCAAGCATC
rrnF rev:
SEQ ID NO: 91
CACATCAAGGTGACACGGAG

### c) Cultivation and Validation of Cells Using Fluorescent Activated Cell Sorting (FACS)

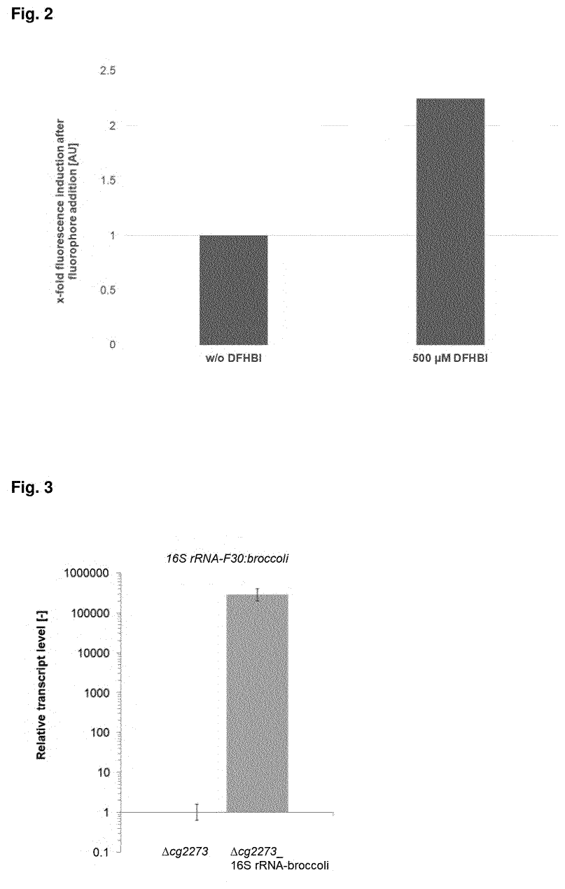

The produced strain C. glutamicum ATCC 13032 Δcg2273_16S rRNA-broccoli was streaked on BHI agar plates, which were cultivated at 30° C. overnight. Grown cells were resuspended in CGIII medium and the OD₆₀₀ was adjusted to 0.75 in a tube containing 2 mL CGIII cultivation medium. Cells were incubated at 30° C. and 120 rpm for four hours. Subsequently, cells were diluted to an OD₆₀₀ of 0.6 using PBS with a final concentration of 500 μM DFHBI. Cells were analyzed using an AriaIII High-speed cell sorter as already described in example 1d). DFHBI-stained cells showed a significantly increased fluorescent output compared to unstained cells (cf. FIG. 2).

d) Extraction of RNA and Quantification of Produced 16S rRNA-broccoli by Reverse Transcription Quantitative PCR

RNA was isolated according to example 1f) using 1.38×10⁹ cells per sample. Reverse transcription quantitative PCR (qPCR) was carried out according to the protocol of Wolf et al. (Wolf, Timo et al. (2017) The MaIR type regulator AcrC is a transcriptional repressor of acarbose biosynthetic genes in Actinoplanes sp. SE50/110, BMC Genomics) by use of the SensiFast SYBR No-Rox One-Step Kit (Bioline, London, United Kingdom) in 96 well Lightcycler® plates (Sarstedt, Numbrecht, Germany) in a LightCycler® 96 system of Roche (Mannheim, Germany) by use of the Lightcycler® 96 SW 1.1 (Roche, Mannheim, Germany). The relative RNA amount was calculated as 2^{-ΔCq}. ΔCq was calculated as the difference of the mean Cq of the strain C. glutamicum ATCC 13032 Δcg2273_16S rRNA-broccoli compared to the control strain C. glutamicum ATCC 13032 Δcg2273 without F30::broccoli integration in the genome. For qPCR, the primer pair qPCR_broc_fw (SEQ ID NO: 92) and qPCR_broc_rev (SEQ ID NO: 93) was used to amplify a 233 bp fragment incorporating the F30::broccoli fragment. The results show the relative transcript levels and verify the presence of 16S rRNA-F30::broccoli transcripts in the prepared RNA sample of strain C. glutamicum ATCC 13032 Δcg2273_16S rRNA-broccoli (cf. FIG. 3).

qPCR_broc_fw:
SEQ ID NO: 92
tcatgaaagttggtaacacccgaag
qPCR_broc_rev:
SEQ ID NO: 93
ttgccatgaatgatcccgaaggat

This experiment shows the successful linking of a hitherto unsuspicious phenotype (RNA production) with a fluorescence output. In this experiment, the produced RNA has a length of 1545 nucleotides and is transcribed from the chromosome of a gram-positive bacterial cell. In accordance with the procedure shown in example 1, the optimization of the fermentative production of long RNA encoded in a chromosomal locus is therefore possible using the invention.

### Example 3

a) Construction of the Vector pUC18_PT7-U1A-F30::broccoli/UUCG-TT7

The construction of the plasmid was achieved by means of chemical synthesis of the synthetic DNA-fragment (SEQ ID NO: 94 for PT7-U1A-F30::broccoli/UUCG-TT7) and its ligation into pUC18 resulting in plasmid pUC18-PT7-U1A-F30::broccoli/UUCG-TT7 (SEQ ID NO: 95, ordered from Twist Bioscience, South San Francisco, USA) (Norrander J, Kempe T, Messing J. Construction of improved M13 vectors using oligodeoxynucleotide-directed mutagenesis. Gene. 1983 December; 26(1):101-6.). SEQ ID NO: 94 contained the promoter P_{T7} (SEQ ID NO: 96), an RNA of interest (SEQ ID NO: 68), the F30 scaffold with a broccoli aptamer in the first integration point and a “UUCG spacer” in the second integration point (SEQ ID NO: 69) and a terminator sequence T_{T7} (SEQ ID NO: 97).

After cleavage of the synthesized DNA fragment with the restriction enzymes EcoRI and HindIII and subsequent purification of the reaction mixture, the DNA fragment that had been cut out was used for a ligation reaction with vector pUC18 that had also been linearized with EcoRI and HindIII and dephosphorylated. The ligation mixture was used directly to transform E. coli DH5a, and the selection of transformants was carried out on LB plates containing 100 μg/ml carbenicillin. 16 colonies, which grew on these plates and were therefore resistant to carbenicillin, were used for colony-PCR. The colony-PCR was performed with primers pUC18_check_f (SEQ ID NO: 98) and pUC18_check_rev (SEQ ID NO: 99) to analyze whether the synthesized fragment was inserted into vector pUC18. The analysis of colony PCR products on an agarose gel showed the expected PCR product with a size of 428 bp (pUC18-PT7-U1A-F30::broccoli/UUCG-TT7), whereupon three colonies were cultured for plasmid preparations in a larger scale. After 16 h of cultivation, these cultures were collected by centrifugation and the plasmid DNA was prepared. Two of these plasmid preparations were sequenced with the primers used in the colony-PCR and sequence of the inserts showed 100% identity with the expected sequence. The resulting plasmid was named pUC18-PT7-U1A-F30::broccoli/UUCG-TT7 (SEQ ID NO: 95).

EcoRI recognition site on 5′-end, HindIII
recognition site on 3′-end:
SEQ ID NO: 94
GAATTCTAATACGACTCACTATAGAGCGGGAGACCGCTCGACCTT
AGTTCTCCTGTTGCGGGGGAGTTCATGGGATCCAGGTACCCAGGG
CGAGGCTTATCCATTGCACTCCGGATGTGCTGACCCCTGTTGCCA
TGTGTATGTGGGAGACGGTCGGGTCCAGATATTCGTATCTGTCGA
GTAGAGTGTGGGCTCCCACATACTCTGATGATCCTTCGGGATCAT
TCATGGCAACTAGCATAACCCCTTGGGGCCTCTAAACGGGTCTTG
AGGGGTTTTTTGAAGCTT
pUC18-PT7-U1A-F30::broccoli/UUCG-TT7:
SEQ ID NO: 95
AATTCTAATACGACTCACTATAGAGCGGGAGACCGCTCGACCTTA
GTTCTCCTGTTGCGGGGGAGTTCATGGGATCCAGGTACCCAGGGC
GAGGCTTATCCATTGCACTCCGGATGTGCTGACCCCTGTTGCCAT
GTGTATGTGGGAGACGGTCGGGTCCAGATATTCGTATCTGTCGAG
TAGAGTGTGGGCTCCCACATACTCTGATGATCCTTCGGGATCATT
CATGGCAACTAGCATAACCCCTTGGGGCCTCTAAACGGGTCTTGA
GGGGTTTTTTGAAGCTTGGCACTGGCCGTCGTTTTACAACGTCGT
GACTGGGAAAACCCTGGCGTTACCCAACTTAATCGCCTTGCAGCA
CATCCCCCTTTCGCCAGCTGGCGTAATAGCGAAGAGGCCCGCACC
GATCGCCCTTCCCAACAGTTGCGCAGCCTGAATGGCGAATGGCGC
CTGATGCGGTATTTTCTCCTTACGCATCTGTGCGGTATTTCACAC
CGCATATGGTGCACTCTCAGTACAATCTGCTCTGATGCCGCATAG
TTAAGCCAGCCCCGACACCCGCCAACACCCGCTGACGCGCCCTGA
CGGGCTTGTCTGCTCCCGGCATCCGCTTACAGACAAGCTGTGACC
GTCTCCGGGAGCTGCATGTGTCAGAGGTTTTCACCGTCATCACCG
AAACGCGCGAGACGAAAGGGCCTCGTGATACGCCTATTTTTATAG
GTTAATGTCATGATAATAATGGTTTCTTAGACGTCAGGTGGCACT
TTTCGGGGAAATGTGCGCGGAACCCCTATTTGTTTATTTTTCTAA
ATACATTCAAATATGTATCCGCTCATGAGACAATAACCCTGATAA
ATGCTTCAATAATATTGAAAAAGGAAGAGTATGAGTATTCAACAT
TTCCGTGTCGCCCTTATTCCCTTTTTTGCGGCATTTTGCCTTCCT
GTTTTTGCTCACCCAGAAACGCTGGTGAAAGTAAAAGATGCTGAA
GATCAGTTGGGTGCACGAGTGGGTTACATCGAACTGGATCTCAAC
AGCGGTAAGATCCTTGAGAGTTTTCGCCCCGAAGAACGTTTTCCA
ATGATGAGCACTTTTAAAGTTCTGCTATGTGGCGCGGTATTATCC
CGTATTGACGCCGGGCAAGAGCAACTCGGTCGCCGCATACACTAT
TCTCAGAATGACTTGGTTGAGTACTCACCAGTCACAGAAAAGCAT
CTTACGGATGGCATGACAGTAAGAGAATTATGCAGTGCTGCCATA
ACCATGAGTGATAACACTGCGGCCAACTTACTTCTGACAACGATC
GGAGGACCGAAGGAGCTAACCGCTTTTTTGCACAACATGGGGGAT
CATGTAACTCGCCTTGATCGTTGGGAACCGGAGCTGAATGAAGCC
ATACCAAACGACGAGCGTGACACCACGATGCCTGTAGCAATGGCA
ACAACGTTGCGCAAACTATTAACTGGCGAACTACTTACTCTAGCT
TCCCGGCAACAATTAATAGACTGGATGGAGGCGGATAAAGTTGCA
GGACCACTTCTGCGCTCGGCCCTTCCGGCTGGCTGGTTTATTGCT
GATAAATCTGGAGCCGGTGAGCGTGGGTCTCGCGGTATCATTGCA
GCACTGGGGCCAGATGGTAAGCCCTCCCGTATCGTAGTTATCTAC
ACGACGGGGAGTCAGGCAACTATGGATGAACGAAATAGACAGATC
GCTGAGATAGGTGCCTCACTGATTAAGCATTGGTAACTGTCAGAC
CAAGTTTACTCATATATACTTTAGATTGATTTAAAACTTCATTTT
TAATTTAAAAGGATCTAGGTGAAGATCCTTTTTGATAATCTCATG
ACCAAAATCCCTTAACGTGAGTTTTCGTTCCACTGAGCGTCAGAC
CCCGTAGAAAAGATCAAAGGATCTTCTTGAGATCCTTTTTTTCTG
CGCGTAATCTGCTGCTTGCAAACAAAAAAACCACCGCTACCAGCG
GTGGTTTGTTTGCCGGATCAAGAGCTACCAACTCTTTTTCCGAAG
GTAACTGGCTTCAGCAGAGCGCAGATACCAAATACTGTCCTTCTA
GTGTAGCCGTAGTTAGGCCACCACTTCAAGAACTCTGTAGCACCG
CCTACATACCTCGCTCTGCTAATCCTGTTACCAGTGGCTGCTGCC
AGTGGCGATAAGTCGTGTCTTACCGGGTTGGACTCAAGACGATAG
TTACCGGATAAGGCGCAGCGGTCGGGCTGAACGGGGGGTTCGTGC
ACACAGCCCAGCTTGGAGCGAACGACCTACACCGAACTGAGATAC
CTACAGCGTGAGCTATGAGAAAGCGCCACGCTTCCCGAAGGGAGA
AAGGCGGACAGGTATCCGGTAAGCGGCAGGGTCGGAACAGGAGAG
CGCACGAGGGAGCTTCCAGGGGGAAACGCCTGGTATCTTTATAGT
CCTGTCGGGTTTCGCCACCTCTGACTTGAGCGTCGATTTTTGTGA
TGCTCGTCAGGGGGGCGGAGCCTATGGAAAAACGCCAGCAACGCG
GCCTTTTTACGGTTCCTGGCCTTTTGCTGGCCTTTTGCTCACATG
TTCTTTCCTGCGTTATCCCCTGATTCTGTGGATAACCGTATTACC
GCCTTTGAGTGAGCTGATACCGCTCGCCGCAGCCGAACGACCGAG
CGCAGCGAGTCAGTGAGCGAGGAAGCGGAAGAGCGCCCAATACGC
AAACCGCCTCTCCCCGCGCGTTGGCCGATTCATTAATGCAGCTGG
CACGACAGGTTTCCCGACTGGAAAGCGGGCAGTGAGCGCAACGCA
ATTAATGTGAGTTAGCTCACTCATTAGGCACCCCAGGCTTTACAC
TTTATGCTTCCGGCTCGTATGTTGTGTGGAATTGTGAGCGGATAA
CAATTTCACACAGGAAACAGCTATGACCATGATTACG
PT7:
SEQ ID NO: 96
TAATACGACTCACTATAG
TT7:
SEQ ID NO: 97
CTAGCATAACCCCTTGGGGCCTCTAAACGGGTCTTGAGGGGTTTT
TTG
pUC18_check f:
SEQ ID NO: 98
TTCCGGCTCGTATGTTGTG
pUC18_check_rev:
SEQ ID NO: 99
AGGCGATTAAGTTGGGTAACG

b) Transformation of E. coli HT115 with pUC18-PT7-U1A-F30::broccoli/UUCG-TT7

For transformation of plasmids in E. coli HT115 cells (Timmons, Court, Fire (2001) Ingestion of bacterially expressed dsRNAs can produce specific and potent genetic interference in Caenorhabditis elegans. Gene 263 (2001) 103-112), the transformation and storage solution (TSS) transformation protocol according to Chung et al., 1989 was used (Chung, Niemela, Miller (1989). One-step preparation of competent Escherichia coli: Transformation and storage of bacterial cells in the same solution. Proc Natl Acad Sci USA 86, 2172-2175). A single colony of the target strain was inoculated in a tube containing 3 mL LB medium and grown until an OD₆₀₀ between 0.3 and 0.8 was reached. Subsequently, the culture was chilled on ice for ten minutes. An equal volume (3 mL) of ice cold 2× TSS (8 g/L Bacto-Tryptone, 5 g/L Yeast Extract, 5 g/L NaCl, 200 g/L PEG 8000) was added and the tube was vortexed thoroughly by avoiding warming up the cells. The bacterial suspension was incubated for further ten minutes on ice. To 1 mL of competent cells at least 10 ng plasmid DNA were added and mixed by vortexing. The suspension was then left on ice for 30 minutes and 200 μL of the culture were plated on LB agar plates (1%) containing 100 μg/mL carbenicillin.

c) Cultivation and Phenotype Validation of E. coli HT115_pUC18_PT7-U1A-F30::broccoli/UUCG-TT7 Cells Using Fluorescence Activated Cell Sorting (FACS)

E. coli HT115_pUC18_PT7-U1A-F30::broccoli/UUCG-TT7 cells were inoculated from a single colony in a tube containing 2 mL 2× YT medium (16 g/L tryptone, 10 g/L yeast extract and 5 g/L NaCl) with 100 μg/mL carbenicillin and cultivated overnight at 37° C. and 120 rpm. The next day, the pre-culture was used to inoculate 2 mL fresh 2× YT medium containing 100 μg/mL carbenicillin to an OD₆₀₀ of 0.1. Cells were grown at 37° C. and 120 rpm and after three hours, expression of T7 RNA polymerase was induced by addition of 0.4 mM IPTG. Cultivation was continued for further four hours. Then, cells were diluted to an OD₆₀₀ of 0.6 using PBS with a final concentration of 50 μM DFHBI. After 10 min of incubation, the cell suspension was analyzed by FACS as described in example 1d). DFHBI-stained E. coli HT115_pUC18_PT7-U1A-F30::broccoli/UUCG-TT7 cells showed a significantly increased fluorescence in comparison to unstained cells.

This experiment shows the successful linking of a hitherto unsuspicious phenotype (RNA production) with a fluorescence output. In this experiment, the produced RNA is transcribed from a vector in a gram-negative bacterial cell. In accordance with the procedure shown in example 1, the optimization of the fermentative production of RNA, using a gram-negative bacterial cell, is therefore possible using the invention.

### Example 4

a) Construction of the Vector pJC1-PF1-U1A-F30::mango3-TF1

The construction of the plasmid was achieved by means of chemical synthesis of the synthetic DNA-fragment (SEQ ID NO: 100 for PF1-U1A-F30::mango3-TF1, ordered from Twist Bioscience, South San Francisco, USA) and its ligation into pJC1 (CREMER, J., TREPTOW, C., EGGELING, L., and SAHM, H. Regulation of Enzymes of Lysine Biosynthesis in Corynebacterium glutamicum. Microbiol. 1988; 134, 3221-3229). SEQ ID NO: 100 contained the promoter P_{F1} (SEQ ID NO: 67), an RNA of interest (SEQ ID NO: 68), the F30 scaffold with a mango3 aptamer in the integration point (SEQ ID NO: 101) and a terminator sequence T_{F1} (SEQ ID NO: 70).

After cleavage of the synthesized DNA fragment with the restriction enzymes XbaI and SalI and subsequent purification of the reaction mixture, the DNA fragment that had been cut out was used in a ligation reaction with vector pJC1 that had also been linearized with XbaI and SalI and dephosphorylated. The ligation mixture was used directly to transform E. coli DH5a, and the selection of transformants was carried out on LB plates containing 50 μg/ml kanamycin. 16 colonies, which grew on these plates and were therefore resistant to kanamycin, were used for colony PCR. The colony PCR was performed with primers pJC1_check_f (SEQ ID NO: 73) and pJC1_check_rev (SEQ ID NO: 74), to analyze whether the synthesized fragment was inserted into vector pJC1. The analysis of colony PCR products on an agarose gel showed the expected PCR product with a size of 682 bp (pJC1-PF1-U1A-F30::mango3-TF1) whereupon three colonies were cultured for plasmid preparations in a larger scale. After 16 h of cultivation, these cultures were collected by centrifugation and the plasmid DNA was prepared. Two of these plasmid preparations were sequenced with the primers used in the colony PCR and sequence of the inserts showed 100% identity with the expected sequence. The resulting plasmid was named pJC1-PF1-U1A-F30::mango3-TF1 (SEQ ID NO: 102).

recognition site on 5′-end, SalI recognition
site on 3′-end and a SacI recognition site
upstream of the sequence 101:
SEQ ID NO: 100
CTGTCTCTAGACTCGAGCGGGACGGTCGAACCAGCTTCAAGCGAC
CGGATGAGTATGTTACAGTAGATAGCGAGCGGGAGACCGCTCGAC
CTTAGTTCTCCTGTTGCGGGGGAGTTCATGGGATCCAGGTACCCA
GGGCGAGGCTTATCCATTGCACTCCGGATGTGCTGACCCCTGGGA
GCTCTTGCCATGTGTATGTGGGGGAAGGATTGGTATGTGGTATAC
CCACATACTCTGATGATCCTTCGGGATCATTCATGGCAAGCTAGC
ATAGCATAAAATAACGCCCCACCTTCTTAACGGGAGGTGGGGCGT
TATTTTTACGTCGAC
F30 with mango3 in its insertion site 1
and “TTCG” in insertion site 2:
SEQ ID NO: 101
TTGCCATGTGTATGTGGGGGAAGGATTGGTATGTGGTATACCCAC
ATACTCTGATGATCCTTCGGGATCATTCATGGCAA
pJC1-PF1-U1A-F30::mango3-TF1:
SEQ ID NO: 102
CTAGACTCGAGCGGGACGGTCGAACCAGCTTCAAGCGACCGGATG
AGTATGTTACAGTAGATAGCGAGCGGGAGACCGCTCGACCTTAGT
TCTCCTGTTGCGGGGGAGTTCATGGGATCCAGGTACCCAGGGCGA
GGCTTATCCATTGCACTCCGGATGTGCTGACCCCTGGGAGCTCTT
GCCATGTGTATGTGGGGGAAGGATTGGTATGTGGTATACCCACAT
ACTCTGATGATCCTTCGGGATCATTCATGGCAAGCTAGCATAGCA
TAAAATAACGCCCCACCTTCTTAACGGGAGGTGGGGCGTTATTTT
TACGTCGACCTGCAGCAATGGCAACAACGTTGCGCAAACTATTAA
CTGGCGAACTACTTACTCTAGCTTCCCGGCAACAATTAATAGACT
GGATGGAGGCGGATAAAGTTGCAGGACCACTTCTGCGCTCGGCCC
TTCCGGCTGGCTGGTTTATTGCTGATAAATCTGGAGCCGGTGAGC
GTGGGTCTCGCGGTATCATTGCAGCACTGGGGCCAGATGGTAAGC
CCTCCCGTATCGTAGTTATCTACACGACGGGGAGTCAGGCAACTA
TGGATGAACGAAATAGACAGATCGCTGAGATAGGTGCCTCACTGA
TTAAGCATTGGTAACTGTCAGACCAAGTTTACTCATATATACTTT
AGATTGATTTAAAACTTCATTTTTAATTTAAAAGGATCTAGGTGA
AGATCCTTTTTGATAATCTCATGACCAAAATCCCTTAACGTGAGT
TTTCGTTCCACTGAGCGTCAGACCCCTTAATAAGATGATCTTCTT
GAGATCGTTTTGGTCTGCGCGTAATCTCTTGCTCTGAAAACGAAA
AAACCGCCTTGCAGGGCGGTTTTTCGAAGGTTCTCTGAGCTACCA
ACTCTTTGAACCGAGGTAACTGGCTTGGAGGAGCGCAGTCACCAA
AACTTGTCCTTTCAGTTTAGCCTTAACCGGCGCATGACTTCAAGA
CTAACTCCTCTAAATCAATTACCAGTGGCTGCTGCCAGTGGTGCT
TTTGCATGTCTTTCCGGGTTGGACTCAAGACGATAGTTACCGGAT
AAGGCGCAGCGGTCGGACTGAACGGGGGGTTCGTGCATACAGTCC
AGCTTGGAGCGAACTGCCTACCCGGAACTGAGTGTCAGGCGTGGA
ATGAGACAAACGCGGCCATAACAGCGGAATGACACCGGTAAACCG
AAAGGCAGGAACAGGAGAGCGCACGAGGGAGCCGCCAGGGGGAAA
CGCCTGGTATCTTTATAGTCCTGTCGGGTTTCGCCACCACTGATT
TGAGCGTCAGATTTCGTGATGCTTGTCAGGGGGGCGGAGCCTATG
GAAAAACGGCTTTGCCGCGGCCCTCTCACTTCCCTGTTAAGTATC
TTCCTGGCATCTTCCAGGAAATCTCCGCCCCGTTCGTAAGCCATT
TCCGCTCGCCGCAGTCGAACGACCGAGCGTAGCGAGTCAGTGAGC
GAGGAAGCGGAATATATCCTGTATCACATATTCTGCTGACGCACC
GGTGCAGCCTTTTTTCTCCTGCCACATGAAGCACTTCACTGACAC
CCTCATCAGTGCCAACATAGTAAGCCAGTATACACTCCGCTAGCG
CTGAGGTCTGCCTCGTGAAGAAGGTGTTGCTGACTCATACCAGGC
CTGAATCGCCCCATCATCCAGCCAGAAAGTGAGGGAGCCACGGTT
GATGAGAGCTTTGTTGTAGGTGGACCAGTTGGTGATTTTGAACTT
TTGCTTTGCCACGGAACGGTCTGCGTTGTCGGGAAGATGCGTGAT
CTGATCCTTCAACTCAGCAAAAGTTCGATTTATTCAACAAAGCCA
CGTTGTGTCTCAAAATCTCTGATGTTACATTGCACAAGATAAAAA
TATATCATCATGAACAATAAAACTGTCTGCTTACATAAACAGTAA
TACAAGGGGTGTTATGAGCCATATTCAACGGGAAACGTCTTGCTC
GAGGCCGCGATTAAATTCCAACATGGATGCTGATTTATATGGGTA
TAAATGGGCTCGCGATAATGTCGGGCAATCAGGTGCGACAATCTA
TCGATTGTATGGGAAGCCCGATGCGCCAGAGTTGTTTCTGAAACA
TGGCAAAGGTAGCGTTGCCAATGATGTTACAGATGAGATGGTCAG
ACTAAACTGGCTGACGGAATTTATGCCTCTTCCGACCATCAAGCA
TTTTATCCGTACTCCTGATGATGCATGGTTACTCACCACTGCGAT
CCCCGGGAAAACAGCATTCCAGGTATTAGAAGAATATCCTGATTC
AGGTGAAAATATTGTTGATGCGCTGGCAGTGTTCCTGCGCCGGTT
GCATTCGATTCCTGTTTGTAATTGTCCTTTTAACAGCGATCGCGT
ATTTCGTCTCGCTCAGGCGCAATCACGAATGAATAACGGTTTGGT
TGATGCGAGTGATTTTGATGACGAGCGTAATGGCTGGCCTGTTGA
ACAAGTCTGGAAAGAAATGCATAAGCTTTTGCCATTCTCACCGGA
TTCAGTCGTCACTCATGGTGATTTCTCACTTGATAACCTTATTTT
TGACGAGGGGAAATTAATAGGTTGTATTGATGTTGGACGAGTCGG
AATCGCAGACCGATACCAGGATCTTGCCATCCTATGGAACTGCCT
CGGTGAGTTTTCTCCTTCATTACAGAAACGGCTTTTTCAAAAATA
TGGTATTGATAATCCTGATATGAATAAATTGCAGTTTCATTTGAT
GCTCGATGAGTTTTTCTAATCAGAATTGGTTAATTGGTTGTAACA
CTGGCAGAGCATTACGCTGACTTGACGGGACGGCGGCTTTGTTGA
ATAAATCGAACTTTTGCTGAGTTGAAGGATCAGATCACGCATCTT
CCCGACAACGCAGACCGTTCCGTGGCAAAGCAAAAGTTCAAAATC
ACCAACTGGTCCACCTACAACAAAGCTCTCATCAACCGTGGCTCC
CTCACTTTCTGGCTGGATGATGGGGCGATTCAGGCCTGGTATGAG
TCAGCAACACCTTCTTCACGAGGCAGACCTCAGCGCTCAAAGATG
CAGGGGTAAAAGCTAACCGCATCTTTACCGACAAGGCATCCGGCA
GTTCAACAGATCGGGAAGGGCTGGATTTGCTGAGGATGAAGGTGG
AGGAAGGTGATGTCATTCTGGTGAAGAAGCTCGACCGTCTTGGCC
GCGACACCGCCGACATGATCCAACTGATAAAAGAGTTTGATGCTC
AGGGTGTAGCGGTTCGGTTTATTGACGACGGGATCAGTACCGACG
GTGATATGGGGCAAATGGTGGTCACCATCCTGTCGGCTGTGGCAC
AGGCTGAACGCCGGAGGATCAAGTCGGTCAAGCCAAGCGCAACCA
GCGGCACCGCCGCGAGCAACGTCGCAAGGGCGATCAGGGGACGAT
TTTTGCGAAGAATTTCCACGGTAAGAATCCAATCTCTCGAATTTA
GGGTGAAAGAAGCTTGGCATAGGGGTGTGCACGAACTCGGTGGAG
GAAATTTCCGCGGGGCAAGGCTTCGCGAAGCGGAGTCGCGGCAGT
GGCTTTGAAGATCTTTGGGAGCAGTCCTTGTGCGCTTACGAGGTG
AGCCGGTGGGGAACCGTTATCTGCCTATGGTGTGAGCCCCCCTAG
AGAGCTTCAAGAGCAATCAGCCCGACCTAGAAAGGAGGCCAAGAG
AGAGACCCCTACGGGGGGAACCGTTTTCTGCCTACGAGATGGCAC
ATTTACTGGGAAGCTTTACGGCGTCCTCGTGGAAGTTCAATGCCC
GCAGACTTAAGTGCTCTATTCACGGTCTGACGTGACACGCTAAAT
TCAGACATAGCTTCATTGATTGTCGGCCACGAGCCAGTCTCTCCC
TCAACAGTCATAAACCAACCTGCAATGGTCAAGCGATTTCCTTTA
GCTTTCCTAGCTTGTCGTTGACTGGACTTAGCTAGTTTTTCTCGC
TGTGCTCGGGCGTACTCACTGTTTGGGTCTTTCCAGCGTTCTGCG
GCCTTTTTACCGCCACGTCTTCCCATAGTGGCCAGAGCTTTTCGC
CCTCGGCTGCTCTGCGTCTCTGTCTGACGAGCAGGGACGACTGGC
TGGCCTTTAGCGACGTAGCCGCGCACACGTCGCGCCATCGTCTGG
CGGTCACGCATCGGCGGCAGATCAGGCTCACGGCCGTCTGCTCCG
ACCGCCTGAGCGACGGTGTAGGCACGCTCGTAGGCGTCGATGATC
TTGGTGTCTTTTAGGCGCTCACCAGCCGCTTTTAACTGGTATCCC
ACAGTCAAAGCGTGGCGAAAAGCCGTCTCATCACGGGCGGCACGC
CCTGGAGCAGTCCAGAGGACACGGACGCCGTCGATCAGCTCTCCA
GACGCTTCAGCGGCGCTCGGCAGGCTTGCTTCAAGCGTGGCAAGT
GCTTTTGCTTCCGCAGTGGCTTTTCTTGCCGCTTCGATACGTGCC
CGTCCGCTAGAAAACTCCTGCTCATAGCGTTTTTTAGGTTTTTCT
GTGCCTGAGATCATGCGAGCAACCTCCATAAGATCAGCTAGGCGA
TCCACGCGATTGTGCTGGGCATGCCAGCGGTACGCGGTGGGATCG
TCGGAGACGTGCAGTGGCCACCGGCTCAGCCTATGTGAAAAAGCC
TGGTCAGCGCCGAAAACGCGGGTCATTTCCTCGGTCGTTGCAGCC
AGCAGGCGCATATTCGGGCTGCTCATGCCTGCTGCGGCATACACC
GGATCAATGAGCCAGATGAGCTGGCATTTCCCGCTCAGTGGATTC
ACGCCGATCCAAGCTGGCGCTTTTTCCAGGCGTGCCCAGCGCTCC
AAAATCGCGTAGACCTCGGGGTTTACGTGCTCGATTTTCCCGCCG
GCCTGGTGGCTCGGCACATCAATGTCCAGGACAAGCACGGCTGCG
TGCTGCGCGTGCGTCAGAGCAACATACTGGCACCGGGCAAGCGAT
TTTGAACCAACTCGGTATAACTTCGGCTGTGTTTCTCCCGTGTCC
GGGTCTTTGATCCAAGCGCTGGCGAAGTCGCGGGTCTTGCTGCCC
TGGAAATTTTCTCTGCCCAGGTGAGCGAGGAATTCGCGGCGGTCT
TCGCTCGTCCAGCCACGTGATCGCAGCGCGAGCTCGGGATGGGTG
TCGAACAGATCAGCGGAAAATTTCCAGGCCGGTGTGTCAATGTCT
CGTGAATCCGCTAGAGTCATTTTTGAGCGCTTTCTCCCAGGTTTG
GACTGGGGGTTAGCCGACGCCCTGTGAGTTACCGCTCACGGGGCG
TTCAACATTTTTCAGGTATTCGTGCAGCTTATCGCTTCTTGCCGC
CTGTGCGCTTTTTCGACGCGCGACGCTGCTGCCGATTCGGTGCAG
GTGGTGGCGGCGCTGACACGTCCTGGGCGGCCACGGCCACACGAA
ACGCGGCATTTACGATGTTTGTCATGCCTGCGGGCACCGCGCCAC
GATCGCGGATAATTCTCGCTGCCGCTTCCAGCTCTGTGACGACCA
TGGCCAAAATTTCGCTCGGGGGACGCACTTCCAGCGCCATTTGCG
ACCTAGCCGCCTCCAGCTCCTCGGCGTGGCGTTTGTTGGCGCGCT
CGCGGCTGGCTGCGGCACGACACGCATCTGAGCAATATTTTGCGC
GCCGTCCTCGCGGGTCAGGCCGGGGAGGAATCAGGCCACCGCAGT
AGGCGCAACTGATTCGATCCTCCACTACTGTGCGTCCTCCTGGCG
CTGCCGAGCACGCAGCTCGTCAGCCAGCTCCTCAAGATCCGCCAC
GAGAGTTTCTAGGTCGCTCGCGGCACTGGCCCAGTCTCGTGATGC
TGGCGCGTCCGTCGTATCGAGAGCTCGGAAAAATCCGATCACCGT
TTTTAAATCGACGGCAGCATCGAGCGCGTCGGACTCCAGCGCGAC
ATCAGAGAGATCCATAGCTGATGATTCGGGCCAATTTTGGTACTT
CGTCGTGAAGGTCATGACACCATTATAACGAACGTTCGTTAAAGT
TTTTGGCGGAAAATCACGCGGCACGAAAATTTTCACGAAGCGGGA
CTTTGCGCAGCTCAGGGGTGCTAAAAATTTTGTATCGCACTTGAT
TTTTCCGAAAGACAGATTATCTGCAAACGGTGTGTCGTATTTCTG
GCTTGGTTTTTAAAAAATCTGGAATCGAAAATTTGCGGGGCGACC
GAGAAGTTTTTTACAAAAGGCAAAAACTTTTTCGGGATCGACAGA
AATAAAACGATCGACGGTACGCAACAAAAAAGCGTCAGGATCGCC
GTAGAGCGATTGAAGACCGTCAACCAAAGGGGAAGCCTCCAATCG
ACGCGACGCGCGCTCTACGGCGATCCTGACGCAGATTTTTAGCTA
TCTGTCGCAGCGCCCTCAGGGACAAGCCACCCGCACAACGTCGCG
AGGGCGATCAGCGACGCCGCAGGGGGATCCT

b) Transformation of Corynebacterium glutamicum ATCC 13032 Δcg2273 with Plasmid pJC1-PF1-U1A-F30::mango3-TF1

Competent cells of the C. glutamicum strain ATCC 13032 Δcg2273 were prepared and transformed with pJC1-PF1-U1A-F30::mango3-TF1 according to Tauch et al., 2002 (Tauch, A., Kirchner, O., Löffler, B., Götker, S., Pühler, A., and Kalinowski, J. Efficient Electrotransformation of Corynebacterium diphtheriae with a Mini-Replicon Derived from the Corynebacterium glutamicum Plasmid pGA1. Curr. Microbiol. 2002; 45, 362-367). The selection of the transformants was carried out on CGIII (Menkel, E., Thierbach, G., Eggeling, L., and Sahm, H. Influence of increased aspartate availability on lysine formation by a recombinant strain of Corynebacterium glutamicum and utilization of fumarate. Appl. Environ. Microbiol. 1989; 55, 684-688) agar (1%) plates with 25 μg/ml of kanamycin. Clones thus obtained were named C. glutamicum ATCC 13032 Δcg2273 pJC1-PF1-U1A-F30::mango3-TF1.

### c) Cultivation of Cells and Phenotype Validation Using Fluorescent Activated Cell Sorting (FACS)

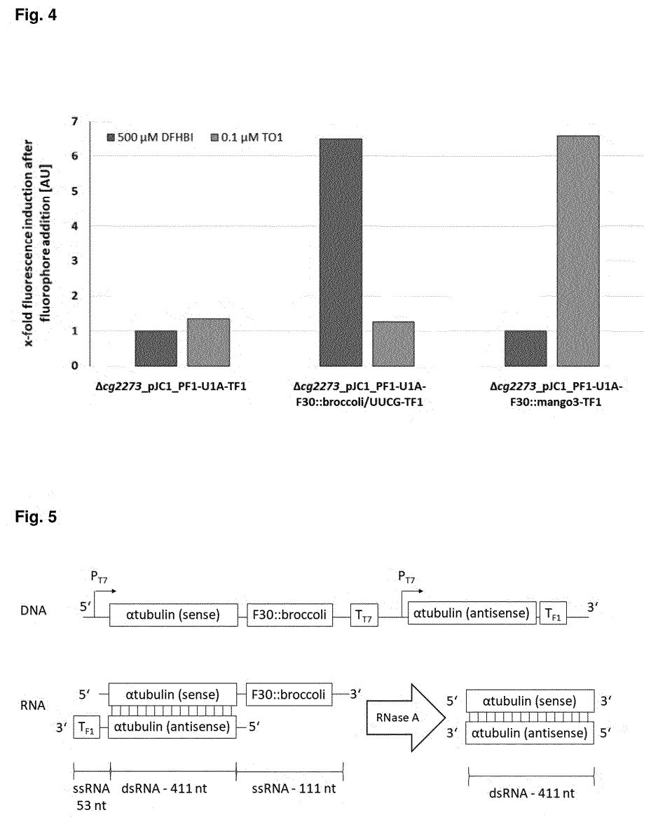

The produced strain C. glutamicum ATCC 13032 Δcg2273_pJC1-PF1-U1A-F30::mango3-TF1 as well as strains C. glutamicum ATCC 13032 Δcg2273_pJC1-PF1-U1A-F30::broccoli/UUCG-TF1 and C. glutamicum ATCC 13032 Δcg2273_pJC1-PF1-U1A-TF1 (see Example 1) were streaked on BHI agar plates containing 25 μg/mL kanamycin and cultivated at 30° C. Grown cells were resuspended in CGIII cultivation medium containing 25 μg/mL kanamycin and the OD₆₀₀ was adjusted to 0.75 in a tube containing 2 mL cultivation medium with 25 μg/mL kanamycin. Cells were incubated at 30° C. and 120 rpm for 18 hours. Subsequently, cells were diluted to an OD₆₀₀ of 0.6 using PBS with a final concentration of 500 μM DFHBI or 0.1 μM TO1. Stained and unstained cells were analyzed using an AriaIII High-speed cell sorter as already described in example 1d). DFHBI-stained C. glutamicum ATCC 13032 Δcg2273_pJC1-PF1-U1A-F30::broccoli/UUCG-TF1 cells showed an about six-fold increased fluorescent output compared to unstained cells (cf. FIG. 4). C. glutamicum ATCC 13032 Δcg2273 pJC1-PF1-U1A-F30::mango3-TF1 and C. glutamicum ATCC 13032 Δcg2273_pJC1-PF1-U1A-TF1 showed no increased fluorescent output after addition of the fluorophore DFHBI. TO1-stained C. glutamicum ATCC 13032 Δcg2273 pJC1-PF1-U1A-F30::mango3-TF1 cells showed an about six-fold increased fluorescent output compared to unstained cells (cf. FIG. 4). Upon addition of TO1, cells of strains C. glutamicum ATCC 13032 Δcg2273_pJC1-PF1-U1A-F30::broccoli/UUCG-TF1 and C. glutamicum ATCC 13032 Δcg2273_pJC1-PF1-U1A-TF1 showed a slight increase in fluorescence of about 1.2-fold compared to the control strains, which indicates a slightly reduced specificity for staining of cells using the fluorophore TO1.

This experiment shows the successful linking of a hitherto unsuspicious phenotype (RNA production) with a fluorescence output. In this experiment, the produced RNA is fused to an F30 scaffold and two different aptamers, broccoli or mango3. Fluorescence emission is induced by supplementation of the fluorophores DFHBI or TO1, respectively. In accordance with the procedure shown in example 1, the optimization of the fermentative production of RNA, using either of the two aptamers and their respective fluorophore, is therefore possible using the invention.

### Example 5

a) Construction of the Vector pJC1-PF1-U1A-F30::corn-TF1

The construction of the plasmid was achieved by means of chemical synthesis of the synthetic DNA-fragment (SEQ ID NO: 103 for PF1-U1A-F30::corn-TF1, ordered from Twist Bioscience, South San Francisco, USA) and its ligation into pJC1 (Cremer, J., Treptow, C., Eggeling, L., and Sahm, H. Regulation of Enzymes of Lysine Biosynthesis in Corynebacterium glutamicum. Microbiol. 1988; 134, 3221-3229) resulting in plasmid pJC1-PF1-U1A-F30::corn-TF1 (SEQ ID NO: cc3). SEQ ID NO: 103 contained the promoter P_{F1} (SEQ ID NO: 67), an RNA of interest (SEQ ID NO: 68), the F30 scaffold with a corn aptamer in the first integration point and UUCG in the second integration point (SEQ ID NO: 104) and a terminator sequence T_{F1} (SEQ ID NO: 70).

After cleavage of the synthesized DNA fragment with the restriction enzymes XbaI and SalI and subsequent purification of the reaction mixture, the DNA fragment that had been cut out was used in a ligation reaction with vector pJC1 that had also been linearized with XbaI and SalI and dephosphorylated. The ligation mixture was used directly to transform E. coli DH5a, and the selection of transformants was carried out on LB plates containing 50 μg/ml kanamycin. 16 colonies, which grew on these plates and were therefore resistant to kanamycin, were used for colony PCR. The colony PCR was performed with primers pJC1_check_f (SEQ ID NO: 73) and pJC1_check_rev (SEQ ID NO: 74), to analyze whether the synthesized fragment was inserted into vector pJC1. The analysis of colony PCR products on an agarose gel showed the expected PCR product with a size of 682 bp (pJC1-PF1-U1A-F30::corn-TF1) whereupon three colonies were cultured for plasmid preparations in a larger scale. After 16 h of cultivation, these cultures were collected by centrifugation and the plasmid DNA was prepared. Two of these plasmid preparations were sequenced with the primers used in the colony PCR and sequence of the inserts showed 100% identity with the expected sequence. The resulting plasmid was named pJC1-PF1-U1A-F30::corn-TF1 (SEQ ID NO: 105).

XbaI recognition site on 5′-end, SalI
recognition site on 3′-end:
SEQ ID NO: 103
CTGTCTCTAGACTCGAGCGGGACGGTCGAACCAGCTTCAAGCGAC
CGGATGAGTATGTTACAGTAGATAGCGAGCGGGAGACCGCTCGAC
CTTAGTTCTCCTGTTGCGGGGGAGTTCATGGGATCCAGGTACCCA
GGGCGAGGCTTATCCATTGCACTCCGGATGTGCTGACCCCTGGGA
GCTCTTGCCATGTGTATGTGGGCGAGGAAGGAGGTCTGAGGAGGT
CACTGCCCACATACTCTGATGATCCTTCGGGATCATTCATGGCAA
GCTAGCATAGCATAAAATAACGCCCCACCTTCTTAACGGGAGGTG
GGGCGTTATTTTTACGTCGAC
F30 with corn in its insertion site1 and “TTCG”
in insertion site2:
SEQ ID NO: 104
TTGCCATGTGTATGTGGGCGAGGAAGGAGGTCTGAGGAGGTCACT
GCCCACATACTCTGATGATCCTTCGGGATCATTCATGGCAA
pJC1-PF1-U1A-F30::corn-TF1
SEQ ID NO: 105
CTAGACTCGAGCGGGACGGTCGAACCAGCTTCAA
GCGACCGGATGAGTATGTTACAGTAGATAGCGAGCGGGAGACCGC
TCGACCTTAGTTCTCCTGTTGCGGGGGAGTTCATGGGATCCAGGT
ACCCAGGGCGAGGCTTATCCATTGCACTCCGGATGTGCTGACCCC
TGGGAGCTCTTGCCATGTGTATGTGGGCGAGGAAGGAGGTCTGAG
GAGGTCACTGCCCACATACTCTGATGATCCTTCGGGATCATTCAT
GGCAAGCTAGCATAGCATAAAATAACGCCCCACCTTCTTAACGGG
AGGTGGGGCGTTATTTTTACGTCGACCTGCAGCAATGGCAACAAC
GTTGCGCAAACTATTAACTGGCGAACTACTTACTCTAGCTTCCCG
GCAACAATTAATAGACTGGATGGAGGCGGATAAAGTTGCAGGACC
ACTTCTGCGCTCGGCCCTTCCGGCTGGCTGGTTTATTGCTGATAA
ATCTGGAGCCGGTGAGCGTGGGTCTCGCGGTATCATTGCAGCACT
GGGGCCAGATGGTAAGCCCTCCCGTATCGTAGTTATCTACACGAC
GGGGAGTCAGGCAACTATGGATGAACGAAATAGACAGATCGCTGA
GATAGGTGCCTCACTGATTAAGCATTGGTAACTGTCAGACCAAGT
TTACTCATATATACTTTAGATTGATTTAAAACTTCATTTTTAATT
TAAAAGGATCTAGGTGAAGATCCTTTTTGATAATCTCATGACCAA
AATCCCTTAACGTGAGTTTTCGTTCCACTGAGCGTCAGACCCCTT
AATAAGATGATCTTCTTGAGATCGTTTTGGTCTGCGCGTAATCTC
TTGCTCTGAAAACGAAAAAACCGCCTTGCAGGGCGGTTTTTCGAA
GGTTCTCTGAGCTACCAACTCTTTGAACCGAGGTAACTGGCTTGG
AGGAGCGCAGTCACCAAAACTTGTCCTTTCAGTTTAGCCTTAACC
GGCGCATGACTTCAAGACTAACTCCTCTAAATCAATTACCAGTGG
CTGCTGCCAGTGGTGCTTTTGCATGTCTTTCCGGGTTGGACTCAA
GACGATAGTTACCGGATAAGGCGCAGCGGTCGGACTGAACGGGGG
GTTCGTGCATACAGTCCAGCTTGGAGCGAACTGCCTACCCGGAAC
TGAGTGTCAGGCGTGGAATGAGACAAACGCGGCCATAACAGCGGA
ATGACACCGGTAAACCGAAAGGCAGGAACAGGAGAGCGCACGAGG
GAGCCGCCAGGGGGAAACGCCTGGTATCTTTATAGTCCTGTCGGG
TTTCGCCACCACTGATTTGAGCGTCAGATTTCGTGATGCTTGTCA
GGGGGGCGGAGCCTATGGAAAAACGGCTTTGCCGCGGCCCTCTCA
CTTCCCTGTTAAGTATCTTCCTGGCATCTTCCAGGAAATCTCCGC
CCCGTTCGTAAGCCATTTCCGCTCGCCGCAGTCGAACGACCGAGC
GTAGCGAGTCAGTGAGCGAGGAAGCGGAATATATCCTGTATCACA
TATTCTGCTGACGCACCGGTGCAGCCTTTTTTCTCCTGCCACATG
AAGCACTTCACTGACACCCTCATCAGTGCCAACATAGTAAGCCAG
TATACACTCCGCTAGCGCTGAGGTCTGCCTCGTGAAGAAGGTGTT
GCTGACTCATACCAGGCCTGAATCGCCCCATCATCCAGCCAGAAA
GTGAGGGAGCCACGGTTGATGAGAGCTTTGTTGTAGGTGGACCAG
TTGGTGATTTTGAACTTTTGCTTTGCCACGGAACGGTCTGCGTTG
TCGGGAAGATGCGTGATCTGATCCTTCAACTCAGCAAAAGTTCGA
TTTATTCAACAAAGCCACGTTGTGTCTCAAAATCTCTGATGTTAC
ATTGCACAAGATAAAAATATATCATCATGAACAATAAAACTGTCT
GCTTACATAAACAGTAATACAAGGGGTGTTATGAGCCATATTCAA
CGGGAAACGTCTTGCTCGAGGCCGCGATTAAATTCCAACATGGAT
GCTGATTTATATGGGTATAAATGGGCTCGCGATAATGTCGGGCAA
TCAGGTGCGACAATCTATCGATTGTATGGGAAGCCCGATGCGCCA
GAGTTGTTTCTGAAACATGGCAAAGGTAGCGTTGCCAATGATGTT
ACAGATGAGATGGTCAGACTAAACTGGCTGACGGAATTTATGCCT
CTTCCGACCATCAAGCATTTTATCCGTACTCCTGATGATGCATGG
TTACTCACCACTGCGATCCCCGGGAAAACAGCATTCCAGGTATTA
GAAGAATATCCTGATTCAGGTGAAAATATTGTTGATGCGCTGGCA
GTGTTCCTGCGCCGGTTGCATTCGATTCCTGTTTGTAATTGTCCT
TTTAACAGCGATCGCGTATTTCGTCTCGCTCAGGCGCAATCACGA
ATGAATAACGGTTTGGTTGATGCGAGTGATTTTGATGACGAGCGT
AATGGCTGGCCTGTTGAACAAGTCTGGAAAGAAATGCATAAGCTT
TTGCCATTCTCACCGGATTCAGTCGTCACTCATGGTGATTTCTCA
CTTGATAACCTTATTTTTGACGAGGGGAAATTAATAGGTTGTATT
GATGTTGGACGAGTCGGAATCGCAGACCGATACCAGGATCTTGCC
ATCCTATGGAACTGCCTCGGTGAGTTTTCTCCTTCATTACAGAAA
CGGCTTTTTCAAAAATATGGTATTGATAATCCTGATATGAATAAA
TTGCAGTTTCATTTGATGCTCGATGAGTTTTTCTAATCAGAATTG
GTTAATTGGTTGTAACACTGGCAGAGCATTACGCTGACTTGACGG
GACGGCGGCTTTGTTGAATAAATCGAACTTTTGCTGAGTTGAAGG
ATCAGATCACGCATCTTCCCGACAACGCAGACCGTTCCGTGGCAA
AGCAAAAGTTCAAAATCACCAACTGGTCCACCTACAACAAAGCTC
TCATCAACCGTGGCTCCCTCACTTTCTGGCTGGATGATGGGGCGA
TTCAGGCCTGGTATGAGTCAGCAACACCTTCTTCACGAGGCAGAC
CTCAGCGCTCAAAGATGCAGGGGTAAAAGCTAACCGCATCTTTAC
CGACAAGGCATCCGGCAGTTCAACAGATCGGGAAGGGCTGGATTT
GCTGAGGATGAAGGTGGAGGAAGGTGATGTCATTCTGGTGAAGAA
GCTCGACCGTCTTGGCCGCGACACCGCCGACATGATCCAACTGAT
AAAAGAGTTTGATGCTCAGGGTGTAGCGGTTCGGTTTATTGACGA
CGGGATCAGTACCGACGGTGATATGGGGCAAATGGTGGTCACCAT
CCTGTCGGCTGTGGCACAGGCTGAACGCCGGAGGATCAAGTCGGT
CAAGCCAAGCGCAACCAGCGGCACCGCCGCGAGCAACGTCGCAAG
GGCGATCAGGGGACGATTTTTGCGAAGAATTTCCACGGTAAGAAT
CCAATCTCTCGAATTTAGGGTGAAAGAAGCTTGGCATAGGGGTGT
GCACGAACTCGGTGGAGGAAATTTCCGCGGGGCAAGGCTTCGCGA
AGCGGAGTCGCGGCAGTGGCTTTGAAGATCTTTGGGAGCAGTCCT
TGTGCGCTTACGAGGTGAGCCGGTGGGGAACCGTTATCTGCCTAT
GGTGTGAGCCCCCCTAGAGAGCTTCAAGAGCAATCAGCCCGACCT
AGAAAGGAGGCCAAGAGAGAGACCCCTACGGGGGGAACCGTTTTC
TGCCTACGAGATGGCACATTTACTGGGAAGCTTTACGGCGTCCTC
GTGGAAGTTCAATGCCCGCAGACTTAAGTGCTCTATTCACGGTCT
GACGTGACACGCTAAATTCAGACATAGCTTCATTGATTGTCGGCC
ACGAGCCAGTCTCTCCCTCAACAGTCATAAACCAACCTGCAATGG
TCAAGCGATTTCCTTTAGCTTTCCTAGCTTGTCGTTGACTGGACT
TAGCTAGTTTTTCTCGCTGTGCTCGGGCGTACTCACTGTTTGGGT
CTTTCCAGCGTTCTGCGGCCTTTTTACCGCCACGTCTTCCCATAG
TGGCCAGAGCTTTTCGCCCTCGGCTGCTCTGCGTCTCTGTCTGAC
GAGCAGGGACGACTGGCTGGCCTTTAGCGACGTAGCCGCGCACAC
GTCGCGCCATCGTCTGGCGGTCACGCATCGGCGGCAGATCAGGCT
CACGGCCGTCTGCTCCGACCGCCTGAGCGACGGTGTAGGCACGCT
CGTAGGCGTCGATGATCTTGGTGTCTTTTAGGCGCTCACCAGCCG
CTTTTAACTGGTATCCCACAGTCAAAGCGTGGCGAAAAGCCGTCT
CATCACGGGCGGCACGCCCTGGAGCAGTCCAGAGGACACGGACGC
CGTCGATCAGCTCTCCAGACGCTTCAGCGGCGCTCGGCAGGCTTG
CTTCAAGCGTGGCAAGTGCTTTTGCTTCCGCAGTGGCTTTTCTTG
CCGCTTCGATACGTGCCCGTCCGCTAGAAAACTCCTGCTCATAGC
GTTTTTTAGGTTTTTCTGTGCCTGAGATCATGCGAGCAACCTCCA
TAAGATCAGCTAGGCGATCCACGCGATTGTGCTGGGCATGCCAGC
GGTACGCGGTGGGATCGTCGGAGACGTGCAGTGGCCACCGGCTCA
GCCTATGTGAAAAAGCCTGGTCAGCGCCGAAAACGCGGGTCATTT
CCTCGGTCGTTGCAGCCAGCAGGCGCATATTCGGGCTGCTCATGC
CTGCTGCGGCATACACCGGATCAATGAGCCAGATGAGCTGGCATT
TCCCGCTCAGTGGATTCACGCCGATCCAAGCTGGCGCTTTTTCCA
GGCGTGCCCAGCGCTCCAAAATCGCGTAGACCTCGGGGTTTACGT
GCTCGATTTTCCCGCCGGCCTGGTGGCTCGGCACATCAATGTCCA
GGACAAGCACGGCTGCGTGCTGCGCGTGCGTCAGAGCAACATACT
GGCACCGGGCAAGCGATTTTGAACCAACTCGGTATAACTTCGGCT
GTGTTTCTCCCGTGTCCGGGTCTTTGATCCAAGCGCTGGCGAAGT
CGCGGGTCTTGCTGCCCTGGAAATTTTCTCTGCCCAGGTGAGCGA
GGAATTCGCGGCGGTCTTCGCTCGTCCAGCCACGTGATCGCAGCG
CGAGCTCGGGATGGGTGTCGAACAGATCAGCGGAAAATTTCCAGG
CCGGTGTGTCAATGTCTCGTGAATCCGCTAGAGTCATTTTTGAGC
GCTTTCTCCCAGGTTTGGACTGGGGGTTAGCCGACGCCCTGTGAG
TTACCGCTCACGGGGCGTTCAACATTTTTCAGGTATTCGTGCAGC
TTATCGCTTCTTGCCGCCTGTGCGCTTTTTCGACGCGCGACGCTG
CTGCCGATTCGGTGCAGGTGGTGGCGGCGCTGACACGTCCTGGGC
GGCCACGGCCACACGAAACGCGGCATTTACGATGTTTGTCATGCC
TGCGGGCACCGCGCCACGATCGCGGATAATTCTCGCTGCCGCTTC
CAGCTCTGTGACGACCATGGCCAAAATTTCGCTCGGGGGACGCAC
TTCCAGCGCCATTTGCGACCTAGCCGCCTCCAGCTCCTCGGCGTG
GCGTTTGTTGGCGCGCTCGCGGCTGGCTGCGGCACGACACGCATC
TGAGCAATATTTTGCGCGCCGTCCTCGCGGGTCAGGCCGGGGAGG
AATCAGGCCACCGCAGTAGGCGCAACTGATTCGATCCTCCACTAC
TGTGCGTCCTCCTGGCGCTGCCGAGCACGCAGCTCGTCAGCCAGC
TCCTCAAGATCCGCCACGAGAGTTTCTAGGTCGCTCGCGGCACTG
GCCCAGTCTCGTGATGCTGGCGCGTCCGTCGTATCGAGAGCTCGG
AAAAATCCGATCACCGTTTTTAAATCGACGGCAGCATCGAGCGCG
TCGGACTCCAGCGCGACATCAGAGAGATCCATAGCTGATGATTCG
GGCCAATTTTGGTACTTCGTCGTGAAGGTCATGACACCATTATAA
CGAACGTTCGTTAAAGTTTTTGGCGGAAAATCACGCGGCACGAAA
ATTTTCACGAAGCGGGACTTTGCGCAGCTCAGGGGTGCTAAAAAT
TTTGTATCGCACTTGATTTTTCCGAAAGACAGATTATCTGCAAAC
GGTGTGTCGTATTTCTGGCTTGGTTTTTAAAAAATCTGGAATCGA
AAATTTGCGGGGCGACCGAGAAGTTTTTTACAAAAGGCAAAAACT
TTTTCGGGATCGACAGAAATAAAACGATCGACGGTACGCAACAAA
AAAGCGTCAGGATCGCCGTAGAGCGATTGAAGACCGTCAACCAAA
GGGGAAGCCTCCAATCGACGCGACGCGCGCTCTACGGCGATCCTG
ACGCAGATTTTTAGCTATCTGTCGCAGCGCCCTCAGGGACAAGCC
ACCCGCACAACGTCGCGAGGGCGATCAGCGACGCCGCAGGGGGAT
CCT

b) Transformation of Corynebacterium glutamicum ATCC 13032 Δcg2273 with Plasmid pJC1-PF1-U1A-F30::corn-TF1

Competent cells of the C. glutamicum strain ATCC 13032 Δcg2273 were prepared and transformed with pJC1-PF1-U1A-F30::corn-TF1 according to Tauch et al., 2002 (Tauch, A., Kirchner, O., Löffler, B., Götker, S., Pühler, A., and Kalinowski, J. Efficient Electrotransformation of Corynebacterium diphtheriae with a Mini-Replicon Derived from the Corynebacterium glutamicum Plasmid pGA1. Curr. Microbiol. 2002; 45, 362-367). The selection of the transformants was carried out on CGIII (Menkel, E., Thierbach, G., Eggeling, L., and Sahm, H. Influence of increased aspartate availability on lysine formation by a recombinant strain of Corynebacterium glutamicum and utilization of fumarate. Appl. Environ. Microbiol. 1989; 55, 684-688) agar (1%) plates with 25 μg/ml of kanamycin. Clones thus obtained were named C. glutamicum ATCC 13032 Δcg2273 pJC1-PF1-U1A-F30::corn-TF1.

### c) Cultivation of Cells and Phenotype Validation Using Fluorescent Activated Cell Sorting (FACS)

The produced strain C. glutamicum ATCC 13032 Δcg2273_pJC1-PF1-U1A-F30::corn-TF1 was streaked on BHI agar plates containing 25 μg/mL kanamycin and cultivated at 30° C. Grown cells were resuspended in CGIII cultivation medium containing 25 μg/mL kanamycin and the OD₆₀₀ was adjusted to 0.75 in a tube containing 2 mL cultivation medium with 25 μg/mL kanamycin. Cells were incubated at 30° C. and 120 rpm for 18 hours. Subsequently, cells were diluted to an OD₆₀₀ of 0.6 using PBS with a final concentration of 500 μM DFHO. Stained and unstained cells were analyzed using an AriaIII High-speed cell sorter as already described in example 1d). DFHO-stained C. glutamicum ATCC 13032 Δcg2273_pJC1-PF1-U1A-F30::corn-TF1 cells showed a significant increased fluorescent output compared to unstained cells.

This experiment shows the successful linking of a hitherto unsuspicious phenotype (RNA production) with a fluorescence output. In this experiment, the produced RNA is fused to an F30 scaffold and two different aptamers, broccoli or corn. Fluorescence emission is induced by supplementation of the fluorophores DFHBI or DFHO, respectively. In accordance with the procedure shown in example 1, the optimization of the fermentative production of RNA, using either of the two aptamers and their respective fluorophore, is therefore possible using the invention.

### Example 6

a) Construction of the Vectors pJC1_dsRNA_PT7-αTubulin-F30::broccoli and pJC1_dsRNA_PT7-CYP3-F30::broccoli

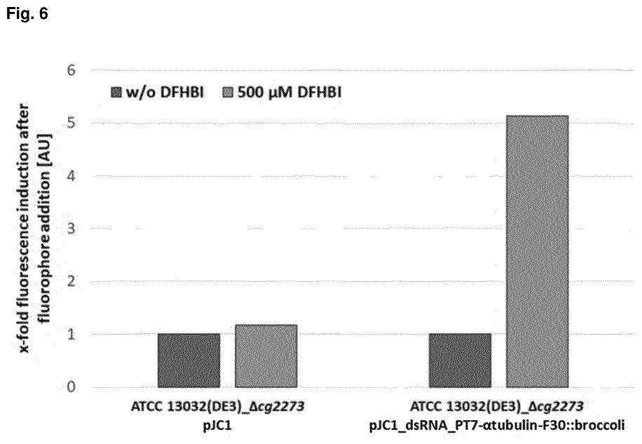

The construction of the plasmid was achieved by means of chemical synthesis of synthetic DNA-fragments (SEQ ID NO: 106 for dsRNA_PT7-αtubulin-F30::broccoli) and its ligation into restriction sites BamHI and EcoRI of vector pJC1 (SEQ ID NO: 107) (Cremer, J., Treptow, C., Eggeling, L., and Sahm, H. Regulation of Enzymes of Lysine Biosynthesis in Corynebacterium glutamicum. Microbiol. 1988; 134, 3221-3229) resulting in plasmids pJC1_dsRNA_PT7-αtubulin-F30::broccoli (SEQ ID NO: 108) (ordered from Twist Bioscience, South San Francisco, USA). SEQ ID NO: 106 contained the promoter P_{T}7 (SEQ ID NO: 96), a nucleotide sequence coding for 411 bp of the α-tubulin RNA from Varroa destructor (SEQ ID NO: 110) (Garbian et al., 2012, Bidirectional transfer of RNAi between Honey bee and Varroa destructor: Varroa gene silencing reduces Varroa population, PLOS Pathogens), the F30 scaffold with a broccoli aptamer in the first integration point and a “UUCG spacer” in the second integration point (SEQ ID NO: 69), a terminator sequence T_{T7} (SEQ ID NO: 97), a second T7 promoter (SEQ ID NO: 96), a nucleotide sequence coding for 411 bp of the α-tubulin antisense RNA (SEQ ID NO: 111) and a terminator sequence T_{F1} (SEQ ID NO: 70). The general principle of the design is depicted in FIG. 5.

dsRNA PT7-tubulin-F30::broccoli:
SEQ ID NO: 106
TAATACGACTCACTATAGGGCGAATGGAGAACATCGCACAGGACT
TCGGTAAAAAGTGCCGATTGGGCTTCGCCATCTACCCGGCTCCGC
AGGTTTCCACTGCCGTTGTCGAACCATACAACTCGGTTTTGACGA
CACATGCCACCCTCGAACACGCTGACTGCGTATTCATGATGGATA
ATGAGGCGATCTATCAGATCTGTCGTCGGAATCTTGGAGTTGAAC
GACCGGCGTATCAAAATCTCAATCGACTGATTAGCCAGGCCGTTT
CGGCGATAACCGCTTCTCTACGTTTTTCCGGAGCGTTGAATGTTG
ACCTCAACGAATTTCAGACGAATCTCGTCCCCTACCCGCGAATCC
ATTTCCCGCTCGTCACTTATGCTCCGATTATTTCGGCTGAGAAGG
CTCATCACGAGCAACATAACGTACTGGAATACGTATTGCCATGTG
TATGTGGGAGACGGTCGGGTCCAGATATTCGTATCTGTCGAGTAG
AGTGTGGGCTCCCACATACTCTGATGATCCTTCGGGATCATTCAT
GGCAAGTCGACCTAGCATAACCCCTTGGGGCCTCTAAACGGGTCT
TGAGGGGTTTTTTGCTGAAAGTATCATCCACATATGAGTACTCTA
ATACGACTCACTATAGGGCGATTCCAGTACGTTATGTTGCTCGTG
ATGAGCCTTCTCAGCCGAAATAATCGGAGCATAAGTGACGAGCGG
GAAATGGATTCGCGGGTAGGGGACGAGATTCGTCTGAAATTCGTT
GAGGTCAACATTCAACGCTCCGGAAAAACGTAGAGAAGCGGTTAT
CGCCGAAACGGCCTGGCTAATCAGTCGATTGAGATTTTGATACGC
CGGTCGTTCAACTCCAAGATTCCGACGACAGATCTGATAGATCGC
CTCATTATCCATCATGAATACGCAGTCAGCGTGTTCGAGGGTGGC
ATGTGTCGTCAAAACCGAGTTGTATGGTTCGACAACGGCAGTGGA
AACCTGCGGAGCCGGGTAGATGGCGAAGCCCAATCGGCACTTTTT
ACCGAAGTCCTGTGCGATGTTCTCCATATAGCATAAAATAACGCC
CCACCTTCTTAACGGGAGGTGGGGCGTTATTTTTA
pJC1
SEQ ID NO: 107
CCGAGAAGTTTTTTACAAAAGGCAAAAACTTTTTCGGGATCGACA
GAAATAAAACGATCGACGGTACGCAACAAAAAAGCGTCAGGATCG
CCGTAGAGCGATTGAAGACCGTCAACCAAAGGGGAAGCCTCCAAT
CGACGCGACGCGCGCTCTACGGCGATCCTGACGCAGATTTTTAGC
TATCTGTCGCAGCGCCCTCAGGGACAAGCCACCCGCACAACGTCG
CGAGGGCGATCAGCGACGCCGCAGGGGGATCCTCTAGAGTCGACC
TGCAGCAATGGCAACAACGTTGCGCAAACTATTAACTGGCGAACT
ACTTACTCTAGCTTCCCGGCAACAATTAATAGACTGGATGGAGGC
GGATAAAGTTGCAGGACCACTTCTGCGCTCGGCCCTTCCGGCTGG
CTGGTTTATTGCTGATAAATCTGGAGCCGGTGAGCGTGGGTCTCG
CGGTATCATTGCAGCACTGGGGCCAGATGGTAAGCCCTCCCGTAT
CGTAGTTATCTACACGACGGGGAGTCAGGCAACTATGGATGAACG
AAATAGACAGATCGCTGAGATAGGTGCCTCACTGATTAAGCATTG
GTAACTGTCAGACCAAGTTTACTCATATATACTTTAGATTGATTT
AAAACTTCATTTTTAATTTAAAAGGATCTAGGTGAAGATCCTTTT
TGATAATCTCATGACCAAAATCCCTTAACGTGAGTTTTCGTTCCA
CTGAGCGTCAGACCCCTTAATAAGATGATCTTCTTGAGATCGTTT
TGGTCTGCGCGTAATCTCTTGCTCTGAAAACGAAAAAACCGCCTT
GCAGGGCGGTTTTTCGAAGGTTCTCTGAGCTACCAACTCTTTGAA
CCGAGGTAACTGGCTTGGAGGAGCGCAGTCACCAAAACTTGTCCT
TTCAGTTTAGCCTTAACCGGCGCATGACTTCAAGACTAACTCCTC
TAAATCAATTACCAGTGGCTGCTGCCAGTGGTGCTTTTGCATGTC
TTTCCGGGTTGGACTCAAGACGATAGTTACCGGATAAGGCGCAGC
GGTCGGACTGAACGGGGGGTTCGTGCATACAGTCCAGCTTGGAGC
GAACTGCCTACCCGGAACTGAGTGTCAGGCGTGGAATGAGACAAA
CGCGGCCATAACAGCGGAATGACACCGGTAAACCGAAAGGCAGGA
ACAGGAGAGCGCACGAGGGAGCCGCCAGGGGGAAACGCCTGGTAT
CTTTATAGTCCTGTCGGGTTTCGCCACCACTGATTTGAGCGTCAG
ATTTCGTGATGCTTGTCAGGGGGGCGGAGCCTATGGAAAAACGGC
TTTGCCGCGGCCCTCTCACTTCCCTGTTAAGTATCTTCCTGGCAT
CTTCCAGGAAATCTCCGCCCCGTTCGTAAGCCATTTCCGCTCGCC
GCAGTCGAACGACCGAGCGTAGCGAGTCAGTGAGCGAGGAAGCGG
AATATATCCTGTATCACATATTCTGCTGACGCACCGGTGCAGCCT
TTTTTCTCCTGCCACATGAAGCACTTCACTGACACCCTCATCAGT
GCCAACATAGTAAGCCAGTATACACTCCGCTAGCGCTGAGGTCTG
CCTCGTGAAGAAGGTGTTGCTGACTCATACCAGGCCTGAATCGCC
CCATCATCCAGCCAGAAAGTGAGGGAGCCACGGTTGATGAGAGCT
TTGTTGTAGGTGGACCAGTTGGTGATTTTGAACTTTTGCTTTGCC
ACGGAACGGTCTGCGTTGTCGGGAAGATGCGTGATCTGATCCTTC
AACTCAGCAAAAGTTCGATTTATTCAACAAAGCCACGTTGTGTCT
CAAAATCTCTGATGTTACATTGCACAAGATAAAAATATATCATCA
TGAACAATAAAACTGTCTGCTTACATAAACAGTAATACAAGGGGT
GTTATGAGCCATATTCAACGGGAAACGTCTTGCTCGAGGCCGCGA
TTAAATTCCAACATGGATGCTGATTTATATGGGTATAAATGGGCT
CGCGATAATGTCGGGCAATCAGGTGCGACAATCTATCGATTGTAT
GGGAAGCCCGATGCGCCAGAGTTGTTTCTGAAACATGGCAAAGGT
AGCGTTGCCAATGATGTTACAGATGAGATGGTCAGACTAAACTGG
CTGACGGAATTTATGCCTCTTCCGACCATCAAGCATTTTATCCGT
ACTCCTGATGATGCATGGTTACTCACCACTGCGATCCCCGGGAAA
ACAGCATTCCAGGTATTAGAAGAATATCCTGATTCAGGTGAAAAT
ATTGTTGATGCGCTGGCAGTGTTCCTGCGCCGGTTGCATTCGATT
CCTGTTTGTAATTGTCCTTTTAACAGCGATCGCGTATTTCGTCTC
GCTCAGGCGCAATCACGAATGAATAACGGTTTGGTTGATGCGAGT
GATTTTGATGACGAGCGTAATGGCTGGCCTGTTGAACAAGTCTGG
AAAGAAATGCATAAGCTTTTGCCATTCTCACCGGATTCAGTCGTC
ACTCATGGTGATTTCTCACTTGATAACCTTATTTTTGACGAGGGG
AAATTAATAGGTTGTATTGATGTTGGACGAGTCGGAATCGCAGAC
CGATACCAGGATCTTGCCATCCTATGGAACTGCCTCGGTGAGTTT
TCTCCTTCATTACAGAAACGGCTTTTTCAAAAATATGGTATTGAT
AATCCTGATATGAATAAATTGCAGTTTCATTTGATGCTCGATGAG
TTTTTCTAATCAGAATTGGTTAATTGGTTGTAACACTGGCAGAGC
ATTACGCTGACTTGACGGGACGGCGGCTTTGTTGAATAAATCGAA
CTTTTGCTGAGTTGAAGGATCAGATCACGCATCTTCCCGACAACG
CAGACCGTTCCGTGGCAAAGCAAAAGTTCAAAATCACCAACTGGT
CCACCTACAACAAAGCTCTCATCAACCGTGGCTCCCTCACTTTCT
GGCTGGATGATGGGGCGATTCAGGCCTGGTATGAGTCAGCAACAC
CTTCTTCACGAGGCAGACCTCAGCGCTCAAAGATGCAGGGGTAAA
AGCTAACCGCATCTTTACCGACAAGGCATCCGGCAGTTCAACAGA
TCGGGAAGGGCTGGATTTGCTGAGGATGAAGGTGGAGGAAGGTGA
TGTCATTCTGGTGAAGAAGCTCGACCGTCTTGGCCGCGACACCGC
CGACATGATCCAACTGATAAAAGAGTTTGATGCTCAGGGTGTAGC
GGTTCGGTTTATTGACGACGGGATCAGTACCGACGGTGATATGGG
GCAAATGGTGGTCACCATCCTGTCGGCTGTGGCACAGGCTGAACG
CCGGAGGATCAAGTCGGTCAAGCCAAGCGCAACCAGCGGCACCGC
CGCGAGCAACGTCGCAAGGGCGATCAGGGGACGATTTTTGCGAAG
AATTTCCACGGTAAGAATCCAATCTCTCGAATTTAGGGTGAAAGA
AGCTTGGCATAGGGGTGTGCACGAACTCGGTGGAGGAAATTTCCG
CGGGGCAAGGCTTCGCGAAGCGGAGTCGCGGCAGTGGCTTTGAAG
ATCTTTGGGAGCAGTCCTTGTGCGCTTACGAGGTGAGCCGGTGGG
GAACCGTTATCTGCCTATGGTGTGAGCCCCCCTAGAGAGCTTCAA
GAGCAATCAGCCCGACCTAGAAAGGAGGCCAAGAGAGAGACCCCT
ACGGGGGGAACCGTTTTCTGCCTACGAGATGGCACATTTACTGGG
AAGCTTTACGGCGTCCTCGTGGAAGTTCAATGCCCGCAGACTTAA
GTGCTCTATTCACGGTCTGACGTGACACGCTAAATTCAGACATAG
CTTCATTGATTGTCGGCCACGAGCCAGTCTCTCCCTCAACAGTCA
TAAACCAACCTGCAATGGTCAAGCGATTTCCTTTAGCTTTCCTAG
CTTGTCGTTGACTGGACTTAGCTAGTTTTTCTCGCTGTGCTCGGG
CGTACTCACTGTTTGGGTCTTTCCAGCGTTCTGCGGCCTTTTTAC
CGCCACGTCTTCCCATAGTGGCCAGAGCTTTTCGCCCTCGGCTGC
TCTGCGTCTCTGTCTGACGAGCAGGGACGACTGGCTGGCCTTTAG
CGACGTAGCCGCGCACACGTCGCGCCATCGTCTGGCGGTCACGCA
TCGGCGGCAGATCAGGCTCACGGCCGTCTGCTCCGACCGCCTGAG
CGACGGTGTAGGCACGCTCGTAGGCGTCGATGATCTTGGTGTCTT
TTAGGCGCTCACCAGCCGCTTTTAACTGGTATCCCACAGTCAAAG
CGTGGCGAAAAGCCGTCTCATCACGGGCGGCACGCCCTGGAGCAG
TCCAGAGGACACGGACGCCGTCGATCAGCTCTCCAGACGCTTCAG
CGGCGCTCGGCAGGCTTGCTTCAAGCGTGGCAAGTGCTTTTGCTT
CCGCAGTGGCTTTTCTTGCCGCTTCGATACGTGCCCGTCCGCTAG
AAAACTCCTGCTCATAGCGTTTTTTAGGTTTTTCTGTGCCTGAGA
TCATGCGAGCAACCTCCATAAGATCAGCTAGGCGATCCACGCGAT
TGTGCTGGGCATGCCAGCGGTACGCGGTGGGATCGTCGGAGACGT
GCAGTGGCCACCGGCTCAGCCTATGTGAAAAAGCCTGGTCAGCGC
CGAAAACGCGGGTCATTTCCTCGGTCGTTGCAGCCAGCAGGCGCA
TATTCGGGCTGCTCATGCCTGCTGCGGCATACACCGGATCAATGA
GCCAGATGAGCTGGCATTTCCCGCTCAGTGGATTCACGCCGATCC
AAGCTGGCGCTTTTTCCAGGCGTGCCCAGCGCTCCAAAATCGCGT
AGACCTCGGGGTTTACGTGCTCGATTTTCCCGCCGGCCTGGTGGC
TCGGCACATCAATGTCCAGGACAAGCACGGCTGCGTGCTGCGCGT
GCGTCAGAGCAACATACTGGCACCGGGCAAGCGATTTTGAACCAA
CTCGGTATAACTTCGGCTGTGTTTCTCCCGTGTCCGGGTCTTTGA
TCCAAGCGCTGGCGAAGTCGCGGGTCTTGCTGCCCTGGAAATTTT
CTCTGCCCAGGTGAGCGAGGAATTCGCGGCGGTCTTCGCTCGTCC
AGCCACGTGATCGCAGCGCGAGCTCGGGATGGGTGTCGAACAGAT
CAGCGGAAAATTTCCAGGCCGGTGTGTCAATGTCTCGTGAATCCG
CTAGAGTCATTTTTGAGCGCTTTCTCCCAGGTTTGGACTGGGGGT
TAGCCGACGCCCTGTGAGTTACCGCTCACGGGGCGTTCAACATTT
TTCAGGTATTCGTGCAGCTTATCGCTTCTTGCCGCCTGTGCGCTT
TTTCGACGCGCGACGCTGCTGCCGATTCGGTGCAGGTGGTGGCGG
CGCTGACACGTCCTGGGCGGCCACGGCCACACGAAACGCGGCATT
TACGATGTTTGTCATGCCTGCGGGCACCGCGCCACGATCGCGGAT
AATTCTCGCTGCCGCTTCCAGCTCTGTGACGACCATGGCCAAAAT
TTCGCTCGGGGGACGCACTTCCAGCGCCATTTGCGACCTAGCCGC
CTCCAGCTCCTCGGCGTGGCGTTTGTTGGCGCGCTCGCGGCTGGC
TGCGGCACGACACGCATCTGAGCAATATTTTGCGCGCCGTCCTCG
CGGGTCAGGCCGGGGAGGAATCAGGCCACCGCAGTAGGCGCAACT
GATTCGATCCTCCACTACTGTGCGTCCTCCTGGCGCTGCCGAGCA
CGCAGCTCGTCAGCCAGCTCCTCAAGATCCGCCACGAGAGTTTCT
AGGTCGCTCGCGGCACTGGCCCAGTCTCGTGATGCTGGCGCGTCC
GTCGTATCGAGAGCTCGGAAAAATCCGATCACCGTTTTTAAATCG
ACGGCAGCATCGAGCGCGTCGGACTCCAGCGCGACATCAGAGAGA
TCCATAGCTGATGATTCGGGCCAATTTTGGTACTTCGTCGTGAAG
GTCATGACACCATTATAACGAACGTTCGTTAAAGTTTTTGGCGGA
AAATCACGCGGCACGAAAATTTTCACGAAGCGGGACTTTGCGCAG
CTCAGGGGTGCTAAAAATTTTGTATCGCACTTGATTTTTCCGAAA
GACAGATTATCTGCAAACGGTGTGTCGTATTTCTGGCTTGGTTTT
TAAAAAATCTGGAATCGAAAATTTGCGGGGCGA
pJC1_dsRNA_PT7-a-tubulin-F30 :: broccoli:
SEQ ID NO: 108
AACGTCTTGCTCGAGGCCGCGATTAAATTCCAACATGGATGCTGA
TTTATATGGGTATAAATGGGCTCGCGATAATGTCGGGCAATCAGG
TGCGACAATCTATCGATTGTATGGGAAGCCCGATGCGCCAGAGTT
GTTTCTGAAACATGGCAAAGGTAGCGTTGCCAATGATGTTACAGA
TGAGATGGTCAGACTAAACTGGCTGACGGAATTTATGCCTCTTCC
GACCATCAAGCATTTTATCCGTACTCCTGATGATGCATGGTTACT
CACCACTGCGATCCCCGGGAAAACAGCATTCCAGGTATTAGAAGA
ATATCCTGATTCAGGTGAAAATATTGTTGATGCGCTGGCAGTGTT
CCTGCGCCGGTTGCATTCGATTCCTGTTTGTAATTGTCCTTTTAA
CAGCGATCGCGTATTTCGTCTCGCTCAGGCGCAATCACGAATGAA
TAACGGTTTGGTTGATGCGAGTGATTTTGATGACGAGCGTAATGG
CTGGCCTGTTGAACAAGTCTGGAAAGAAATGCATAAGCTTTTGCC
ATTCTCACCGGATTCAGTCGTCACTCATGGTGATTTCTCACTTGA
TAACCTTATTTTTGACGAGGGGAAATTAATAGGTTGTATTGATGT
TGGACGAGTCGGAATCGCAGACCGATACCAGGATCTTGCCATCCT
ATGGAACTGCCTCGGTGAGTTTTCTCCTTCATTACAGAAACGGCT
TTTTCAAAAATATGGTATTGATAATCCTGATATGAATAAATTGCA
GTTTCATTTGATGCTCGATGAGTTTTTCTAATCAGAATTGGTTAA
TTGGTTGTAACACTGGCAGAGCATTACGCTGACTTGACGGGACGG
CGGCTTTGTTGAATAAATCGAACTTTTGCTGAGTTGAAGGATCAG
ATCACGCATCTTCCCGACAACGCAGACCGTTCCGTGGCAAAGCAA
AAGTTCAAAATCACCAACTGGTCCACCTACAACAAAGCTCTCATC
AACCGTGGCTCCCTCACTTTCTGGCTGGATGATGGGGCGATTCAG
GCCTGGTATGAGTCAGCAACACCTTCTTCACGAGGCAGACCTCAG
CGCTCAAAGATGCAGGGGTAAAAGCTAACCGCATCTTTACCGACA
AGGCATCCGGCAGTTCAACAGATCGGGAAGGGCTGGATTTGCTGA
GGATGAAGGTGGAGGAAGGTGATGTCATTCTGGTGAAGAAGCTCG
ACCGTCTTGGCCGCGACACCGCCGACATGATCCAACTGATAAAAG
AGTTTGATGCTCAGGGTGTAGCGGTTCGGTTTATTGACGACGGGA
TCAGTACCGACGGTGATATGGGGCAAATGGTGGTCACCATCCTGT
CGGCTGTGGCACAGGCTGAACGCCGGAGGATCAAGTCGGTCAAGC
CAAGCGCAACCAGCGGCACCGCCGCGAGCAACGTCGCAAGGGCGA
TCAGGGGACGATTTTTGCGAAGAATTTCCACGGTAAGAATCCAAT
CTCTCGAATTTAGGGTGAAAGAAGCTTGGCATAGGGGTGTGCACG
AACTCGGTGGAGGAAATTTCCGCGGGGCAAGGCTTCGCGAAGCGG
AGTCGCGGCAGTGGCTTTGAAGATCTTTGGGAGCAGTCCTTGTGC
GCTTACGAGGTGAGCCGGTGGGGAACCGTTATCTGCCTATGGTGT
GAGCCCCCCTAGAGAGCTTCAAGAGCAATCAGCCCGACCTAGAAA
GGAGGCCAAGAGAGAGACCCCTACGGGGGGAACCGTTTTCTGCCT
ACGAGATGGCACATTTACTGGGAAGCTTTACGGCGTCCTCGTGGA
AGTTCAATGCCCGCAGACTTAAGTGCTCTATTCACGGTCTGACGT
GACACGCTAAATTCAGACATAGCTTCATTGATTGTCGGCCACGAG
CCAGTCTCTCCCTCAACAGTCATAAACCAACCTGCAATGGTCAAG
CGATTTCCTTTAGCTTTCCTAGCTTGTCGTTGACTGGACTTAGCT
AGTTTTTCTCGCTGTGCTCGGGCGTACTCACTGTTTGGGTCTTTC
CAGCGTTCTGCGGCCTTTTTACCGCCACGTCTTCCCATAGTGGCC
AGAGCTTTTCGCCCTCGGCTGCTCTGCGTCTCTGTCTGACGAGCA
GGGACGACTGGCTGGCCTTTAGCGACGTAGCCGCGCACACGTCGC
GCCATCGTCTGGCGGTCACGCATCGGCGGCAGATCAGGCTCACGG
CCGTCTGCTCCGACCGCCTGAGCGACGGTGTAGGCACGCTCGTAG
GCGTCGATGATCTTGGTGTCTTTTAGGCGCTCACCAGCCGCTTTT
AACTGGTATCCCACAGTCAAAGCGTGGCGAAAAGCCGTCTCATCA
CGGGCGGCACGCCCTGGAGCAGTCCAGAGGACACGGACGCCGTCG
ATCAGCTCTCCAGACGCTTCAGCGGCGCTCGGCAGGCTTGCTTCA
AGCGTGGCAAGTGCTTTTGCTTCCGCAGTGGCTTTTCTTGCCGCT
TCGATACGTGCCCGTCCGCTAGAAAACTCCTGCTCATAGCGTTTT
TTAGGTTTTTCTGTGCCTGAGATCATGCGAGCAACCTCCATAAGA
TCAGCTAGGCGATCCACGCGATTGTGCTGGGCATGCCAGCGGTAC
GCGGTGGGATCGTCGGAGACGTGCAGTGGCCACCGGCTCAGCCTA
TGTGAAAAAGCCTGGTCAGCGCCGAAAACGCGGGTCATTTCCTCG
GTCGTTGCAGCCAGCAGGCGCATATTCGGGCTGCTCATGCCTGCT
GCGGCATACACCGGATCAATGAGCCAGATGAGCTGGCATTTCCCG
CTCAGTGGATTCACGCCGATCCAAGCTGGCGCTTTTTCCAGGCGT
GCCCAGCGCTCCAAAATCGCGTAGACCTCGGGGTTTACGTGCTCG
ATTTTCCCGCCGGCCTGGTGGCTCGGCACATCAATGTCCAGGACA
AGCACGGCTGCGTGCTGCGCGTGCGTCAGAGCAACATACTGGCAC
CGGGCAAGCGATTTTGAACCAACTCGGTATAACTTCGGCTGTGTT
TCTCCCGTGTCCGGGTCTTTGATCCAAGCGCTGGCGAAGTCGCGG
GTCTTGCTGCCCTGGAAATTTTCTCTGCCCAGGTGAGCGAGGAAT
TCGCGGCGGTCTTCGCTCGTCCAGCCACGTGATCGCAGCGCGAGC
TCGGGATGGGTGTCGAACAGATCAGCGGAAAATTTCCAGGCCGGT
GTGTCAATGTCTCGTGAATCCGCTAGAGTCATTTTTGAGCGCTTT
CTCCCAGGTTTGGACTGGGGGTTAGCCGACGCCCTGTGAGTTACC
GCTCACGGGGCGTTCAACATTTTTCAGGTATTCGTGCAGCTTATC
GCTTCTTGCCGCCTGTGCGCTTTTTCGACGCGCGACGCTGCTGCC
GATTCGGTGCAGGTGGTGGCGGCGCTGACACGTCCTGGGCGGCCA
CGGCCACACGAAACGCGGCATTTACGATGTTTGTCATGCCTGCGG
GCACCGCGCCACGATCGCGGATAATTCTCGCTGCCGCTTCCAGCT
CTGTGACGACCATGGCCAAAATTTCGCTCGGGGGACGCACTTCCA
GCGCCATTTGCGACCTAGCCGCCTCCAGCTCCTCGGCGTGGCGTT
TGTTGGCGCGCTCGCGGCTGGCTGCGGCACGACACGCATCTGAGC
AATATTTTGCGCGCCGTCCTCGCGGGTCAGGCCGGGGAGGAATCA
GGCCACCGCAGTAGGCGCAACTGATTCGATCCTCCACTACTGTGC
GTCCTCCTGGCGCTGCCGAGCACGCAGCTCGTCAGCCAGCTCCTC
AAGATCCGCCACGAGAGTTTCTAGGTCGCTCGCGGCACTGGCCCA
GTCTCGTGATGCTGGCGCGTCCGTCGTATCGAGAGCTCGGAAAAA
TCCGATCACCGTTTTTAAATCGACGGCAGCATCGAGCGCGTCGGA
CTCCAGCGCGACATCAGAGAGATCCATAGCTGATGATTCGGGCCA
ATTTTGGTACTTCGTCGTGAAGGTCATGACACCATTATAACGAAC
GTTCGTTAAAGTTTTTGGCGGAAAATCACGCGGCACGAAAATTTT
CACGAAGCGGGACTTTGCGCAGCTCAGGGGTGCTAAAAATTTTGT
ATCGCACTTGATTTTTCCGAAAGACAGATTATCTGCAAACGGTGT
GTCGTATTTCTGGCTTGGTTTTTAAAAAATCTGGAATCGAAAATT
TGCGGGGCGACCGAGAAGTTTTTTACAAAAGGCAAAAACTTTTTC
GGGATCGACAGAAATAAAACGATCGACGGTACGCAACAAAAAAGC
GTCAGGATCGCCGTAGAGCGATTGAAGACCGTCAACCAAAGGGGA
AGCCTCCAATCGACGCGACGCGCGCTCTACGGCGATCCTGACGCA
GATTTTTAGCTATCTGTCGCAGCGCCCTCAGGGACAAGCCACCCG
CACAACGTCGCGAGGGCGATCAGCGACGCCGCAGGGGGATCCGGA
TCCTCTAGACTAATACGACTCACTATAGGGCGAATGGAGAACATC
GCACAGGACTTCGGTAAAAAGTGCCGATTGGGCTTCGCCATCTAC
CCGGCTCCGCAGGTTTCCACTGCCGTTGTCGAACCATACAACTCG
GTTTTGACGACACATGCCACCCTCGAACACGCTGACTGCGTATTC
ATGATGGATAATGAGGCGATCTATCAGATCTGTCGTCGGAATCTT
GGAGTTGAACGACCGGCGTATCAAAATCTCAATCGACTGATTAGC
CAGGCCGTTTCGGCGATAACCGCTTCTCTACGTTTTTCCGGAGCG
TTGAATGTTGACCTCAACGAATTTCAGACGAATCTCGTCCCCTAC
CCGCGAATCCATTTCCCGCTCGTCACTTATGCTCCGATTATTTCG
GCTGAGAAGGCTCATCACGAGCAACATAACGTACTGGAATACGTA
TTGCCATGTGTATGTGGGAGACGGTCGGGTCCAGATATTCGTATC
TGTCGAGTAGAGTGTGGGCTCCCACATACTCTGATGATCCTTCGG
GATCATTCATGGCAAGTCGACCTAGCATAACCCCTTGGGGCCTCT
AAACGGGTCTTGAGGGGTTTTTTGCTGAAAGTATCATCCACATAT
GAGTACTCTAATACGACTCACTATAGGGCGATTCCAGTACGTTAT
GTTGCTCGTGATGAGCCTTCTCAGCCGAAATAATCGGAGCATAAG
TGACGAGCGGGAAATGGATTCGCGGGTAGGGGACGAGATTCGTCT
GAAATTCGTTGAGGTCAACATTCAACGCTCCGGAAAAACGTAGAG
AAGCGGTTATCGCCGAAACGGCCTGGCTAATCAGTCGATTGAGAT
TTTGATACGCCGGTCGTTCAACTCCAAGATTCCGACGACAGATCT
GATAGATCGCCTCATTATCCATCATGAATACGCAGTCAGCGTGTT
CGAGGGTGGCATGTGTCGTCAAAACCGAGTTGTATGGTTCGACAA
CGGCAGTGGAAACCTGCGGAGCCGGGTAGATGGCGAAGCCCAATC
GGCACTTTTTACCGAAGTCCTGTGCGATGTTCTCCATATAGCATA
AAATAACGCCCCACCTTCTTAACGGGAGGTGGGGCGTTATTTTTA
GGTACCGATATCGATATCCAATGGCAACAACGTTGCGCAAACTAT
TAACTGGCGAACTACTTACTCTAGCTTCCCGGCAACAATTAATAG
ACTGGATGGAGGCGGATAAAGTTGCAGGACCACTTCTGCGCTCGG
CCCTTCCGGCTGGCTGGTTTATTGCTGATAAATCTGGAGCCGGTG
AGCGTGGGTCTCGCGGTATCATTGCAGCACTGGGGCCAGATGGTA
AGCCCTCCCGTATCGTAGTTATCTACACGACGGGGAGTCAGGCAA
CTATGGATGAACGAAATAGACAGATCGCTGAGATAGGTGCCTCAC
TGATTAAGCATTGGTAACTGTCAGACCAAGTTTACTCATATATAC
TTTAGATTGATTTAAAACTTCATTTTTAATTTAAAAGGATCTAGG
TGAAGATCCTTTTTGATAATCTCATGACCAAAATCCCTTAACGTG
AGTTTTCGTTCCACTGAGCGTCAGACCCCTTAATAAGATGATCTT
CTTGAGATCGTTTTGGTCTGCGCGTAATCTCTTGCTCTGAAAACG
AAAAAACCGCCTTGCAGGGCGGTTTTTCGAAGGTTCTCTGAGCTA
CCAACTCTTTGAACCGAGGTAACTGGCTTGGAGGAGCGCAGTCAC
CAAAACTTGTCCTTTCAGTTTAGCCTTAACCGGCGCATGACTTCA
AGACTAACTCCTCTAAATCAATTACCAGTGGCTGCTGCCAGTGGT
GCTTTTGCATGTCTTTCCGGGTTGGACTCAAGACGATAGTTACCG
GATAAGGCGCAGCGGTCGGACTGAACGGGGGGTTCGTGCATACAG
TCCAGCTTGGAGCGAACTGCCTACCCGGAACTGAGTGTCAGGCGT
GGAATGAGACAAACGCGGCCATAACAGCGGAATGACACCGGTAAA
CCGAAAGGCAGGAACAGGAGAGCGCACGAGGGAGCCGCCAGGGGG
AAACGCCTGGTATCTTTATAGTCCTGTCGGGTTTCGCCACCACTG
ATTTGAGCGTCAGATTTCGTGATGCTTGTCAGGGGGGCGGAGCCT
ATGGAAAAACGGCTTTGCCGCGGCCCTCTCACTTCCCTGTTAAGT
ATCTTCCTGGCATCTTCCAGGAAATCTCCGCCCCGTTCGTAAGCC
ATTTCCGCTCGCCGCAGTCGAACGACCGAGCGTAGCGAGTCAGTG
AGCGAGGAAGCGGAATATATCCTGTATCACATATTCTGCTGACGC
ACCGGTGCAGCCTTTTTTCTCCTGCCACATGAAGCACTTCACTGA
CACCCTCATCAGTGCCAACATAGTAAGCCAGTATACACTCCGCTA
GCGCTGAGGTCTGCCTCGTGAAGAAGGTGTTGCTGACTCATACCA
GGCCTGAATCGCCCCATCATCCAGCCAGAAAGTGAGGGAGCCACG
GTTGATGAGAGCTTTGTTGTAGGTGGACCAGTTGGTGATTTTGAA
CTTTTGCTTTGCCACGGAACGGTCTGCGTTGTCGGGAAGATGCGT
GATCTGATCCTTCAACTCAGCAAAAGTTCGATTTATTCAACAAAG
CCACGTTGTGTCTCAAAATCTCTGATGTTACATTGCACAAGATAA
AAATATATCATCATGAACAATAAAACTGTCTGCTTACATAAACAG
TAATACAAGGGGTGTTATGAGCCATATTCAACGGGA
α-tubulin RNA from Varroa destructor:
SEQ ID NO: 110
ATGGAGAACATCGCACAGGACTTCGGTAAAAAGTGCCGATTGGGC
TTCGCCATCTACCCGGCTCCGCAGGTTTCCACTGCCGTTGTCGAA
CCATACAACTCGGTTTTGACGACACATGCCACCCTCGAACACGCT
GACTGCGTATTCATGATGGATAATGAGGCGATCTATCAGATCTGT
CGTCGGAATCTTGGAGTTGAACGACCGGCGTATCAAAATCTCAAT
CGACTGATTAGCCAGGCCGTTTCGGCGATAACCGCTTCTCTACGT
TTTTCCGGAGCGTTGAATGTTGACCTCAACGAATTTCAGACGAAT
CTCGTCCCCTACCCGCGAATCCATTTCCCGCTCGTCACTTATGCT
CCGATTATTTCGGCTGAGAAGGCTCATCACGAGCAACATAACGTA
CTGGAA
α-tubulin antisense RNA:
SEQ ID NO: 111
TTCCAGTACGTTATGTTGCTCGTGATGAGCCTTCTCAGCCGAAAT
AATCGGAGCATAAGTGACGAGCGGGAAATGGATTCGCGGGTAGGG
GACGAGATTCGTCTGAAATTCGTTGAGGTCAACATTCAACGCTCC
GGAAAAACGTAGAGAAGCGGTTATCGCCGAAACGGCCTGGCTAAT
CAGTCGATTGAGATTTTGATACGCCGGTCGTTCAACTCCAAGATT
CCGACGACAGATCTGATAGATCGCCTCATTATCCATCATGAATAC
GCAGTCAGCGTGTTCGAGGGTGGCATGTGTCGTCAAAACCGAGTT
GTATGGTTCGACAACGGCAGTGGAAACCTGCGGAGCCGGGTAGAT
GGCGAAGCCCAATCGGCACTTTTTACCGAAGTCCTGTGCGATGTT
CTCCAT

b) Integration of Lambda DE3 Region in cg1121-cg1122 of Corynebacterium glutamicum ATCC 13032 Δcg2273

The T7 RNA polymerase under control of the lacUV5 promoter is expressed from the lambda DE3 phage construct (Moffat er al. (1984) Nucleotide sequence of the gene for bacteriophage T7 RNA polymerase, J Mol Biol 173 265-269). The DE3 fragment was used by Kortmann and co-workers to construct plasmid pK18mobsacB-DE3 (SEQ ID NO: 115) for integration into the intergenic region of cg1121 and cg1122 of C. glutamicum (Kortmann, Kuhl, Klaffl, Bott. 2015. A chromosomally encoded T7 RNA polymerase-dependent gene expression system for Corynebacterium glutamicum_construction and comparative evaluation at the single-cell level. Microbial Biotechnology, 8(2)253-265). Competent cells of the C. glutamicum strain ATCC 13032 Δcg2273 were prepared and transformed by electroporation with pK18mobsacB-DE3 according to Tauch et al., 2002 (Tauch, A., Kirchner, O., Löffler, B., Götker, S., Pühler, A., and Kalinowski, J. Efficient electrotransformation of Corynebacterium diphtheriae with a Mini-Replicon Derived from the Corynebacterium glutamicum plasmid pGA1. Curr. Microbiol. 2002; 45, 362-367). The selection of the transformants was carried out on BHI (brain heart infusion) agar (1%) plates with 25 μg/ml of kanamycin. First and second recombination was conducted as previously described by Niebisch and Bott, 2001 (Niebisch and Bott. Molecular analysis of the cytochrome bc1-aa3 branch of the Corynebacterium glutamicum respiratory chain containing an unusual diheme cytochrome c1. Arch. Microbiol. 2001; 175, 282-294). Resulting clones were verified by colony-PCR using Primers DE3_for (SEQ ID NO: 116) and DE3_rev (SEQ ID NO: 117). The resulting strain is named C. glutamicum ATCC 13032(DE3)_Δcg2273.

pK18mobsacB-DE3:
SEQ ID NO: 115
CTAGCTTCACGCTGCCGCAAGCACTCAGGGCGCAAGGGCTGCTAA
AGGAAGCGGAACACGTAGAAAGCCAGTCCGCAGAAACGGTGCTGA
CCCCGGATGAATGTCAGCTACTGGGCTATCTGGACAAGGGAAAAC
GCAAGCGCAAAGAGAAAGCAGGTAGCTTGCAGTGGGCTTACATGG
CGATAGCTAGACTGGGCGGTTTTATGGACAGCAAGCGAACCGGAA
TTGCCAGCTGGGGCGCCCTCTGGTAAGGTTGGGAAGCCCTGCAAA
GTAAACTGGATGGCTTTCTTGCCGCCAAGGATCTGATGGCGCAGG
GGATCAAGATCTGATCAAGAGACAGGATGAGGATCGTTTCGCATG
ATTGAACAAGATGGATTGCACGCAGGTTCTCCGGCCGCTTGGGTG
GAGAGGCTATTCGGCTATGACTGGGCACAACAGACAATCGGCTGC
TCTGATGCCGCCGTGTTCCGGCTGTCAGCGCAGGGGCGCCCGGTT
CTTTTTGTCAAGACCGACCTGTCCGGTGCCCTGAATGAACTCCAA
GACGAGGCAGCGCGGCTATCGTGGCTGGCCACGACGGGCGTTCCT
TGCGCAGCTGTGCTCGACGTTGTCACTGAAGCGGGAAGGGACTGG
CTGCTATTGGGCGAAGTGCCGGGGCAGGATCTCCTGTCATCTCAC
CTTGCTCCTGCCGAGAAAGTATCCATCATGGCTGATGCAATGCGG
CGGCTGCATACGCTTGATCCGGCTACCTGCCCATTCGACCACCAA
GCGAAACATCGCATCGAGCGAGCACGTACTCGGATGGAAGCCGGT
CTTGTCGATCAGGATGATCTGGACGAAGAGCATCAGGGGCTCGCG
CCAGCCGAACTGTTCGCCAGGCTCAAGGCGCGGATGCCCGACGGC
GAGGATCTCGTCGTGACCCATGGCGATGCCTGCTTGCCGAATATC
ATGGTGGAAAATGGCCGCTTTTCTGGATTCATCGACTGTGGCCGG
CTGGGTGTGGCGGACCGCTATCAGGACATAGCGTTGGCTACCCGT
GATATTGCTGAAGAGCTTGGCGGCGAATGGGCTGACCGCTTCCTC
GTGCTTTACGGTATCGCCGCTCCCGATTCGCAGCGCATCGCCTTC
TATCGCCTTCTTGACGAGTTCTTCTGAGCGGGACTCTGGGGTTCG
CTAGAGGATCGATCCTTTTTAACCCATCACATATACCTGCCGTTC
ACTATTATTTAGTGAAATGAGATATTATGATATTTTCTGAATTGT
GATTAAAAAGGCAACTTTATGCCCATGCAACAGAAACTATAAAAA
ATACAGAGAATGAAAAGAAACAGATAGATTTTTTAGTTCTTTAGG
CCCGTAGTCTGCAAATCCTTTTATGATTTTCTATCAAACAAAAGA
GGAAAATAGACCAGTTGCAATCCAAACGAGAGTCTAATAGAATGA
GGTCGAAAAGTAAATCGCGCGGGTTTGTTACTGATAAAGCAGGCA
AGACCTAAAATGTGTAAAGGGCAAAGTGTATACTTTGGCGTCACC
CCTTACATATTTTAGGTCTTTTTTTATTGTGCGTAACTAACTTGC
CATCTTCAAACAGGAGGGCTGGAAGAAGCAGACCGCTAACACAGT
ACATAAAAAAGGAGACATGAACGATGAACATCAAAAAGTTTGCAA
AACAAGCAACAGTATTAACCTTTACTACCGCACTGCTGGCAGGAG
GCGCAACTCAAGCGTTTGCGAAAGAAACGAACCAAAAGCCATATA
AGGAAACATACGGCATTTCCCATATTACACGCCATGATATGCTGC
AAATCCCTGAACAGCAAAAAAATGAAAAATATCAAGTTTCTGAAT
TTGATTCGTCCACAATTAAAAATATCTCTTCTGCAAAAGGCCTGG
ACGTTTGGGACAGCTGGCCATTACAAAACGCTGACGGCACTGTCG
CAAACTATCACGGCTACCACATCGTCTTTGCATTAGCCGGAGATC
CTAAAAATGCGGATGACACATCGATTTACATGTTCTATCAAAAAG
TCGGCGAAACTTCTATTGACAGCTGGAAAAACGCTGGCCGCGTCT
TTAAAGACAGCGACAAATTCGATGCAAATGATTCTATCCTAAAAG
ACCAAACACAAGAATGGTCAGGTTCAGCCACATTTACATCTGACG
GAAAAATCCGTTTATTCTACACTGATTTCTCCGGTAAACATTACG
GCAAACAAACACTGACAACTGCACAAGTTAACGTATCAGCATCAG
ACAGCTCTTTGAACATCAACGGTGTAGAGGATTATAAATCAATCT
TTGACGGTGACGGAAAAANCGTATCAAAATGTACAGCAGTTCATC
GATGAAGGCAACTACAGCTCAGGCGACAACCATACGCTGAGAGAT
CCTCACTACGTAGAAGATAAAGGCCACAAATACTTAGTATTTGAA
GCAAACACTGGAACTGAAGATGGCTACCAAGGCGAAGAATCTTTA
TTTAACAAAGCATACTATGGCAAAAGCACATCATTCTTCCGTCAA
GAAAGTCAAAAACTTCTGCAAAGCGATAAAAAACGCACGGCTGAG
TTAGCAAACGGCGCTCTCGGTATGATTGAGCTAAACGATGATTAC
ACACTGAAAAAAGTGATGAAACCGCTGATTGCATCTAACACAGTA
ACAGATGAAATTGAACGCGCGAACGTCTTTAAAATGAACGGCAAA
TGGTACCTGTTCACTGACTCCCGCGGATCAAAAATGACGATTGAC
GGCATTACGTCTAACGATATTTACATGCTTGGTTATGTTTCTAAT
TCTTTAACTGGCCCATACAAGCCGCTGAACAAAACTGGCCTTGTG
TTAAAAATGGATCTTGATCCTAACGATGTAACCTTTACTTACTCA
CACTTCGCTGTACCTCAAGCGAAAGGAAACAATGTCGTGATTACA
AGCTATATGACAAACAGAGGATTCTACGCAGACAAACAATCAACG
TTTGCGCCGAGCTTCCTGCTGAACATCAAAGGCAAGAAAACATCT
GTTGTCAAAGACAGCATCCTTGAACAAGGACAATTAACAGTTAAC
AAATAAAAACGCAAAAGAAAATGCCGATGGGTACCGAGCGAAATG
ACCGACCAAGCGACGCCCAACCTGCCATCACGAGATTTCGATTCC
ACCGCCGCCTTCTATGAAAGGTTGGGCTTCGGAATCGTTTTCCGG
GACGCCCTCGCGGACGTGCTCATAGTCCACGACGCCCGTGATTTT
GTAGCCCTGGCCGACGGCCAGCAGGTAGGCCGACAGGCTCATGCC
GGCCGCCGCCGCCTTTTCCTCAATCGCTCTTCGTTCGTCTGGAAG
GCAGTACACCTTGATAGGTGGGCTGCCCTTCCTGGTTGGCTTGGT
TTCATCAGCCATCCGCTTGCCCTCATCTGTTACGCCGGCGGTAGC
CGGCCAGCCTCGCAGAGCAGGATTCCCGTTGAGCACCGCCAGGTG
CGAATAAGGGACAGTGAAGAAGGAACACCCGCTCGCGGGTGGGCC
TACTTCACCTATCCTGCCCCGCTGACGCCGTTGGATACACCAAGG
AAAGTCTACACGAACCCTTTGGCAAAATCCTGTATATCGTGCGAA
AAAGGATGGATATACCGAAAAAATCGCTATAATGACCCCGAAGCA
GGGTTATGCAGCGGAAAAGCGCTGCTTCCCTGCTGTTTTGTGGAA
TATCTACCGACTGGAAACAGGCAAATGCAGGAAATTACTGAACTG
AGGGGACAGGCGAGAGACGATGCCAAAGAGCTCCTGAAAATCTCG
ATAACTCAAAAAATACGCCCGGTAGTGATCTTATTTCATTATGGT
GAAAGTTGGAACCTCTTACGTGCCGATCAACGTCTCATTTTCGCC
AAAAGTTGGCCCAGGGCTTCCCGGTATCAACAGGGACACCAGGAT
TTATTTATTCTGCGAAGTGATCTTCCGTCACAGGTATTTATTCGG
CGCAAAGTGCGTCGGGTGATGCTGCCAACTTACTGATTTAGTGTA
TGATGGTGTTTTTGAGGTGCTCCAGTGGCTTCTGTTTCTATCAGC
TCCTGAAAATCTCGATAACTCAAAAAATACGCCCGGTAGTGATCT
TATTTCATTATGGTGAAAGTTGGAACCTCTTACGTGCCGATCAAC
GTCTCATTTTCGCCAAAAGTTGGCCCAGGGCTTCCCGGTATCAAC
AGGGACACCAGGATTTATTTATTCTGCGAAGTGATCTTCCGTCAC
AGGTATTTATTCGGCGCAAAGTGCGTCGGGTGATGCTGCCAACTT
ACTGATTTAGTGTATGATGGTGTTTTTGAGGTGCTCCAGTGGCTT
CTGTTTCTATCAGGGCTGGATGATCCTCCAGCGCGGGGATCTCAT
GCTGGAGTTCTTCGCCCACCCCAAAAGGATCTAGGTGAAGATCCT
TTTTGATAATCTCATGACCAAAATCCCTTAACGTGAGTTTTCGTT
CCACTGAGCGTCAGACCCCGTAGAAAAGATCAAAGGATCTTCTTG
AGATCCTTTTTTTCTGCGCGTAATCTGCTGCTTGCAAACAAAAAA
ACCACCGCTACCAGCGGTGGTTTGTTTGCCGGATCAAGAGCTACC
AACTCTTTTTCCGAAGGTAACTGGCTTCAGCAGAGCGCAGATACC
AAATACTGTCCTTCTAGTGTAGCCGTAGTTAGGCCACCACTTCAA
GAACTCTGTAGCACCGCCTACATACCTCGCTCTGCTAATCCTGTT
ACCAGTGGCTGCTGCCAGTGGCGATAAGTCGTGTCTTACCGGGTT
GGACTCAAGACGATAGTTACCGGATAAGGCGCAGCGGTCGGGCTG
AACGGGGGGTTCGTGCACACAGCCCAGCTTGGAGCGAACGACCTA
CACCGAACTGAGATACCTACAGCGTGAGCATTGAGAAAGCGCCAC
GCTTCCCGAAGGGAGAAAGGCGGACAGGTATCCGGTAAGCGGCAG
GGTCGGAACAGGAGAGCGCACGAGGGAGCTTCCAGGGGGAAACGC
CTGGTATCTTTATAGTCCTGTCGGGTTTCGCCACCTCTGACTTGA
GCGTCGATTTTTGTGATGCTCGTCAGGGGGGCGGAGCCTATGGAA
AAACGCCAGCAACGCGGCCTTTTTACGGTTCCTGGCCTTTTGCTG
GCCTTTTGCTCACATGTTCTTTCCTGCGTTATCCCCTGATTCTGT
GGATAACCGTATTACCGCCTTTGAGTGAGCTGATACCGCTCGCCG
CAGCCGAACGACCGAGCGCAGCGAGTCAGTGAGCGAGGAAGCGGA
AGAGCGCCCAATACGCAAACCGCCTCTCCCCGCGCGTTGGCCGAT
TCATTAATGCAGCTGGCACGACAGGTTTCCCGACTGGAAAGCGGG
CAGTGAGCGCAACGCAATTAATGTGAGTTAGCTCACTCATTAGGC
ACCCCAGGCTTTACACTTTATGCTTCCGGCTCGTATGTTGTGTGG
AATTGTGAGCGGATAACAATTTCACACAGGAAACAGCTATGACCA
TGATTACGAATTGGCTCCGTTGTTCACGTCTACTACGACGGCGAC
GAGAACGACAAGGAAACCTTCCTCATCGGTACCCGTGCTGGCGCT
TCCGAGAACCCAGATCTTGAGACCTACTCTGAGCAGTCCCCACTC
GGCGCTGCAATTCTCGGAGCTCAGGAAGGCGACACCCGTCAGTAC
ACCGCTCCAAATGGTTCCGTTATCTCCGTAACTGTTGTTTCTGCA
GAACCATACAACTCAGCAAAAGCCGCGACACTCCGCGGCAAAAAC
TAACCAAGGATTTAAAAGTCTTCAAAATGACAACTCTTTCACGTA
AGTTCTTCGTTTCTGCTACCACAGCCCTGGCGGCAGTCGCACTGG
TTGCGTGTTCCCCTAATGAGATTGATTCTGAACTGAAGGTGCCAA
CGGCAACTGGCGTTTCTTTACCTTCGAAGAACGTTTCCGCGACCT
CAACTGCTACTACAGATGAGGATGCGCCTGGCTACATTGATTGCG
TAGCCGCACCAACTCAGCAACCTGCTGAAATCTCACTAAACTGTG
CAATGGATATTGATCGGCTCACGGATATTTCTTGGAGCGAATGGG
ATACTGATTCCGCAACTGGAACCGGTACCCGCATCGTAACCGCTG
CAAATGGTCAAGAGACCGAAACCGAAGATATTGAGGTGAAGCTTT
CCTTCCCCACCGAGTCTTCCCAAGGCCTAGTGTTCACTCAGGTCA
CCGTCGATGGACAGGTTCTCTTCCTCTAATCCTCCATAATTAGAG
AGCGTAAGGCCCCTACTTCCTGTTTTAGGAAATAGGGGCCTTTTG
TTGTCTTCTCCTGGAGGCTATTTAAGAAGTTTAAATTGTGTCCAT
GAGTTCGCTCGAGAACTGCGCAACTCGTGAAAGGTAGGCGGATCC
AGATCCCGGACACCATCGAATGGCGCAAAACCTTTCGCGGTATGG
CATGATAGCGCCCGGAAGAGAGTCAATTCAGGGTGGTGAATGTGA
AACCAGTAACGTTATACGATGTCGCAGAGTATGCCGGTGTCTCTT
ATCAGACCGTTTCCCGCGTGGTGAACCAGGCCAGCCACGTTTCTG
CGAAAACGCGGGAAAAAGTGGAAGCGGCGATGGCGGAGCTGAATT
ACATTCCCAACCGCGTGGCACAACAACTGGCGGGCAAACAGTCGT
TGCTGATTGGCGTTGCCACCTCCAGTCTGGCCCTGCACGCGCCGT
CGCAAATTGTCGCGGCGATTAAATCTCGCGCCGATCAACTGGGTG
CCAGCGTGGTGGTGTCGATGGTAGAACGAAGCGGCGTCGAAGCCT
GTAAAGCGGCGGTGCACAATCTTCTCGCGCAACGCGTCAGTGGGC
TGATCATTAACTATCCGCTGGATGACCAGGATGCCATTGCTGTGG
AAGCTGCCTGCACTAATGTTCCGGCGTTATTTCTTGATGTCTCTG
ACCAGACACCCATCAACAGTATTATTTTCTCCCATGAAGACGGTA
CGCGACTGGGCGTGGAGCATCTGGTCGCATTGGGTCACCAGCAAA
TCGCGCTGTTAGCGGGCCCATTAAGTTCTGTCTCGGCGCGTCTGC
GTCTGGCTGGCTGGCATAAATATCTCACTCGCAATCAAATTCAGC
CGATAGCGGAACGGGAAGGCGACTGGAGTGCCATGTCCGGTTTTC
AACAAACCATGCAAATGCTGAATGAGGGCATCGTTCCCACTGCGA
TGCTGGTTGCCAACGATCAGATGGCGCTGGGCGCAATGCGCGCCA
TTACCGAGTCCGGGCTGCGCGTTGGTGCGGATATCTCGGTAGTGG
GATACGACGATACCGAAGACAGCTCATGTTATATCCCGCCGTTAA
CCACCATCAAACAGGATTTTCGCCTGCTGGGGCAAACCAGCGTGG
ACCGCTTGCTGCAACTCTCTCAGGGCCAGGCGGTGAAGGGCAATC
AGCTGTTGCCCGTCTCACTGGTGAAAAGAAAAACCACCCTGGCGC
CCAATACGCAAACCGCCTCTCCCCGCGCGTTGGCCGATTCATTAA
TGCAGCTGGCACGACAGGTTTCCCGACTGGAAAGCGGGCAGTGAG
CGCAACGCAATTAATGTAAGTTAGCTCACTCATTAGGCACCCCAG
GCTTTACACTTTATGCTTCCGGCTCGTATAATGTGTGGAATTGTG
AGCGGATAACAATTTCACACAGGAAACAGCTATGACCATGATTAC
GGATTCACTGGCCGTCGTTTTACAACGTCGTGACTGGGAAAACCC
TGGCGTTACCCAACTTAATCGCCTTGCAGCACATCCCCCTTTCGC
CAGCTGGCGTAATAGCGAAGAGGCCCGCACCGATCGCCCTTCCCA
ACAGTTGCGCAGCCTGAATGGCGAATGGCGCTTTGCCTGGTTTCC
GGCACCAGAAGCGGTGCCGGAAAGCTGGCTGGAGTGCGATCTTCC
TGAGGCCGATACTGTCGTCGTCCCCTCAAACTGGCAGATGCACGG
TTACGATGCGCCCATCTACACCAACGTGACCTATCCCATTACGGT
CAATCCGCCGTTTGTTCCCACGGAGAATCCGACGGGTTGTTACTC
GCTCACATTTAATGTTGATGAAAGCTGGCTACAGGAAGGCCAGAC
GCGAATTATTTTTGATGGCGTCGGGATCTGATCCGGATTTACTAA
CTGGAAGAGGCACTAAATGAACACGATTAACATCGCTAAGAACGA
CTTCTCTGACATCGAACTGGCTGCTATCCCGTTCAACACTCTGGC
TGACCATTACGGTGAGCGTTTAGCTCGCGAACAGTTGGCCCTTGA
GCATGAGTCTTACGAGATGGGTGAAGCACGCTTCCGCAAGATGTT
TGAGCGTCAACTTAAAGCTGGTGAGGTTGCGGATAACGCTGCCGC
CAAGCCTCTCATCACTACCCTACTCCCTAAGATGATTGCACGCAT
CAACGACTGGTTTGAGGAAGTGAAAGCTAAGCGCGGCAAGCGCCC
GACAGCCTTCCAGTTCCTGCAAGAAATCAAGCCGGAAGCCGTAGC
GTACATCACCATTAAGACCACTCTGGCTTGCCTAACCAGTGCTGA
CAATACAACCGTTCAGGCTGTAGCAAGCGCAATCGGTCGGGCCAT
TGAGGACGAGGCTCGCTTCGGTCGTATCCGTGACCTTGAAGCTAA
GCACTTCAAGAAAAACGTTGAGGAACAACTCAACAAGCGCGTAGG
GCACGTCTACAAGAAAGCATTTATGCAAGTTGTCGAGGCTGACAT
GCTCTCTAAGGGTCTACTCGGTGGCGAGGCGTGGTCTTCGTGGCA
TAAGGAAGACTCTATTCATGTAGGAGTACGCTGCATCGAGATGCT
CATTGAGTCAACCGGAATGGTTAGCTTACACCGCCAAAATGCTGG
CGTAGTAGGTCAAGACTCTGAGACTATCGAACTCGCACCTGAATA
CGCTGAGGCTATCGCAACCCGTGCAGGTGCGCTGGCTGGCATCTC
TCCGATGTTCCAACCTTGCGTAGTTCCTCCTAAGCCGTGGACTGG
CATTACTGGTGGTGGCTATTGGGCTAACGGTCGTCGTCCTCTGGC
GCTGGTGCGTACTCACAGTAAGAAAGCACTGATGCGCTACGAAGA
CGTTTACATGCCTGAGGTGTACAAAGCGATTAACATTGCGCAAAA
CACCGCATGGAAAATCAACAAGAAAGTCCTAGCGGTCGCCAACGT
AATCACCAAGTGGAAGCATTGTCCGGTCGAGGACATCCCTGCGAT
TGAGCGTGAAGAACTCCCGATGAAACCGGAAGACATCGACATGAA
TCCTGAGGCTCTCACCGCGTGGAAACGTGCTGCCGCTGCTGTGTA
CCGCAAGGACAAGGCTCGCAAGTCTCGCCGTATCAGCCTTGAGTT
CATGCTTGAGCAAGCCAATAAGTTTGCTAACCATAAGGCCATCTG
GTTCCCTTACAACATGGACTGGCGCGGTCGTGTTTACGCTGTGTC
AATGTTCAACCCGCAAGGTAACGATATGACCAAAGGACTGCTTAC
GCTGGCGAAAGGTAAACCAATCGGTAAGGAAGGTTACTACTGGCT
GAAAATCCACGGTGCAAACTGTGCGGGTGTCGATAAGGTTCCGTT
CCCTGAGCGCATCAAGTTCATTGAGGAAAACCACGAGAACATCAT
GGCTTGCGCTAAGTCTCCACTGGAGAACACTTGGTGGGCTGAGCA
AGATTCTCCGTTCTGCTTCCTTGCGTTCTGCTTTGAGTACGCTGG
GGTACAGCACCACGGCCTGAGCTATAACTGCTCCCTTCCGCTGGC
GTTTGACGGGTCTTGCTCTGGCATCCAGCACTTCTCCGCGATGCT
CCGAGATGAGGTAGGTGGTCGCGCGGTTAACTTGCTTCCTAGTGA
AACCGTTCAGGACATCTACGGGATTGTTGCTAAGAAAGTCAACGA
GATTCTACAAGCAGACGCAATCAATGGGACCGATAACGAAGTAGT
TACCGTGACCGATGAGAACACTGGTGAAATCTCTGAGAAAGTCAA
GCTGGGCACTAAGGCACTGGCTGGTCAATGGCTGGCTTACGGTGT
TACTCGCAGTGTGACTAAGCGTTCAGTCATGACGCTGGCTTACGG
GTCCAAAGAGTTCGGCTTCCGTCAACAAGTGCTGGAAGATACCAT
TCAGCCAGCTATTGATTCCGGCAAGGGTCTGATGTTCACTCAGCC
GAATCAGGCTGCTGGATACATGGCTAAGCTGATTTGGGAATCTGT
GAGCGTGACGGTGGTAGCTGCGGTTGAAGCAATGAACTGGCTTAA
GTCTGCTGCTAAGCTGCTGGCTGCTGAGGTCAAAGATAAGAAGAC
TGGAGAGATTCTTCGCAAGCGTTGCGCTGTGCATTGGGTAACTCC
TGATGGTTTCCCTGTGTGGCAGGAATACAAGAAGCCTATTCAGAC
GCGCTTGAACCTGATGTTCCTCGGTCAGTTCCGCTTACAGCCTAC
CATTAACACCAACAAAGATAGCGAGATTGATGCACACAAACAGGA
GTCTGGTATCGCTCCTAACTTTGTACACAGCCAAGACGGTAGCCA
CCTTCGTAAGACTGTAGTGTGGGCACACGAGAAGTACGGAATCGA
ATCTTTTGCACTGATTCACGACTCCTTCGGTACCATTCCGGCTGA
CGCTGCGAACCTGTTCAAAGCAGTGCGCGAAACTATGGTTGACAC
ATATGAGTCTTGTGATGTACTGGCTGATTTCTACGACCAGTTCGC
TGACCAGTTGCACGAGTCTCAATTGGACAAAATGCCAGCACTTCC
GGCTAAAGGTAACTTGAACCTCCGTGACATCTTAGAGTCGGACTT
CGCGTTCGCGTAACGAATTCGCGTATGGCAATGACAGTTTGAGAC
GGCCACAGGCGATTCTGAGAAGCCATTTTCTTTGGGCGCCGTGGC
AGTTTTTATTGGGTCCCACCGCCGAACTGCATATTCGAACCAAGG
AGCCTCAAAAATCGAGCTCGCTTTGGTCTCAAACGCACATTTATC
GCGCGTTGAAGTGTGCGTTTGAGACCAAAGAGCCCTCCACAACGC
ACGTCTTTGGTTTGGATATGACAGGTGCCCAAGAACTCACCCCGC
CCCATGCTCACAGAGCCCCCATCAGAAGCCAAAAGACCCCTTCCC
TGCCCAAGAAGAACAGGATGAAGGGGTCTTGTGCTGCGTAAACTA
GCGGTTTTGGAAGTAGCTAAGCAGACGTAGGATTTCGGTGTAGAG
CCAGACCAAGGTCACTGCAAGACCAAGCGCAACGCCCCATGCCAT
CTTGGAAGGTGCACCTTCGCGGACGAGGCGGTCAGCTGCATCGAA
GTCGGAGAGGAAGCTGAATGCTGCCAGGCCGATGCAGAAGAGGGA
GAAGATAATCGCGATGATTCCACCGTCACGCAGTGGGCTTGCGCC
ACCAGTGAACAGTGCCCATACAACGTTGCCCAGGACAAGAACCAG
GACGCCAACCATCATGCCGGTGAGGATGCGGTTGAACTTAGGAGT
GACCTTGATAGCGCCAGTCTTGTATACAAACAGCATGCCAATGAA
TACACCGATGGTGCCAAGGACTGCCTGGCCAATGAGGCCACCTGC
GTTGGCGTTACCAACTGTGAAGCCGGACAGCAGAAGGGAAATTCC
GCCGACGAAGAGGCCTTCGAATACTGCGTAAATCAAAGTGACTGC
CGCAGAT
DE3_for
SEQ ID NO: 116
TTTGGCGTGTGGTTGGTTAG
DE3_rev
SEQ ID NO: 117
TCTCTGAGCTGCTGGCCAAC

c) Transformation of C. glutamicum ATCC 13032(DE3) Δcg2273 with pJC1_dsRNA_PT7-αTubulin-F30::broccoli or pJC1_dsRNA_PT7-CYP3-F30::broccoli

Competent cells of the C. glutamicum strain ATCC 13032(DE3)_Δcg2273 were prepared and transformed with pJC1 or pJC1_dsRNA_PT7-αtubulin-F30::broccoli according to Tauch et al., 2002 (Tauch, A., Kirchner, O., Löffler, B., Götker, S., Pühler, A., and Kalinowski, J. Efficient Electrotransformation of Corynebacterium diphtheriae with a Mini-Replicon Derived from the Corynebacterium glutamicum Plasmid pGA1. Curr. Microbiol. 2002; 45, 362-367). The selection of the transformants was carried out on BHI agar (1%) plates with 25 μg/ml of kanamycin (Menkel, E., Thierbach, G., Eggeling, L., and Sahm, H. Influence of increased aspartate availability on lysine formation by a recombinant strain of Corynebacterium glutamicum and utilization of fumarate. Appl. Environ. Microbiol. 1989; 55, 684-688). Clones thus obtained were named C. glutamicum ATCC 13032(DE3)_Δcg2273 pJC1 or C. glutamicum ATCC 13032(DE3)_Δcg2273 pJC1_dsRNA_PT7-αtubulin-F30::broccoli, depending on which plasmid was used for transformation.

### d) Cultivation of Cells for Phenotype Validation Using Fluorescence Activated Cell Sorting (FACS)

The produced strains C. glutamicum ATCC 13032(DE3)_Δcg2273 pJC1 and C. glutamicum ATCC 13032(DE3)_Δcg2273 pJC1_dsRNA_PT7-αtubulin-F30::broccoli were streaked on BHI agar plates containing 25 μg/mL kanamycin and cultivated at 30° C. Grown cells were resuspended in CGIII cultivation medium containing 25 μg/mL kanamycin and the OD₆₀₀ was adjusted to 0.75 in a tube containing 2 mL cultivation medium with antibiotic. Cells were incubated at 30° C. and 120 rpm and induced by 1.5 mM IPTG after six hours of cultivation. Afterwards, incubation was continued for six hours. Subsequently, cells were diluted to an OD₆₀₀ of 0.6 using PBS with a final concentration of 500 μM DFHBI. Cells were analyzed using an AriaIII High-speed cell sorter as already described in example 1d). DFHBI-stained DFHBI-stained C. glutamicum ATCC 13032(DE3)_Δcg2273 pJC1_dsRNA_PT7-αtubulin-F30::broccoli showed an about five-fold increased fluorescent output compared to unstained cells, while DFHBI-stained C. glutamicum ATCC 13032(DE3)_Δcg2273 pJC1 showed 1,2-fold increased fluorescent output compared to unstained cells (cf. FIG. 6).

e) Extraction of the RNA of Interest from the Cells Isolated in d)

Using the culture broths analyzed in c), 1.38×10⁹ cells from the cultures were used for RNA extraction with the Monarch total RNA kit (New England Biolabs, Ipswich, MA, USA) as already described in example 1.

### f) Verification of the Formation of a Double-Stranded RNA Product by RNaseIII and RNaseA Digestion

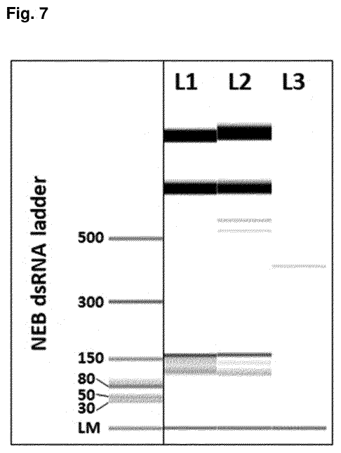

Verification of the formation of a double-stranded RNA product by RNase A digestion. A total of 2 μg of RNA isolated from the cultivation of C. glutamicum ATCC 13032(DE3)_Δcg2273 pJC1_dsRNA_PT7-αtubulin-F30::broccoli was treated with 50 ng RNase A for one hour at room temperature, in the presence of 300 mM NaCl, according to the manufacturer's recommendation (AppliChem GmbH, Darmstadt, Germany), to remove mRNA, rRNA and to remove the single-stranded part of the target RNA (cf. FIG. 5). Following the RNase A treatment, the reaction was purified using a T2030 Monarch RNA Cleanup Kit (New England Biolabs, Ipswich, MA, USA). A dsRNA Ladder (New England Biolabs, Ipswich, MA, USA), the isolated total RNA extracted in e) and the RNase A-treated RNA were analyzed using an Agilent Fragment Analyzer equipped with a DNF-471 RNA kit (Agilent Technologies, Santa Clara, CA, USA). Two bands, only visible in the total RNA extraction from C. glutamicum ATCC 13032(DE3)_Δcg2273 pJC1_dsRNA_PT7-αtubulin-F30::broccoli, resemble the calculated fragment size of the target RNA fragment (522 nt, lower fragment) and the calculated fragment size of the target RNA including the F1 terminator at the end of the αtubulin asRNA strand (575 nt, bigger fragment) (cf. FIG. 7). After the RNase A reaction, one fragment with the exact size of the αtubulin dsRNA was visible, all mRNA and rRNA was degraded.

This experiment shows the successful linking of a hitherto unsuspicious phenotype (dsRNA production) with a fluorescence output. In this experiment, the produced dsRNA has a length of 411 nucleotides and is transcribed from a vector. In accordance with the procedure shown in example 1, the optimization of the fermentative production of dsRNA, is therefore possible using the invention.

### Example 7

a) Construction of the Vectors pJC1-PT7-egfp-broccoli-TT7 and pJC1-PT7-Luc2-broccoli-TT7

The construction of the plasmid was achieved by means of chemical synthesis of synthetic DNA-fragments (SEQ ID NO: 118 for PT7-egfp-broccoli-TT7 and SEQ ID NO: 119 for PT7-luc2-broccoli-TT7), and their insertion into restriction sites BamHI and EcoRV of pJC1 resulting in plasmids pJC1_PT7-egfp-broccoli-TT7 (SEQ ID NO: 120) and pJC1-PT7-luc2-broccoli-TT7 (SEQ ID NO: 121) (ordered from Twist Bioscience, South San Francisco, USA). SEQ ID NO: 118 contained the T7 promoter (SEQ ID NO: 96), a gene egfp encoding an enhanced green fluorescent protein (modified from Aequorea victoria) (SEQ ID NO: 112), the F30 scaffold with a broccoli aptamer in the insertion site (SEQ ID NO: 69) and the T7 terminator (SEQ ID NO: 97). SEQ ID NO: 119 contained the T7 promoter (SEQ ID NO: 96), the gene luc2 encoding the luciferase of Photinus pyralis (SEQ ID NO: 82), the F30 scaffold with a broccoli aptamer (SEQ ID NO: 69) in the insertion site and the T7 terminator (SEQ ID NO: 97).

PT7-egfp-broccoli-TT7 (SEQ ID NO: 96,
112, 69 and 97 combined):
SEQ ID NO: 118
TAATACGACTCACTATAGGGAGACCCAAGCTGGCTAGCGTTTAAA
CTTAAGCTTCGAATTCTGCAGTCGACGGTACCGCGGGCCCGGGAT
CCACCGGTCGCCACCATGGTGAGCAAGGGCGAGGAGCTGTTCACC
GGGGTGGTGCCCATCCTGGTCGAGCTGGACGGCGACGTAAACGGC
CACAAGTTCAGCGTGTCCGGCGAGGGCGAGGGCGATGCCACCTAC
GGCAAGCTGACCCTGAAGTTCATCTGCACCACCGGCAAGCTGCCC
GTGCCCTGGCCCACCCTCGTGACCACCCTGACCTACGGCGTGCAG
TGCTTCAGCCGCTACCCCGACCACATGAAGCAGCACGACTTCTTC
AAGTCCGCCATGCCCGAAGGCTACGTCCAGGAGCGCACCATCTTC
TTCAAGGACGACGGCAACTACAAGACCCGCGCCGAGGTGAAGTTC
GAGGGCGACACCCTGGTGAACCGCATCGAGCTGAAGGGCATCGAC
TTCAAGGAGGACGGCAACATCCTGGGGCACAAGCTGGAGTACAAC
TACAACAGCCACAACGTCTATATCATGGCCGACAAGCAGAAGAAC
GGCATCAAGGTGAACTTCAAGATCCGCCACAACATCGAGGACGGC
AGCGTGCAGCTCGCCGACCACTACCAGCAGAACACCCCCATCGGC
GACGGCCCCGTGCTGCTGCCCGACAACCACTACCTGAGCACCCAG
TCCGCCCTGAGCAAAGACCCCAACGAGAAGCGCGATCACATGGTC
CTGCTGGAGTTCGTGACCGCCGCCGGGATCACTCTCGGCATGGAC
GAGCTGTACAAGTAATACGTATTGCCATGTGTATGTGGGAGACGG
TCGGGTCCAGATATTCGTATCTGTCGAGTAGAGTGTGGGCTCCCA
CATACTCTGATGATCCTTCGGGATCATTCATGGCAAGTCGACCTA
GCATAACCCCTTGGGGCCTCTAAACGGGTCTTGAGGGGTTTTTTG
PT7-luc2-broccoli-TT7 (SEQ ID NO: 96,
82, 69 and 97 combined):
SEQ ID NO: 119
TAATACGACTCACTATAGGGAGACCCAAGCTGGCTAGCGTTTAAA
CTTAAGCTTGGCAATCCGGTACTGTTGGTAAAGCCACCATGGAAG
ATGCCAAAAACATTAAGAAGGGCCCAGCGCCATTCTACCCACTCG
AAGACGGGACCGCCGGCGAGCAGCTGCACAAAGCCATGAAGCGCT
ACGCCCTGGTGCCCGGCACCATCGCCTTTACCGACGCACATATCG
AGGTGGACATTACCTACGCCGAGTACTTCGAGATGAGCGTTCGGC
TGGCAGAAGCTATGAAGCGCTATGGGCTGAATACAAACCATCGGA
TCGTGGTGTGCAGCGAGAATAGCTTGCAGTTCTTCATGCCCGTGT
TGGGTGCCCTGTTCATCGGTGTGGCTGTGGCCCCAGCTAACGACA
TCTACAACGAGCGCGAGCTGCTGAACAGCATGGGCATCAGCCAGC
CCACCGTCGTATTCGTGAGCAAGAAAGGGCTGCAAAAGATCCTCA
ACGTGCAAAAGAAGCTACCGATCATACAAAAGATCATCATCATGG
ATAGCAAGACCGACTACCAGGGCTTCCAAAGCATGTACACCTTCG
TGACTTCCCATTTGCCACCCGGCTTCAACGAGTACGACTTCGTGC
CCGAGAGCTTCGACCGGGACAAAACCATCGCCCTGATCATGAACA
GTAGTGGCAGTACCGGATTGCCCAAGGGCGTAGCCCTACCGCACC
GCACCGCTTGTGTCCGATTCAGTCATGCCCGCGACCCCATCTTCG
GCAACCAGATCATCCCCGACACCGCTATCCTCAGCGTGGTGCCAT
TTCACCACGGCTTCGGCATGTTCACCACGCTGGGCTACTTGATCT
GCGGCTTTCGGGTCGTGCTCATGTACCGCTTCGAGGAGGAGCTAT
TCTTGCGCAGCTTGCAAGACTATAAGATTCAATCTGCCCTGCTGG
TGCCCACACTATTTAGCTTCTTCGCTAAGAGCACTCTCATCGACA
AGTACGACCTAAGCAACTTGCACGAGATCGCCAGCGGCGGGGCGC
CGCTCAGCAAGGAGGTAGGTGAGGCCGTGGCCAAACGCTTCCACC
TACCAGGCATCCGCCAGGGCTACGGCCTGACAGAAACAACCAGCG
CCATTCTGATCACCCCCGAAGGGGACGACAAGCCTGGCGCAGTAG
GCAAGGTGGTGCCCTTCTTCGAGGCTAAGGTGGTGGACTTGGACA
CCGGTAAGACACTGGGTGTGAACCAGCGCGGCGAGCTGTGCGTCC
GTGGCCCCATGATCATGAGCGGCTACGTTAACAACCCCGAGGCTA
CAAACGCTCTCATCGACAAGGACGGCTGGCTGCACAGCGGCGACA
TCGCCTACTGGGACGAGGACGAGCACTTCTTCATCGTGGACCGGC
TGAAGAGCCTGATCAAATACAAGGGCTACCAGGTAGCCCCAGCCG
AACTGGAGAGCATCCTGCTGCAACACCCCAACATCTTCGACGCCG
GGGTCGCCGGCCTGCCCGACGACGATGCCGGCGAGCTGCCCGCCG
CAGTCGTCGTGCTGGAACACGGTAAAACCATGACCGAGAAGGAGA
TCGTGGACTATGTGGCCAGCCAGGTTACAACCGCCAAGAAGCTGC
GCGGTGGTGTTGTGTTCGTGGACGAGGTGCCTAAAGGACTGACCG
GCAAGTTGGACGCCCGCAAGATCCGCGAGATTCTCATTAAGGCCA
AGAAGGGCGGCAAGATCGCCGTGTAATAATTCTAGATACGTATTG
CCATGTGTATGTGGGAGACGGTCGGGTCCAGATATTCGTATCTGT
CGAGTAGAGTGTGGGCTCCCACATACTCTGATGATCCTTCGGGAT
CATTCATGGCAAGTCGACCTAGCATAACCCCTTGGGGCCTCTAAA
CGGGTCTTGAGGGGTTTTTTG
pJC1-PT7-egfp-broccoli-TT7:
SEQ ID NO: 120
CCGAGAAGTTTTTTACAAAAGGCAAAAACTTTTTCGGGATCGACA
GAAATAAAACGATCGACGGTACGCAACAAAAAAGCGTCAGGATCG
CCGTAGAGCGATTGAAGACCGTCAACCAAAGGGGAAGCCTCCAAT
CGACGCGACGCGCGCTCTACGGCGATCCTGACGCAGATTTTTAGC
TATCTGTCGCAGCGCCCTCAGGGACAAGCCACCCGCACAACGTCG
CGAGGGCGATCAGCGACGCCGCAGGGTAGTACTCAGCTGTAATAC
GACTCACTATAGGGAGACCCAAGCTGGCTAGCGTTTAAACTTAAG
CTTCGAATTCTGCAGTCGACGGTACCGCGGGCCCGGGATCCACCG
GTCGCCACCATGGTGAGCAAGGGCGAGGAGCTGTTCACCGGGGTG
GTGCCCATCCTGGTCGAGCTGGACGGCGACGTAAACGGCCACAAG
TTCAGCGTGTCCGGCGAGGGCGAGGGCGATGCCACCTACGGCAAG
CTGACCCTGAAGTTCATCTGCACCACCGGCAAGCTGCCCGTGCCC
TGGCCCACCCTCGTGACCACCCTGACCTACGGCGTGCAGTGCTTC
AGCCGCTACCCCGACCACATGAAGCAGCACGACTTCTTCAAGTCC
GCCATGCCCGAAGGCTACGTCCAGGAGCGCACCATCTTCTTCAAG
GACGACGGCAACTACAAGACCCGCGCCGAGGTGAAGTTCGAGGGC
GACACCCTGGTGAACCGCATCGAGCTGAAGGGCATCGACTTCAAG
GAGGACGGCAACATCCTGGGGCACAAGCTGGAGTACAACTACAAC
AGCCACAACGTCTATATCATGGCCGACAAGCAGAAGAACGGCATC
AAGGTGAACTTCAAGATCCGCCACAACATCGAGGACGGCAGCGTG
CAGCTCGCCGACCACTACCAGCAGAACACCCCCATCGGCGACGGC
CCCGTGCTGCTGCCCGACAACCACTACCTGAGCACCCAGTCCGCC
CTGAGCAAAGACCCCAACGAGAAGCGCGATCACATGGTCCTGCTG
GAGTTCGTGACCGCCGCCGGGATCACTCTCGGCATGGACGAGCTG
TACAAGTAATACGTATTGCCATGTGTATGTGGGAGACGGTCGGGT
CCAGATATTCGTATCTGTCGAGTAGAGTGTGGGCTCCCACATACT
CTGATGATCCTTCGGGATCATTCATGGCAAGTCGACCTAGCATAA
CCCCTTGGGGCCTCTAAACGGGTCTTGAGGGGTTTTTTGCTGAAA
GATATCATCCAATGGCAACAACGTTGCGCAAACTATTAACTGGCG
AACTACTTACTCTAGCTTCCCGGCAACAATTAATAGACTGGATGG
AGGCGGATAAAGTTGCAGGACCACTTCTGCGCTCGGCCCTTCCGG
CTGGCTGGTTTATTGCTGATAAATCTGGAGCCGGTGAGCGTGGGT
CTCGCGGTATCATTGCAGCACTGGGGCCAGATGGTAAGCCCTCCC
GTATCGTAGTTATCTACACGACGGGGAGTCAGGCAACTATGGATG
AACGAAATAGACAGATCGCTGAGATAGGTGCCTCACTGATTAAGC
ATTGGTAACTGTCAGACCAAGTTTACTCATATATACTTTAGATTG
ATTTAAAACTTCATTTTTAATTTAAAAGGATCTAGGTGAAGATCC
TTTTTGATAATCTCATGACCAAAATCCCTTAACGTGAGTTTTCGT
TCCACTGAGCGTCAGACCCCTTAATAAGATGATCTTCTTGAGATC
GTTTTGGTCTGCGCGTAATCTCTTGCTCTGAAAACGAAAAAACCG
CCTTGCAGGGCGGTTTTTCGAAGGTTCTCTGAGCTACCAACTCTT
TGAACCGAGGTAACTGGCTTGGAGGAGCGCAGTCACCAAAACTTG
TCCTTTCAGTTTAGCCTTAACCGGCGCATGACTTCAAGACTAACT
CCTCTAAATCAATTACCAGTGGCTGCTGCCAGTGGTGCTTTTGCA
TGTCTTTCCGGGTTGGACTCAAGACGATAGTTACCGGATAAGGCG
CAGCGGTCGGACTGAACGGGGGGTTCGTGCATACAGTCCAGCTTG
GAGCGAACTGCCTACCCGGAACTGAGTGTCAGGCGTGGAATGAGA
CAAACGCGGCCATAACAGCGGAATGACACCGGTAAACCGAAAGGC
AGGAACAGGAGAGCGCACGAGGGAGCCGCCAGGGGGAAACGCCTG
GTATCTTTATAGTCCTGTCGGGTTTCGCCACCACTGATTTGAGCG
TCAGATTTCGTGATGCTTGTCAGGGGGGCGGAGCCTATGGAAAAA
CGGCTTTGCCGCGGCCCTCTCACTTCCCTGTTAAGTATCTTCCTG
GCATCTTCCAGGAAATCTCCGCCCCGTTCGTAAGCCATTTCCGCT
CGCCGCAGTCGAACGACCGAGCGTAGCGAGTCAGTGAGCGAGGAA
GCGGAATATATCCTGTATCACATATTCTGCTGACGCACCGGTGCA
GCCTTTTTTCTCCTGCCACATGAAGCACTTCACTGACACCCTCAT
CAGTGCCAACATAGTAAGCCAGTATACACTCCGCTAGCGCTGAGG
TCTGCCTCGTGAAGAAGGTGTTGCTGACTCATACCAGGCCTGAAT
CGCCCCATCATCCAGCCAGAAAGTGAGGGAGCCACGGTTGATGAG
AGCTTTGTTGTAGGTGGACCAGTTGGTGATTTTGAACTTTTGCTT
TGCCACGGAACGGTCTGCGTTGTCGGGAAGATGCGTGATCTGATC
CTTCAACTCAGCAAAAGTTCGATTTATTCAACAAAGCCACGTTGT
GTCTCAAAATCTCTGATGTTACATTGCACAAGATAAAAATATATC
ATCATGAACAATAAAACTGTCTGCTTACATAAACAGTAATACAAG
GGGTGTTATGAGCCATATTCAACGGGAAACGTCTTGCTCGAGGCC
GCGATTAAATTCCAACATGGATGCTGATTTATATGGGTATAAATG
GGCTCGCGATAATGTCGGGCAATCAGGTGCGACAATCTATCGATT
GTATGGGAAGCCCGATGCGCCAGAGTTGTTTCTGAAACATGGCAA
AGGTAGCGTTGCCAATGATGTTACAGATGAGATGGTCAGACTAAA
CTGGCTGACGGAATTTATGCCTCTTCCGACCATCAAGCATTTTAT
CCGTACTCCTGATGATGCATGGTTACTCACCACTGCGATCCCCGG
GAAAACAGCATTCCAGGTATTAGAAGAATATCCTGATTCAGGTGA
AAATATTGTTGATGCGCTGGCAGTGTTCCTGCGCCGGTTGCATTC
GATTCCTGTTTGTAATTGTCCTTTTAACAGCGATCGCGTATTTCG
TCTCGCTCAGGCGCAATCACGAATGAATAACGGTTTGGTTGATGC
GAGTGATTTTGATGACGAGCGTAATGGCTGGCCTGTTGAACAAGT
CTGGAAAGAAATGCATAAGCTTTTGCCATTCTCACCGGATTCAGT
CGTCACTCATGGTGATTTCTCACTTGATAACCTTATTTTTGACGA
GGGGAAATTAATAGGTTGTATTGATGTTGGACGAGTCGGAATCGC
AGACCGATACCAGGATCTTGCCATCCTATGGAACTGCCTCGGTGA
GTTTTCTCCTTCATTACAGAAACGGCTTTTTCAAAAATATGGTAT
TGATAATCCTGATATGAATAAATTGCAGTTTCATTTGATGCTCGA
TGAGTTTTTCTAATCAGAATTGGTTAATTGGTTGTAACACTGGCA
GAGCATTACGCTGACTTGACGGGACGGCGGCTTTGTTGAATAAAT
CGAACTTTTGCTGAGTTGAAGGATCAGATCACGCATCTTCCCGAC
AACGCAGACCGTTCCGTGGCAAAGCAAAAGTTCAAAATCACCAAC
TGGTCCACCTACAACAAAGCTCTCATCAACCGTGGCTCCCTCACT
TTCTGGCTGGATGATGGGGCGATTCAGGCCTGGTATGAGTCAGCA
ACACCTTCTTCACGAGGCAGACCTCAGCGCTCAAAGATGCAGGGG
TAAAAGCTAACCGCATCTTTACCGACAAGGCATCCGGCAGTTCAA
CAGATCGGGAAGGGCTGGATTTGCTGAGGATGAAGGTGGAGGAAG
GTGATGTCATTCTGGTGAAGAAGCTCGACCGTCTTGGCCGCGACA
CCGCCGACATGATCCAACTGATAAAAGAGTTTGATGCTCAGGGTG
TAGCGGTTCGGTTTATTGACGACGGGATCAGTACCGACGGTGATA
TGGGGCAAATGGTGGTCACCATCCTGTCGGCTGTGGCACAGGCTG
AACGCCGGAGGATCAAGTCGGTCAAGCCAAGCGCAACCAGCGGCA
CCGCCGCGAGCAACGTCGCAAGGGCGATCAGGGGACGATTTTTGC
GAAGAATTTCCACGGTAAGAATCCAATCTCTCGAATTTAGGGTGA
AAGAAGCTTGGCATAGGGGTGTGCACGAACTCGGTGGAGGAAATT
TCCGCGGGGCAAGGCTTCGCGAAGCGGAGTCGCGGCAGTGGCTTT
GAAGATCTTTGGGAGCAGTCCTTGTGCGCTTACGAGGTGAGCCGG
TGGGGAACCGTTATCTGCCTATGGTGTGAGCCCCCCTAGAGAGCT
TCAAGAGCAATCAGCCCGACCTAGAAAGGAGGCCAAGAGAGAGAC
CCCTACGGGGGGAACCGTTTTCTGCCTACGAGATGGCACATTTAC
TGGGAAGCTTTACGGCGTCCTCGTGGAAGTTCAATGCCCGCAGAC
TTAAGTGCTCTATTCACGGTCTGACGTGACACGCTAAATTCAGAC
ATAGCTTCATTGATTGTCGGCCACGAGCCAGTCTCTCCCTCAACA
GTCATAAACCAACCTGCAATGGTCAAGCGATTTCCTTTAGCTTTC
CTAGCTTGTCGTTGACTGGACTTAGCTAGTTTTTCTCGCTGTGCT
CGGGCGTACTCACTGTTTGGGTCTTTCCAGCGTTCTGCGGCCTTT
TTACCGCCACGTCTTCCCATAGTGGCCAGAGCTTTTCGCCCTCGG
CTGCTCTGCGTCTCTGTCTGACGAGCAGGGACGACTGGCTGGCCT
TTAGCGACGTAGCCGCGCACACGTCGCGCCATCGTCTGGCGGTCA
CGCATCGGCGGCAGATCAGGCTCACGGCCGTCTGCTCCGACCGCC
TGAGCGACGGTGTAGGCACGCTCGTAGGCGTCGATGATCTTGGTG
TCTTTTAGGCGCTCACCAGCCGCTTTTAACTGGTATCCCACAGTC
AAAGCGTGGCGAAAAGCCGTCTCATCACGGGCGGCACGCCCTGGA
GCAGTCCAGAGGACACGGACGCCGTCGATCAGCTCTCCAGACGCT
TCAGCGGCGCTCGGCAGGCTTGCTTCAAGCGTGGCAAGTGCTTTT
GCTTCCGCAGTGGCTTTTCTTGCCGCTTCGATACGTGCCCGTCCG
CTAGAAAACTCCTGCTCATAGCGTTTTTTAGGTTTTTCTGTGCCT
GAGATCATGCGAGCAACCTCCATAAGATCAGCTAGGCGATCCACG
CGATTGTGCTGGGCATGCCAGCGGTACGCGGTGGGATCGTCGGAG
ACGTGCAGTGGCCACCGGCTCAGCCTATGTGAAAAAGCCTGGTCA
GCGCCGAAAACGCGGGTCATTTCCTCGGTCGTTGCAGCCAGCAGG
CGCATATTCGGGCTGCTCATGCCTGCTGCGGCATACACCGGATCA
ATGAGCCAGATGAGCTGGCATTTCCCGCTCAGTGGATTCACGCCG
ATCCAAGCTGGCGCTTTTTCCAGGCGTGCCCAGCGCTCCAAAATC
GCGTAGACCTCGGGGTTTACGTGCTCGATTTTCCCGCCGGCCTGG
TGGCTCGGCACATCAATGTCCAGGACAAGCACGGCTGCGTGCTGC
GCGTGCGTCAGAGCAACATACTGGCACCGGGCAAGCGATTTTGAA
CCAACTCGGTATAACTTCGGCTGTGTTTCTCCCGTGTCCGGGTCT
TTGATCCAAGCGCTGGCGAAGTCGCGGGTCTTGCTGCCCTGGAAA
TTTTCTCTGCCCAGGTGAGCGAGGAATTCGCGGCGGTCTTCGCTC
GTCCAGCCACGTGATCGCAGCGCGAGCTCGGGATGGGTGTCGAAC
AGATCAGCGGAAAATTTCCAGGCCGGTGTGTCAATGTCTCGTGAA
TCCGCTAGAGTCATTTTTGAGCGCTTTCTCCCAGGTTTGGACTGG
GGGTTAGCCGACGCCCTGTGAGTTACCGCTCACGGGGCGTTCAAC
ATTTTTCAGGTATTCGTGCAGCTTATCGCTTCTTGCCGCCTGTGC
GCTTTTTCGACGCGCGACGCTGCTGCCGATTCGGTGCAGGTGGTG
GCGGCGCTGACACGTCCTGGGCGGCCACGGCCACACGAAACGCGG
CATTTACGATGTTTGTCATGCCTGCGGGCACCGCGCCACGATCGC
GGATAATTCTCGCTGCCGCTTCCAGCTCTGTGACGACCATGGCCA
AAATTTCGCTCGGGGGACGCACTTCCAGCGCCATTTGCGACCTAG
CCGCCTCCAGCTCCTCGGCGTGGCGTTTGTTGGCGCGCTCGCGGC
TGGCTGCGGCACGACACGCATCTGAGCAATATTTTGCGCGCCGTC
CTCGCGGGTCAGGCCGGGGAGGAATCAGGCCACCGCAGTAGGCGC
AACTGATTCGATCCTCCACTACTGTGCGTCCTCCTGGCGCTGCCG
AGCACGCAGCTCGTCAGCCAGCTCCTCAAGATCCGCCACGAGAGT
TTCTAGGTCGCTCGCGGCACTGGCCCAGTCTCGTGATGCTGGCGC
GTCCGTCGTATCGAGAGCTCGGAAAAATCCGATCACCGTTTTTAA
ATCGACGGCAGCATCGAGCGCGTCGGACTCCAGCGCGACATCAGA
GAGATCCATAGCTGATGATTCGGGCCAATTTTGGTACTTCGTCGT
GAAGGTCATGACACCATTATAACGAACGTTCGTTAAAGTTTTTGG
CGGAAAATCACGCGGCACGAAAATTTTCACGAAGCGGGACTTTGC
GCAGCTCAGGGGTGCTAAAAATTTTGTATCGCACTTGATTTTTCC
GAAAGACAGATTATCTGCAAACGGTGTGTCGTATTTCTGGCTTGG
TTTTTAAAAAATCTGGAATCGAAAATTTGCGGGGCGA
pJC1-PT7-luc2-broccoli-TT7:
SEQ ID NO: 121
GGTACGCAACAAAAAAGCGTCAGGATCGCCGTAGAGCGATTGAAG
ACCGTCAACCAAAGGGGAAGCCTCCAATCGACGCGACGCGCGCTC
TACGGCGATCCTGACGCAGATTTTTAGCTATCTGTCGCAGCGCCC
TCAGGGACAAGCCACCCGCACAACGTCGCGAGGGCGATCAGCGAC
GCCGCAGGGGGATCCAGTACTCAGCTGGCTAACTAGAGAACCCAC
TGCTTACTGGCTTATCGAAATTAATACGACTCACTATAGGGAGAC
CCAAGCTGGCTAGCGTTTAAACTTAAGCTTGGCAATCCGGTACTG
TTGGTAAAGCCACCATGGAAGATGCCAAAAACATTAAGAAGGGCC
CAGCGCCATTCTACCCACTCGAAGACGGGACCGCCGGCGAGCAGC
TGCACAAAGCCATGAAGCGCTACGCCCTGGTGCCCGGCACCATCG
CCTTTACCGACGCACATATCGAGGTGGACATTACCTACGCCGAGT
ACTTCGAGATGAGCGTTCGGCTGGCAGAAGCTATGAAGCGCTATG
GGCTGAATACAAACCATCGGATCGTGGTGTGCAGCGAGAATAGCT
TGCAGTTCTTCATGCCCGTGTTGGGTGCCCTGTTCATCGGTGTGG
CTGTGGCCCCAGCTAACGACATCTACAACGAGCGCGAGCTGCTGA
ACAGCATGGGCATCAGCCAGCCCACCGTCGTATTCGTGAGCAAGA
AAGGGCTGCAAAAGATCCTCAACGTGCAAAAGAAGCTACCGATCA
TACAAAAGATCATCATCATGGATAGCAAGACCGACTACCAGGGCT
TCCAAAGCATGTACACCTTCGTGACTTCCCATTTGCCACCCGGCT
TCAACGAGTACGACTTCGTGCCCGAGAGCTTCGACCGGGACAAAA
CCATCGCCCTGATCATGAACAGTAGTGGCAGTACCGGATTGCCCA
AGGGCGTAGCCCTACCGCACCGCACCGCTTGTGTCCGATTCAGTC
ATGCCCGCGACCCCATCTTCGGCAACCAGATCATCCCCGACACCG
CTATCCTCAGCGTGGTGCCATTTCACCACGGCTTCGGCATGTTCA
CCACGCTGGGCTACTTGATCTGCGGCTTTCGGGTCGTGCTCATGT
ACCGCTTCGAGGAGGAGCTATTCTTGCGCAGCTTGCAAGACTATA
AGATTCAATCTGCCCTGCTGGTGCCCACACTATTTAGCTTCTTCG
CTAAGAGCACTCTCATCGACAAGTACGACCTAAGCAACTTGCACG
AGATCGCCAGCGGCGGGGCGCCGCTCAGCAAGGAGGTAGGTGAGG
CCGTGGCCAAACGCTTCCACCTACCAGGCATCCGCCAGGGCTACG
GCCTGACAGAAACAACCAGCGCCATTCTGATCACCCCCGAAGGGG
ACGACAAGCCTGGCGCAGTAGGCAAGGTGGTGCCCTTCTTCGAGG
CTAAGGTGGTGGACTTGGACACCGGTAAGACACTGGGTGTGAACC
AGCGCGGCGAGCTGTGCGTCCGTGGCCCCATGATCATGAGCGGCT
ACGTTAACAACCCCGAGGCTACAAACGCTCTCATCGACAAGGACG
GCTGGCTGCACAGCGGCGACATCGCCTACTGGGACGAGGACGAGC
ACTTCTTCATCGTGGACCGGCTGAAGAGCCTGATCAAATACAAGG
GCTACCAGGTAGCCCCAGCCGAACTGGAGAGCATCCTGCTGCAAC
ACCCCAACATCTTCGACGCCGGGGTCGCCGGCCTGCCCGACGACG
ATGCCGGCGAGCTGCCCGCCGCAGTCGTCGTGCTGGAACACGGTA
AAACCATGACCGAGAAGGAGATCGTGGACTATGTGGCCAGCCAGG
TTACAACCGCCAAGAAGCTGCGCGGTGGTGTTGTGTTCGTGGACG
AGGTGCCTAAAGGACTGACCGGCAAGTTGGACGCCCGCAAGATCC
GCGAGATTCTCATTAAGGCCAAGAAGGGCGGCAAGATCGCCGTGT
AATAATTCTAGATACGTATTGCCATGTGTATGTGGGAGACGGTCG
GGTCCAGATATTCGTATCTGTCGAGTAGAGTGTGGGCTCCCACAT
ACTCTGATGATCCTTCGGGATCATTCATGGCAAGTCGACCTAGCA
TAACCCCTTGGGGCCTCTAAACGGGTCTTGAGGGGTTTTTTGCTG
AAAGATATCGATATCCAATGGCAACAACGTTGCGCAAACTATTAA
CTGGCGAACTACTTACTCTAGCTTCCCGGCAACAATTAATAGACT
GGATGGAGGCGGATAAAGTTGCAGGACCACTTCTGCGCTCGGCCC
TTCCGGCTGGCTGGTTTATTGCTGATAAATCTGGAGCCGGTGAGC
GTGGGTCTCGCGGTATCATTGCAGCACTGGGGCCAGATGGTAAGC
CCTCCCGTATCGTAGTTATCTACACGACGGGGAGTCAGGCAACTA
TGGATGAACGAAATAGACAGATCGCTGAGATAGGTGCCTCACTGA
TTAAGCATTGGTAACTGTCAGACCAAGTTTACTCATATATACTTT
AGATTGATTTAAAACTTCATTTTTAATTTAAAAGGATCTAGGTGA
AGATCCTTTTTGATAATCTCATGACCAAAATCCCTTAACGTGAGT
TTTCGTTCCACTGAGCGTCAGACCCCTTAATAAGATGATCTTCTT
GAGATCGTTTTGGTCTGCGCGTAATCTCTTGCTCTGAAAACGAAA
AAACCGCCTTGCAGGGCGGTTTTTCGAAGGTTCTCTGAGCTACCA
ACTCTTTGAACCGAGGTAACTGGCTTGGAGGAGCGCAGTCACCAA
AACTTGTCCTTTCAGTTTAGCCTTAACCGGCGCATGACTTCAAGA
CTAACTCCTCTAAATCAATTACCAGTGGCTGCTGCCAGTGGTGCT
TTTGCATGTCTTTCCGGGTTGGACTCAAGACGATAGTTACCGGAT
AAGGCGCAGCGGTCGGACTGAACGGGGGGTTCGTGCATACAGTCC
AGCTTGGAGCGAACTGCCTACCCGGAACTGAGTGTCAGGCGTGGA
ATGAGACAAACGCGGCCATAACAGCGGAATGACACCGGTAAACCG
AAAGGCAGGAACAGGAGAGCGCACGAGGGAGCCGCCAGGGGGAAA
CGCCTGGTATCTTTATAGTCCTGTCGGGTTTCGCCACCACTGATT
TGAGCGTCAGATTTCGTGATGCTTGTCAGGGGGGCGGAGCCTATG
GAAAAACGGCTTTGCCGCGGCCCTCTCACTTCCCTGTTAAGTATC
TTCCTGGCATCTTCCAGGAAATCTCCGCCCCGTTCGTAAGCCATT
TCCGCTCGCCGCAGTCGAACGACCGAGCGTAGCGAGTCAGTGAGC
GAGGAAGCGGAATATATCCTGTATCACATATTCTGCTGACGCACC
GGTGCAGCCTTTTTTCTCCTGCCACATGAAGCACTTCACTGACAC
CCTCATCAGTGCCAACATAGTAAGCCAGTATACACTCCGCTAGCG
CTGAGGTCTGCCTCGTGAAGAAGGTGTTGCTGACTCATACCAGGC
CTGAATCGCCCCATCATCCAGCCAGAAAGTGAGGGAGCCACGGTT
GATGAGAGCTTTGTTGTAGGTGGACCAGTTGGTGATTTTGAACTT
TTGCTTTGCCACGGAACGGTCTGCGTTGTCGGGAAGATGCGTGAT
CTGATCCTTCAACTCAGCAAAAGTTCGATTTATTCAACAAAGCCA
CGTTGTGTCTCAAAATCTCTGATGTTACATTGCACAAGATAAAAA
TATATCATCATGAACAATAAAACTGTCTGCTTACATAAACAGTAA
TACAAGGGGTGTTATGAGCCATATTCAACGGGAAACGTCTTGCTC
GAGGCCGCGATTAAATTCCAACATGGATGCTGATTTATATGGGTA
TAAATGGGCTCGCGATAATGTCGGGCAATCAGGTGCGACAATCTA
TCGATTGTATGGGAAGCCCGATGCGCCAGAGTTGTTTCTGAAACA
TGGCAAAGGTAGCGTTGCCAATGATGTTACAGATGAGATGGTCAG
ACTAAACTGGCTGACGGAATTTATGCCTCTTCCGACCATCAAGCA
TTTTATCCGTACTCCTGATGATGCATGGTTACTCACCACTGCGAT
CCCCGGGAAAACAGCATTCCAGGTATTAGAAGAATATCCTGATTC
AGGTGAAAATATTGTTGATGCGCTGGCAGTGTTCCTGCGCCGGTT
GCATTCGATTCCTGTTTGTAATTGTCCTTTTAACAGCGATCGCGT
ATTTCGTCTCGCTCAGGCGCAATCACGAATGAATAACGGTTTGGT
TGATGCGAGTGATTTTGATGACGAGCGTAATGGCTGGCCTGTTGA
ACAAGTCTGGAAAGAAATGCATAAGCTTTTGCCATTCTCACCGGA
TTCAGTCGTCACTCATGGTGATTTCTCACTTGATAACCTTATTTT
TGACGAGGGGAAATTAATAGGTTGTATTGATGTTGGACGAGTCGG
AATCGCAGACCGATACCAGGATCTTGCCATCCTATGGAACTGCCT
CGGTGAGTTTTCTCCTTCATTACAGAAACGGCTTTTTCAAAAATA
TGGTATTGATAATCCTGATATGAATAAATTGCAGTTTCATTTGAT
GCTCGATGAGTTTTTCTAATCAGAATTGGTTAATTGGTTGTAACA
CTGGCAGAGCATTACGCTGACTTGACGGGACGGCGGCTTTGTTGA
ATAAATCGAACTTTTGCTGAGTTGAAGGATCAGATCACGCATCTT
CCCGACAACGCAGACCGTTCCGTGGCAAAGCAAAAGTTCAAAATC
ACCAACTGGTCCACCTACAACAAAGCTCTCATCAACCGTGGCTCC
CTCACTTTCTGGCTGGATGATGGGGCGATTCAGGCCTGGTATGAG
TCAGCAACACCTTCTTCACGAGGCAGACCTCAGCGCTCAAAGATG
CAGGGGTAAAAGCTAACCGCATCTTTACCGACAAGGCATCCGGCA
GTTCAACAGATCGGGAAGGGCTGGATTTGCTGAGGATGAAGGTGG
AGGAAGGTGATGTCATTCTGGTGAAGAAGCTCGACCGTCTTGGCC
GCGACACCGCCGACATGATCCAACTGATAAAAGAGTTTGATGCTC
AGGGTGTAGCGGTTCGGTTTATTGACGACGGGATCAGTACCGACG
GTGATATGGGGCAAATGGTGGTCACCATCCTGTCGGCTGTGGCAC
AGGCTGAACGCCGGAGGATCAAGTCGGTCAAGCCAAGCGCAACCA
GCGGCACCGCCGCGAGCAACGTCGCAAGGGCGATCAGGGGACGAT
TTTTGCGAAGAATTTCCACGGTAAGAATCCAATCTCTCGAATTTA
GGGTGAAAGAAGCTTGGCATAGGGGTGTGCACGAACTCGGTGGAG
GAAATTTCCGCGGGGCAAGGCTTCGCGAAGCGGAGTCGCGGCAGT
GGCTTTGAAGATCTTTGGGAGCAGTCCTTGTGCGCTTACGAGGTG
AGCCGGTGGGGAACCGTTATCTGCCTATGGTGTGAGCCCCCCTAG
AGAGCTTCAAGAGCAATCAGCCCGACCTAGAAAGGAGGCCAAGAG
AGAGACCCCTACGGGGGGAACCGTTTTCTGCCTACGAGATGGCAC
ATTTACTGGGAAGCTTTACGGCGTCCTCGTGGAAGTTCAATGCCC
GCAGACTTAAGTGCTCTATTCACGGTCTGACGTGACACGCTAAAT
TCAGACATAGCTTCATTGATTGTCGGCCACGAGCCAGTCTCTCCC
TCAACAGTCATAAACCAACCTGCAATGGTCAAGCGATTTCCTTTA
GCTTTCCTAGCTTGTCGTTGACTGGACTTAGCTAGTTTTTCTCGC
TGTGCTCGGGCGTACTCACTGTTTGGGTCTTTCCAGCGTTCTGCG
GCCTTTTTACCGCCACGTCTTCCCATAGTGGCCAGAGCTTTTCGC
CCTCGGCTGCTCTGCGTCTCTGTCTGACGAGCAGGGACGACTGGC
TGGCCTTTAGCGACGTAGCCGCGCACACGTCGCGCCATCGTCTGG
CGGTCACGCATCGGCGGCAGATCAGGCTCACGGCCGTCTGCTCCG
ACCGCCTGAGCGACGGTGTAGGCACGCTCGTAGGCGTCGATGATC
TTGGTGTCTTTTAGGCGCTCACCAGCCGCTTTTAACTGGTATCCC
ACAGTCAAAGCGTGGCGAAAAGCCGTCTCATCACGGGCGGCACGC
CCTGGAGCAGTCCAGAGGACACGGACGCCGTCGATCAGCTCTCCA
GACGCTTCAGCGGCGCTCGGCAGGCTTGCTTCAAGCGTGGCAAGT
GCTTTTGCTTCCGCAGTGGCTTTTCTTGCCGCTTCGATACGTGCC
CGTCCGCTAGAAAACTCCTGCTCATAGCGTTTTTTAGGTTTTTCT
GTGCCTGAGATCATGCGAGCAACCTCCATAAGATCAGCTAGGCGA
TCCACGCGATTGTGCTGGGCATGCCAGCGGTACGCGGTGGGATCG
TCGGAGACGTGCAGTGGCCACCGGCTCAGCCTATGTGAAAAAGCC
TGGTCAGCGCCGAAAACGCGGGTCATTTCCTCGGTCGTTGCAGCC
AGCAGGCGCATATTCGGGCTGCTCATGCCTGCTGCGGCATACACC
GGATCAATGAGCCAGATGAGCTGGCATTTCCCGCTCAGTGGATTC
ACGCCGATCCAAGCTGGCGCTTTTTCCAGGCGTGCCCAGCGCTCC
AAAATCGCGTAGACCTCGGGGTTTACGTGCTCGATTTTCCCGCCG
GCCTGGTGGCTCGGCACATCAATGTCCAGGACAAGCACGGCTGCG
TGCTGCGCGTGCGTCAGAGCAACATACTGGCACCGGGCAAGCGAT
TTTGAACCAACTCGGTATAACTTCGGCTGTGTTTCTCCCGTGTCC
GGGTCTTTGATCCAAGCGCTGGCGAAGTCGCGGGTCTTGCTGCCC
TGGAAATTTTCTCTGCCCAGGTGAGCGAGGAATTCGCGGCGGTCT
TCGCTCGTCCAGCCACGTGATCGCAGCGCGAGCTCGGGATGGGTG
TCGAACAGATCAGCGGAAAATTTCCAGGCCGGTGTGTCAATGTCT
CGTGAATCCGCTAGAGTCATTTTTGAGCGCTTTCTCCCAGGTTTG
GACTGGGGGTTAGCCGACGCCCTGTGAGTTACCGCTCACGGGGCG
TTCAACATTTTTCAGGTATTCGTGCAGCTTATCGCTTCTTGCCGC
CTGTGCGCTTTTTCGACGCGCGACGCTGCTGCCGATTCGGTGCAG
GTGGTGGCGGCGCTGACACGTCCTGGGCGGCCACGGCCACACGAA
ACGCGGCATTTACGATGTTTGTCATGCCTGCGGGCACCGCGCCAC
GATCGCGGATAATTCTCGCTGCCGCTTCCAGCTCTGTGACGACCA
TGGCCAAAATTTCGCTCGGGGGACGCACTTCCAGCGCCATTTGCG
ACCTAGCCGCCTCCAGCTCCTCGGCGTGGCGTTTGTTGGCGCGCT
CGCGGCTGGCTGCGGCACGACACGCATCTGAGCAATATTTTGCGC
GCCGTCCTCGCGGGTCAGGCCGGGGAGGAATCAGGCCACCGCAGT
AGGCGCAACTGATTCGATCCTCCACTACTGTGCGTCCTCCTGGCG
CTGCCGAGCACGCAGCTCGTCAGCCAGCTCCTCAAGATCCGCCAC
GAGAGTTTCTAGGTCGCTCGCGGCACTGGCCCAGTCTCGTGATGC
TGGCGCGTCCGTCGTATCGAGAGCTCGGAAAAATCCGATCACCGT
TTTTAAATCGACGGCAGCATCGAGCGCGTCGGACTCCAGCGCGAC
ATCAGAGAGATCCATAGCTGATGATTCGGGCCAATTTTGGTACTT
CGTCGTGAAGGTCATGACACCATTATAACGAACGTTCGTTAAAGT
TTTTGGCGGAAAATCACGCGGCACGAAAATTTTCACGAAGCGGGA
CTTTGCGCAGCTCAGGGGTGCTAAAAATTTTGTATCGCACTTGAT
TTTTCCGAAAGACAGATTATCTGCAAACGGTGTGTCGTATTTCTG
GCTTGGTTTTTAAAAAATCTGGAATCGAAAATTTGCGGGGCGACC
GAGAAGTTTTTTACAAAAGGCAAAAACTTTTTCGGGATCGACAGA
AATAAAACGATCGAC
5′UTR-egfp (modified from
Aequorea victoria):
SEQ ID NO: 112
GGAGACCCAAGCTGGCTAGCGTTTAAACTTAAGCTTCGAATTCTG
CAGTCGACGGTACCGCGGGCCCGGGATCCACCGGTCGCCACCATG
GTGAGCAAGGGCGAGGAGCTGTTCACCGGGGTGGTGCCCATCCTG
GTCGAGCTGGACGGCGACGTAAACGGCCACAAGTTCAGCGTGTCC
GGCGAGGGCGAGGGCGATGCCACCTACGGCAAGCTGACCCTGAAG
TTCATCTGCACCACCGGCAAGCTGCCCGTGCCCTGGCCCACCCTC
GTGACCACCCTGACCTACGGCGTGCAGTGCTTCAGCCGCTACCCC
GACCACATGAAGCAGCACGACTTCTTCAAGTCCGCCATGCCCGAA
GGCTACGTCCAGGAGCGCACCATCTTCTTCAAGGACGACGGCAAC
TACAAGACCCGCGCCGAGGTGAAGTTCGAGGGCGACACCCTGGTG
AACCGCATCGAGCTGAAGGGCATCGACTTCAAGGAGGACGGCAAC
ATCCTGGGGCACAAGCTGGAGTACAACTACAACAGCCACAACGTC
TATATCATGGCCGACAAGCAGAAGAACGGCATCAAGGTGAACTTC
AAGATCCGCCACAACATCGAGGACGGCAGCGTGCAGCTCGCCGAC
CACTACCAGCAGAACACCCCCATCGGCGACGGCCCCGTGCTGCTG
CCCGACAACCACTACCTGAGCACCCAGTCCGCCCTGAGCAAAGAC
CCCAACGAGAAGCGCGATCACATGGTCCTGCTGGAGTTCGTGACC
GCCGCCGGGATCACTCTCGGCATGGACGAGCTGTACAAGTAA
5′UTR-luc2 (Photinus pyralis):
SEQ ID NO: 82
GGAGACCCAAGCTGGCTAGCGTTTAAACTTAAGCTTGGCAATCCG
GTACTGTTGGTAAAGCCACCATGGAAGATGCCAAAAACATTAAGA
AGGGCCCAGCGCCATTCTACCCACTCGAAGACGGGACCGCCGGCG
AGCAGCTGCACAAAGCCATGAAGCGCTACGCCCTGGTGCCCGGCA
CCATCGCCTTTACCGACGCACATATCGAGGTGGACATTACCTACG
CCGAGTACTTCGAGATGAGCGTTCGGCTGGCAGAAGCTATGAAGC
GCTATGGGCTGAATACAAACCATCGGATCGTGGTGTGCAGCGAGA
ATAGCTTGCAGTTCTTCATGCCCGTGTTGGGTGCCCTGTTCATCG
GTGTGGCTGTGGCCCCAGCTAACGACATCTACAACGAGCGCGAGC
TGCTGAACAGCATGGGCATCAGCCAGCCCACCGTCGTATTCGTGA
GCAAGAAAGGGCTGCAAAAGATCCTCAACGTGCAAAAGAAGCTAC
CGATCATACAAAAGATCATCATCATGGATAGCAAGACCGACTACC
AGGGCTTCCAAAGCATGTACACCTTCGTGACTTCCCATTTGCCAC
CCGGCTTCAACGAGTACGACTTCGTGCCCGAGAGCTTCGACCGGG
ACAAAACCATCGCCCTGATCATGAACAGTAGTGGCAGTACCGGAT
TGCCCAAGGGCGTAGCCCTACCGCACCGCACCGCTTGTGTCCGAT
TCAGTCATGCCCGCGACCCCATCTTCGGCAACCAGATCATCCCCG
ACACCGCTATCCTCAGCGTGGTGCCATTTCACCACGGCTTCGGCA
TGTTCACCACGCTGGGCTACTTGATCTGCGGCTTTCGGGTCGTGC
TCATGTACCGCTTCGAGGAGGAGCTATTCTTGCGCAGCTTGCAAG
ACTATAAGATTCAATCTGCCCTGCTGGTGCCCACACTATTTAGCT
TCTTCGCTAAGAGCACTCTCATCGACAAGTACGACCTAAGCAACT
TGCACGAGATCGCCAGCGGCGGGGCGCCGCTCAGCAAGGAGGTAG
GTGAGGCCGTGGCCAAACGCTTCCACCTACCAGGCATCCGCCAGG
GCTACGGCCTGACAGAAACAACCAGCGCCATTCTGATCACCCCCG
AAGGGGACGACAAGCCTGGCGCAGTAGGCAAGGTGGTGCCCTTCT
TCGAGGCTAAGGTGGTGGACTTGGACACCGGTAAGACACTGGGTG
TGAACCAGCGCGGCGAGCTGTGCGTCCGTGGCCCCATGATCATGA
GCGGCTACGTTAACAACCCCGAGGCTACAAACGCTCTCATCGACA
AGGACGGCTGGCTGCACAGCGGCGACATCGCCTACTGGGACGAGG
ACGAGCACTTCTTCATCGTGGACCGGCTGAAGAGCCTGATCAAAT
ACAAGGGCTACCAGGTAGCCCCAGCCGAACTGGAGAGCATCCTGC
TGCAACACCCCAACATCTTCGACGCCGGGGTCGCCGGCCTGCCCG
ACGACGATGCCGGCGAGCTGCCCGCCGCAGTCGTCGTGCTGGAAC
ACGGTAAAACCATGACCGAGAAGGAGATCGTGGACTATGTGGCCA
GCCAGGTTACAACCGCCAAGAAGCTGCGCGGTGGTGTTGTGTTCG
TGGACGAGGTGCCTAAAGGACTGACCGGCAAGTTGGACGCCCGCA
AGATCCGCGAGATTCTCATTAAGGCCAAGAAGGGCGGCAAGATCG
CCGTGTAA

b) Transformation of Plasmids pJC1-PT7-egfp-broccoli-TT7 and pJC1-PT7-Luc2-broccoli-TT7 in Corynebacterium glutamicum ATCC 13032(DE3)_Δcg2273

The construction of C. glutamicum ATCC 13032(DE3)_Δcg2273 was described in example 5b. Competent cells of the C. glutamicum strain ATCC 13032(DE3)_Δcg2273 were prepared and transformed by electroporation with vectors pJC1-PT7-egfp-broccoli-TT7 or with pJC1-PT7-luc2-broccoli-TT7 according to Tauch et al., 2002 (Tauch, A., Kirchner, O., Löffler, B., Götker, S., Pühler, A., and Kalinowski, J. Efficient electrotransformation of Corynebacterium diphtheriae with a Mini-Replicon Derived from the Corynebacterium glutamicum plasmid pGA1. Curr. Microbiol. 2002; 45, 362-367). The selection of the transformants was carried out on BHI agar (1%) plates with 25 μg/ml of kanamycin. Clones thus obtained were named C. glutamicum ATCC 13032(DE3)_Δcg2273_pJC1_PT7-egfp-broccoli-TT7 and C. glutamicum ATCC 13032(DE3)_Δcg2273_pJC1_PT7-luc2-broccoli-TT7.

### c) Cultivation and Phenotypic Validation of Cells Using Fluorescent Activated Cell Sorting (FACS)

The produced strains C. glutamicum ATCC 13032(DE3)_Δcg2273_pJC1_PT7-egfp-broccoli-TT7 and C. glutamicum ATCC 13032(DE3)_Δcg2273_pJC1_PT7-luc2-broccoli-TT7 were streaked on BHI agar plates containing 25 μg/ml kanamycin, which were cultivated at 30° C. overnight. Grown cells were resuspended in CGIII cultivation medium containing 25 μg/ml kanamycin and the OD₆₀₀ was adjusted to 0.75 in a tube containing 2 mL CGIII cultivation medium with antibiotic. Cells were incubated at 30° C. and 120 rpm and induced by 1.5 mM IPTG after six hours of cultivation. Afterwards, incubation was continued for six hours. Subsequently, cells were diluted to an OD₆₀₀ of 0.6 using PBS with a final concentration of 500 μM DFHBI. Cells were analyzed using an AriaIII High-speed cell sorter as already described in example 1d). DFHBI-stained cells showed a significantly increased fluorescent output compared to unstained cells (cf. FIG. 7).

### d) Extraction of RNA and Analysis by RT-PCR

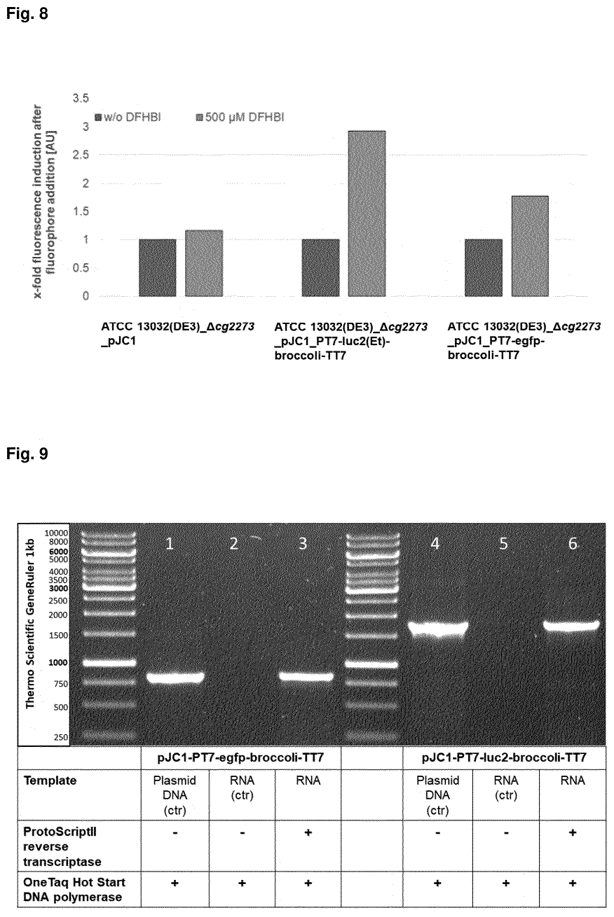

RNA was isolated from 1.38×10⁹ cells according to example 1f). For verification of the resulting RNA fragments, reverse transcriptase PCR (RT-PCR) was performed using the One Taq One-Step RT-PCR kit (New England Biolabs, Ipswich, MA, USA) with primers egfp_for (SEQ ID NO: 109) and broccoli_rev (SEQ ID NO: 113) to verify an internal part the egfp-broccoli fragment and primers luc2_for (SEQ ID NO: 114) and broccoli_rev (SEQ ID NO: 113) to verify an internal part of the luc2-broccoli fragment. Initially, cDNA was produced from RNA using ProtoScriptII reverse transcriptase, and subsequently, resulting cDNA fragments were amplified by OneTaq Hot Start DNA polymerase using primers mentioned above. The amplified fragments with a size of 831 bp for C. glutamicum ATCC 13032(DE3)_Δcg2273_pJC1_PT7-egfp-broccoli-TT7 (total transcript length of 973 nts) and 1774 bp for C. glutamicum ATCC 13032(DE3)_Δcg2273_pJC1_PT7-luc2-broccoli-TT7 (total transcript length of 1894 nts) was identified in the prepared RNA samples (cf. FIG. 8). As positive control, vectors pJC1_PT7-egfp-broccoli-TT7 and pJC1_PT7-luc2-broccoli-TT7 were amplified by OneTaq Hot Start DNA polymerase using primers mentioned above. As negative control, RNA samples were amplified by OneTaq Hot Start DNA polymerase without performing the reverse transcriptase step. No bands appeared on agarose gel demonstrating that prepared RNA samples were free from original vector DNA (cf. FIG. 8).

egfp_for:
SEQ ID NO: 109
ATGGTGAGCAAGGGCGAGGA
broccoli_rev:
SEQ ID NO: 113
TTGCCATGAATGATCCCGAAG
luc2_for:
SEQ ID NO: 114
ATGGAAGATGCCAAAAACATTAAGAAG

This experiment shows the successful linking of a hitherto unsuspicious phenotype (RNA production) with a fluorescence output. In this experiment, the produced RNA has a length of 973 or 1894 nucleotides and is transcribed from a vector. In accordance with the procedure shown in example 1, the optimization of the production of RNA with a length of at least 1894 nucleotides is therefore possible using the invention.

1. A method for optimizing the production of a heterologous RNA sequence of interest in a cell, comprising the steps of:
a) introducing into a plurality of cells a vector capable of expressing a heterologous RNA of interest, wherein said RNA is tagged with a RNA tag comprising
an aptamer capable of stabilizing a fluorophore and
a scaffold capable of stabilizing the aptamer;
b) culturing the cells in a culture medium under conditions that allow expression of the heterologous RNA of interest;
c) adding said fluorophore to the culture medium;
d) identifying those cells that show the highest intensity of fluorescence and therefore a high expression of the RNA of interest.

2. A method for producing a heterologous RNA of interest, comprising the steps of
a) introducing into a plurality of cells a vector capable of expressing a heterologous RNA of interest, wherein said RNA is tagged with a RNA tag comprising an aptamer capable of stabilizing a fluorophore and a scaffold capable of stabilizing the aptamer;
b) culturing the cells in a culture medium under conditions that allow expression of the heterologous RNA of interest;
c) adding said fluorophore to the culture medium;
d) identifying and isolating those cells that show the highest intensity of fluorescence and therefore a high expression of the RNA of interest;
e) removing the first vector of step a) from the cells isolated in step d);
f) introducing a second vector capable of expressing the heterologous RNA of interest without the RNA tag into the cells obtained in step e);
g) producing the RNA of interest by culturing the cells obtained in step f).

3. A method for comparing the production capacity of different cells for a heterologous RNA sequence of interest, comprising the steps of:
a) introducing into a plurality of cells a vector capable of expressing a heterologous RNA of interest, wherein said RNA is tagged with a RNA tag comprising
an aptamer capable of stabilizing a fluorophore and
a scaffold capable of stabilizing the aptamer;
b) culturing the cells in a culture medium under conditions that allow expression of the RNA of interest;
c) adding said fluorophore to the culture medium;
d) comparing the intensity of fluorescence between the plurality of cells.

4. The method according to claim 1, wherein the cell is a microbial cell, in particular a gram positive bacterial cell.

5. The method according to claim 1, wherein in step d) the cells showing the highest intensity of fluorescence are identified using flow cytometry.

6. The method according to claim 1, wherein the cells show different levels of expression of the heterologous RNA due to culture conditions, in particular temperature, pressure and/or culture medium, chromosomal genetic alterations or genetic alterations in the vector.

7. The method according to claim 1, wherein the aptamer comprises a sequence according to SEQ ID NO: 1 to 8, 13 to 16, 65 or 66.

8. The method according to claim 1, wherein the RNA scaffold capable of stabilizing the aptamer comprises the sequence according to SEQ ID NO: 9 to 12.

9. The method according to claim 1, wherein the RNA tag comprises a sequence according to SEQ ID NO: 17 to 64, 69, 77 or 78.

10. A cell harboring a vector capable of expressing a heterologous RNA of interest, wherein said RNA is tagged with a RNA tag comprising
an aptamer capable of stabilizing a fluorophore and
a RNA scaffold capable of stabilizing the aptamer.

11. The cell according to claim 10, wherein the cell is a Gram positive bacterial cell from the genus Corynebacterium, in particular Corynebacterium glutamicum.

12. The cell according to claim 10, wherein the aptamer comprises a sequence according to SEQ ID NO: 1 to 8, 13 to 16, 65 or 66.

13. The cell according to claim 10, wherein the aptamer is capable of stabilizing any fluorophore selected from 2-HBI, DFHBI, DFHBI-1T, DFHBI-2T, DMABI or DMHBI, TO1, TO3 or Hoechst 1C.

14. The cell according to claim 10, wherein the RNA scaffold capable of stabilizing the aptamer comprises the sequence according to SEQ ID NO: 9 to 12.

15. The cell according to claim 10, wherein the RNA tag comprises a sequence according to SEQ ID NO: 17 to 64, 69, 77 or 78.
